(19) United States
(12) Patent Application Publication
(10) Pub. No.: US 2004/0214759 A1
(43) Pub. Date: Oct. 28, 2004

(54) Compositions and methods of use for a fibroblast growth factor

(76) Inventors: John P. Alsobrook II, Madison, CT (US); Enrique Alvarez, Clinton, CT (US); David W. Anderson, Branford, CT (US); Joel S. Bader, Stamford, CT (US); Ferenc L. Boldog, North Haven, CT (US); Catherine E. Burgess, Wethersfield, CT (US); Rajeev A. Chillakuru, Chester, CT (US); Lisa Deegler, Stamford, CT (US); Shlomit R. Edinger, New Haven, CT (US); Elma R. Fernandes, Branford, CT (US); Linda Gorman, Branford, CT (US); William M. Grosse, Branford, CT (US); John L. Herrmann, Guilford, CT (US); Michael E. Jeffers, Branford, CT (US); William J. LaRochelle, Madison, CT (US); Denise M. Lepley, Branford, CT (US); Henri S. Lichenstein, Guilford, CT (US); Kumar Namdev, Killingworth, CT (US); Muralidhara Padigaru, Branford, CT (US); Carol E. A. Pena, New Haven, CT (US); Sudhirdas K. Prayaga, O'Fallon, MO (US); Daniel K. Rieger, Branford, CT (US); Richard A. Shimkets, Guilford, CT (US); Pascal Valax, Biarritz (FR); Meijia Yang, East Lyme, CT (US); Zachary Yim, Guilford, CT (US); Mei Zhong, Branford, CT (US)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/435,087

(22) Filed: May 9, 2003

Related U.S. Application Data
(60) Provisional application No. 60/378,996, filed on May 9, 2002.
(60) Provisional application No. 60/379,356, filed on May 10, 2002.
(60) Provisional application No. 60/385,173, filed on May 31, 2002.
(60) Provisional application No. 60/390,597, filed on Jun. 21, 2002.
(60) Provisional application No. 60/421,008, filed on Oct. 24, 2002.
(60) Provisional application No. 60/425,811, filed on Nov. 13, 2002.

(51) Int. Cl. A61K 38/17

(52) U.S. Cl. 514/12

(57) ABSTRACT

In methods of using a fibroblast growth factor, such as for treating, preventing or delaying a proliferation-associated disorder, steps are provided to administer to a subject a therapeutically effective amount of a particular fibroblast growth factor polypeptide, or variant or fragment thereof.

### RELATED APPLICATIONS

This application claims the benefit of priority from U.S. Provisional applications U.S. Ser. No. 60/378,996, filed May 9, 2002 (Cura-57 A-UA); U.S. Ser. No. 60/379,356, filed May 10, 2002 (Cura-57 UDA); U.S. Ser. No. 60/385,173, filed May 31, 2002 (Cura-57 UE); U.S. Ser. No. 60/390,597, filed Jun. 21, 2002 (Cura-57 UDB); U.S. Ser. No. 60/421,008, filed Oct. 24, 2002 (Cura-57 UF); and U.S. Ser. No. 60/425,811, filed Nov. 13, 2002 (Cura-57 UH).

### FIELD OF THE INVENTION

The present invention generally relates to compositions and methods of treatment including but not restricted to oral mucositis, in mammals using growth factor-related polypeptides. More specifically, the nucleic acids and the polypeptides employed in the compositions and methods of the invention are related to a member of the fibroblast growth factor family.

### BACKGROUND OF THE INVENTION

The FGF family of proteins, whose prototypic members include acidic FGF (FGF-1) and basic FGF (FGF-2), bind to four related receptor tyrosine kinases. These FGF receptors are expressed on most types of cells in tissue culture. Dimerization of FGF receptor monomers upon ligand binding has been reported to be a requisite for activation of the kinase domains, leading to receptor trans phosphorylation. FGF receptor-1 (FGFR-1), which shows the broadest expression pattern of the four FGF receptors, contains at least seven tyrosine phosphorylation sites. A number of signal transduction molecules are affected by binding with different affinities to these phosphorylation sites.

Expression of FGFs and their receptors in brains of perinatal and adult mice has been examined. Messenger RNA all FGF genes, with the exception of FGF-4, is detected in these tissues. FGF-3, FGF-6, FGF-7 and FGF-8 genes demonstrate higher expression in the late embryonic stages than in postnatal stages, suggesting that these members are involved in the late stages of brain development. In contrast, expression of FGF-1 and FGF-5 increased after birth. In particular, FGF-6 expression in perinatal mice has been reported to be restricted to the central nervous system and skeletal muscles, with intense signals in the developing cerebrum in embryos but in cerebellum in 5-day-old neonates. FGF-receptor (FGFR)-4, a cognate receptor for FGF-6, demonstrate similar spatiotemporal expression, suggesting that FGF-6 and FGFR-4 plays significant roles in the maturation of nervous system as a ligand-receptor system. According to Ozawa et al., these results strongly suggest that the various FGFs and their receptors are involved in the regulation of a variety of developmental processes of brain, such as proliferation and migration of neuronal progenitor cells, neuronal and glial differentiation, neurite extensions, and synapse formation.

Other members of the FGF polypeptide family include the FGF receptor tyrosine kinase (FGFRTK) family and the FGF receptor heparan sulfate proteoglycan (FGFRHS) family. These members interact to regulate active and specific FGFR signal transduction complexes. These regulatory activities are diversified throughout a broad range of organs and tissues, and in both normal and tumor tissues, in mammals. Regulated alternative messenger RNA (mRNA) splicing and combination of variant subdomains give rise to diversity of FGFRTK monomers. Divalent cations cooperate with the FGFRHS to conformationally restrict FGFRTK trans-phosphorylation, which causes depression of kinase activity and facilitates appropriate activation of the FGFR complex by FGF. For example, it is known that different point mutations in the FGFRTK commonly cause craniofacial and skeletal abnormalities of graded severity by graded increases in FGF-independent activity of total FGFR complexes. Other processes in which FGF family exerts important effects are liver growth and function and prostate tumor progression.

Glia-activating factor (GAF), another FGF family member, is a heparin-binding growth factor that was purified from the culture supernatant of a human glioma cell line. See, Miyamoto et al., 1993, Mol Cell Biol 13(7): 4251-4259. GAF shows a spectrum of activity slightly different from those of other known growth factors, and is designated as FGF-9. The human FGF-9 cDNA encodes a polypeptide of 208 amino acids. Sequence similarity to other members of the FGF family was estimated to be around 30%. Two cysteine residues and other consensus sequences found in other family members were also well conserved in the FGF-9 sequence. FGF-9 was found to have no typical signal sequence in its N terminus like those in acidic FGF and basic FGF. Acidic FGF and basic FGF are known not to be secreted from cells in a conventional manner. However, FGF-9 was found to be secreted efficiently from cDNA-transfected COS cells despite its lack of a typical signal sequence. It could be detected exclusively in the culture medium of cells. The secreted protein lacked no amino acid residues at the N terminus with respect to those predicted by the cDNA sequence, except the initiation methionine. The rat FGF-9 cDNA was also cloned, and the structural analysis indicated that the FGF-9 gene is highly conserved.

### SUMMARY OF THE INVENTION

The present invention is based, in part, upon the discovery of a nucleic acid encoding a FGF-CX polypeptide having homology to Fibroblast Growth Factor (FGF) protein. Fibroblast Growth Factor-CX (FGF-CX) polynucleotide sequences and the FGF-CX polypeptides encoded by these nucleic acid sequences, and fragments, homologs, analogs, and derivatives thereof, are claimed in the invention.

In one aspect, the invention provides an isolated FGF-CX nucleic acid (SEQ ID NOs: 1, 3, 5, 7, 9, 11, 13, 15, 17, 19, 21, 23, 25, 27, 29, 31, 33, 35, as shown in Table A), that encodes a FGF-CX polypeptide, or a fragment, homolog, analog or derivative thereof. The nucleic acid can include, e.g., nucleic acid sequence encoding a polypeptide at least 85% identical to a polypeptide comprising the amino acid sequence of Table A (SEQ ID NOs: 2, 4, 6, 8, 10, 12, 14, 16, 18, 20, 22, 24, 26, 28, 30, 32, 34, 36). The nucleic acid can be, e.g., a genomic DNA fragment, or it can be a cDNA molecule.

Also included in the invention is a vector containing one or more of the nucleic acids described herein, and a cell containing the vectors or nucleic acids described herein.

The present invention is also directed to host cells transformed with a recombinant expression vector comprising any of the nucleic acid molecules described above.

In one aspect, the invention includes a pharmaceutical composition that includes a FGF-CX nucleic acid and a pharmaceutically acceptable carrier or diluent. In a further aspect, the invention includes a substantially purified FGF-CX polypeptide, e.g. any of the FGF-CX polypeptides encoded by a FGF-CX nucleic acid, and fragments, homologs, analogs, and derivatives thereof. The invention also includes a pharmaceutical composition that includes a FGF-CX polypeptide and a pharmaceutically acceptable carrier or diluent.

In a further aspect, the invention provides an antibody that binds specifically to a FGF-CX polypeptide. The antibody can be, e.g. a monoclonal or polyclonal antibody, and fragments, homologs, analogs, and derivatives thereof. The invention also includes a pharmaceutical composition including FGF-CX antibody and a pharmaceutically acceptable carrier or diluent. The present invention is also directed to isolated antibodies that bind to an epitope on a polypeptide encoded by any of the nucleic acid molecules described above.

The present invention is further directed to kits comprising antibodies that bind to a polypeptide encoded by any of the nucleic acid molecules described above and a negative control antibody.

The invention further provides a method for producing a FGF-CX polypeptide. The method includes providing a cell containing a FGF-CX nucleic acid, e.g., a vector that includes a FGF-CX nucleic acid, and culturing the cell under conditions sufficient to express the FGF-CX polypeptide encoded by the nucleic acid. The expressed FGF-CX polypeptide is then recovered from the cell. Preferably, the cell produces little or no endogenous FGF-CX polypeptide. The cell can be, e.g., a prokaryotic cell or eukaryotic cell.

The present invention provides a method of inducing an immune response in a mammal against a polypeptide encoded by any of the nucleic acid molecules disclosed above by administering to the mammal an amount of the polypeptide sufficient to induce the immune response.

The present invention is also directed to methods of identifying a compound that binds to FGF-CX polypeptide by contacting the FGF-CX polypeptide with a compound and determining whether the compound binds to the FGF-CX polypeptide.

The invention further provides methods of identifying a compound that modulates the activity of a FGF-CX polypeptide by contacting FGF-CX polypeptide with a compound and determining whether the FGF-CX polypeptide activity is modified.

The present invention is also directed to compounds that modulate FGF-CX polypeptide activity identified by contacting a FGF-CX polypeptide with the compound and determining whether the compound modifies activity of the FGF-CX polypeptide, binds to the FGF-CX polypeptide, or binds to a nucleic acid molecule encoding a FGF-CX polypeptide.

In another aspect, the invention provides a method of diagnosing a tissue proliferation-associated disorder, such as tumors, restenosis, psoriasis, diabetic and post-surgery complications, and rheumatoid arthritis, in a subject. The method includes providing a protein sample from the subject and measuring the amount of FGF-CX polypeptide in the subject sample. The amount of FGF-CX in the subject sample is then compared to the amount of FGF-CX polypeptide in a control protein sample. An alteration in the amount of FGF-CX polypeptide in the subject protein sample relative to the amount of FGF-CX polypeptide in the control protein sample indicates the subject has a tissue proliferation-associated condition. A control sample is preferably taken from a matched individual, i.e., an individual of similar age, sex, or other general condition but who is not suspected of having a tissue proliferation-associated condition. Alternatively, the control sample may be taken from the subject at a time when the subject is not suspected of having a tissue proliferation-associated disorder. In some embodiments, the FGF-CX polypeptide is detected using a FGF-CX antibody.

The invention is also directed to methods of inducing an immune response in a mammal against a polypeptide encoded by any of the nucleic acid molecules described above. The method includes administering to the mammal an amount of the polypeptide sufficient to induce the immune response.

In a further aspect, the invention includes a method of diagnosing a tissue proliferation-associated disorder, such as tumors, restenosis, psoriasis, diabetic and post-surgery complications, and rheumatoid arthritis, in a subject. The method includes providing a nucleic acid sample, e.g., RNA or DNA, or both, from the subject and measuring the amount of the FGF-CX nucleic acid in the subject nucleic acid sample. The amount of FGF-CX nucleic acid sample in the subject nucleic acid is then compared to the amount of FGF-CX nucleic acid in a control sample. An alteration in the amount of FGF-CX nucleic acid in the sample relative to the amount of FGF-CX in the control sample indicates the subject has a tissue proliferation-associated disorder.

In a further aspect, the invention includes a method of diagnosing a tissue proliferation-associated disorder in a subject. The method includes providing a nucleic acid sample from the subject and identifying at least a portion of the nucleotide sequence of a FGF-CX nucleic acid in the subject nucleic acid sample. The FGF-CX nucleotide sequence of the subject sample is then compared to a FGF-CX nucleotide sequence of a control sample. An alteration in the FGF-CX nucleotide sequence in the sample relative to the FGF-CX nucleotide sequence in said control sample indicates the subject has a tissue proliferation-associated disorder.

In a still further aspect, the invention provides method of treating or preventing or delaying a tissue proliferation-associated disorder, cancer, oral mucositis (also known as stomatitis), radiation sickness, oral candidiasis, inflammatory bowel disease, ischemic stroke, hemorrhagic stroke, trauma, spinal cord damage, heavy metal or toxin poisoning, neurodegenerative diseases (such as Alzheimer's, Parkinson's Disease, Amyotrophic Lateral Sclerosis, Huntington's Disease) rheumatoid arthritis and osteoarthritis.

The method includes administering to a subject in which such treatment or prevention or delay is desired a FGF-CX nucleic acid, a FGF-CX polypeptide, or a FGF-CX antibody in an amount sufficient to treat, prevent, or delay a tissue proliferation-associated disorder in the subject.

The tissue proliferation-associated disorders diagnosed, treated, prevented or delayed using the FGF-CX nucleic acid molecules, polypeptides or antibodies can involve epithelial cells, e.g., fibroblasts and keratinocytes in the anterior eye after surgery. Other tissue proliferation-associated disorder include, e.g., tumors, restenosis, psoriasis, Dupuytren's contracture, diabetic complications, Kaposi sarcoma, and rheumatoid arthritis.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### BRIEF DESCRIPTION OF THE DRAWINGS

FIG. 1: Dose Response of CG53135-induced DNA synthesis in NIH 3T3 Fibroblasts. Serum starved NIH 3T3 cells were treated with purified CG53135-01 (CG53135 in figure), 10% serum or vehicle only (control). DNA synthesis was measured in triplicate for each sample, using a BrdU incorporation assay. Data points represent average BrdU incorporation and bars represent standard error (SE).

FIG. 2: CG53135 stimulates Growth of NIH 3T3 Fibroblasts. Duplicate wells of serum starved NIH 3T3 cells were treated for 1 day with purified CG53135-01 (1 ug) or vehicle control. Cell counts for each well were determined in duplicate. Y-axis identifies cell number, which is the average of 4 cell counts (treatment duplicates×duplicate counts) and standard error (SE).

FIG. 3: CG53135 induces DNA synthesis in 786-O Kidney Epithelial cells. Serum starved 786-O cells were left untreated or treated with partially purified CG53135-01 (from 5 ng/uL stock), or with vehicle control (mock). DNA synthesis was measured in triplicate for each sample, using a BrdU incorporation assay. Data points represent average BrdU incorporation and bars represent standard error (SE).

FIG. 4. Effect of CG53 135-05 in the treatment of radiation-induced mucositis. The total number of days in which animals in each group exhibited a mucositis score ≧3 was summed and expressed as a percentage of the total number of days scored. Statistical significance of observed differences with the respective vehicle control was calculated using chi-square analysis.

FIG. 5. Effect of Mucositis on the duration of mucositis induced by chemotherapy. The number of days with mucositis scores ≧3 was evaluated. To examine the levels of clinically significant mucositis as defined by presentiation with open ulcers (score ≧3), the total number of days in which an animal exhibited an elevated score was summed and expressed as a percentage of the total number of days scored for each group. Statistical significance of observed differences was calculated using Chi-square analysis. Vehicle control=disease control.

### DETAILED DESCRIPTION OF THE INVENTION

The invention is based in part on the discovery of FGF-CX nucleic acid sequence, which encodes a polypeptide that is a member of the fibroblast growth factor family and their method of use thereof.

Fibroblast Growth Factors

The fibroblast growth factor (FGF) group of cytokines includes at least 23 members that regulate diverse cellular functions such as growth, survival, apoptosis, motility and differentiation. These molecules transduce signals via high affinity interactions with cell surface tyrosine kinase FGF receptors (FGFRs). FGF receptors are expressed on most types of cells in tissue culture. Dimerization of FGF receptor monomers upon ligand binding has been reported to be a requisite for activation of the kinase domains, leading to receptor trans phosphorylation. FGF receptor-1 (FGFR-1), which shows the broadest expression pattern of the four FGF receptors, contains at least seven tyrosine phosphorylation sites. A number of signal transduction molecules are affected by binding with different affinities to these phosphorylation sites.

In addition to participating in normal growth and development, known FGFs have also been implicated in the generation of pathological states, including cancer. FGFs may contribute to malignancy by directly enhancing the growth of tumor cells. For example, autocrine growth stimulation through the co-expression of FGF and FGFR in the same cell has been reported to lead to cellular transformation.

Previously described members of the FGF family regulate diverse cellular functions such as growth, survival, apoptosis, motility and differentiation (Szebenyi & Fallon (1999) Int. Rev. Cytol. 185, 45-106). These molecules transduce signals intracellularly via high affinity interactions with cell surface tyrosine kinase FGF receptors (FGFRs), four of which have been identified to date (Xu et al. (1999) Cell Tissue Res. 296, 33-43; Klint & Claesson-Welsh (1999) Front. Biosci. 4, 165-177). These FGF receptors are expressed on most types of cells in tissue culture. Dimerization of FGF receptor monomers upon ligand binding has been reported to be a requisite for activation of the kinase domains, leading to receptor trans phosphorylation. FGF receptor-1 (FGFR-1), which shows the broadest expression pattern of the four FGF receptors, contains at least seven tyrosine phosphorylation sites. A number of signal transduction molecules are affected by binding with different affinities to these phosphorylation sites.

FGFs also bind, albeit with low affinity, to heparin sulfate proteoglycans (HSPGs) present on most cell surfaces and extracellular matrices (ECM). Interactions between FGFs and HSPGs serve to stabilize FGF/FGFR interactions, and to sequester FGFs and protect them from degradation (Szebenyi. & Fallon (1999)). Due to its growth-promoting capabilities, one member of the FGF family, FGF-7, is currently in clinical trials for the treatment of chemotherapy-induced mucositis (Danilenko (1999) Toxicol. Pathol. 27, 64-71).

In addition to participating in normal growth and development, known FGFs have also been implicated in the generation of pathological states, including cancer (Basilico & Moscatelli (1992) Adv. Cancer Res. 59, 115-165). FGFs may contribute to malignancy by directly enhancing the growth of tumor cells. For example, autocrine growth stimulation through the co-expression of FGF and FGFR in the same cell leads to cellular transformation (Matsumoto-Yoshitomi, et al., (1997) Int. J. Cancer 71, 442-450). Likewise, the constitutive activation of FGFR via mutation or rearrangement leads to uncontrolled proliferation (Lorenzi, et al., (1996) Proc. Natl. Acad. Sci. USA. 93, 8956-8961; Li, et al., (1997) Oncogene 14, 1397-1406). Furthermore, some FGFs are angiogenic (Gerwins, et al., (2000) Crit. Rev. Oncol. Hematol. 34, 185-194). Such FGFs may contribute to the tumorigenic process by facilitating the development of the blood supply needed to sustain tumor growth. Not surprisingly, at least one FGF is currently under investigation as a potential target for cancer therapy (Gasparini (1999) Drugs 58, 17-38).

Expression of FGFs and their receptors in the brains of perinatal and adult mice has been examined. Messenger RNA all FGF genes, with the exception of FGF-4, is detected in these tissues. FGF-3, FGF-6, FGF-7 and FGF-8 genes demonstrate higher expression in the late embryonic stages than in postnatal stages, suggesting that these members are involved in the late stages of brain development. In contrast, expression of FGF-1 and FGF-5 increased after birth. In particular, FGF-6 expression in perinatal mice has been reported to be restricted to the central nervous system and skeletal muscles, with intense signals in the developing cerebrum in embryos but in cerebellum in 5-day-old neonates. FGF-receptor (FGFR)-4, a cognate receptor for FGF-6, demonstrate similar spatiotemporal expression, suggesting that FGF-6 and FGFR-4 plays significant roles in the maturation of nervous system as a ligand-receptor system. According to Ozawa et al., these results strongly suggest that the various FGFs and their receptors are involved in the regulation of a variety of developmental processes of brain, such as proliferation and migration of neuronal progenitor cells, neuronal and glial differentiation, neurite extensions, and synapse formation.

Glia-activating factor (“GAF”), another FGF family member, is a heparin-binding growth factor that was purified from the culture supernatant of a human glioma cell line. See, Miyamoto et al., 1993, Mol Cell Biol 13(7): 4251-4259. GAF shows a spectrum of activity slightly different from those of other known growth factors, and is designated as FGF-9. The human FGF-9 cDNA encodes a polypeptide of 208 amino acids. Sequence similarity to other members of the FGF family was estimated to be around 30%. Two cysteine residues and other consensus sequences found in other family members were also well conserved in the FGF-9 sequence. FGF-9 was found to have no typical signal sequence in its N terminus like those in acidic FGF and basic FGF.

Acidic FGF and basic FGF are known not to be secreted from cells in a conventional manner. However, FGF-9 was found to be secreted efficiently from cDNA-transfected COS cells despite its lack of a typical signal sequence. It could be detected exclusively in the culture medium of cells. The secreted protein lacked no amino acid residues at the N terminus with respect to those predicted by the cDNA sequence, except the initiation methionine. The rat FGF-9 cDNA was also cloned, and the structural analysis indicated that the FGF-9 gene is highly conserved.

Section I

Included within the invention are FGF-CX nucleic acids, isolated nucleic acids that encode FGF-CX polypeptide or a portion thereof, FGF-CX polypeptides, vectors containing these nucleic acids, host cells transformed with the FGF-CX nucleic acids, anti-FGF-CX antibodies, and pharmaceutical compositions. Also disclosed are methods of making FGF-CX polypeptides, as well as methods of screening, diagnosing, treating conditions using these compounds, and methods of screening compounds that modulate FGF-CX polypeptide activity. Table A provides a summary of the FGF-CX nucleic acids and their encoded polypeptides.

TABLE A

| | | SEQ ID | SEQ ID | |
|---|---|---|---|---|
| | | NO | NO | |
| FGF-CXX | Internal | (nucleic | (amino | |
| Assignment | Identification | acid) | acid) | Homology |
| FGF-CX1a | CG53135-05 | 1 | 2 | Fibroblast growth |
| | | | | factor-20 (FGF-20) - |
| | | | | Homo sapiens |
| FGF-CX1b | CG53135-01 | 3 | 4 | Fibroblast growth |
| | | | | factor-20 (FGF-20) - |
| | | | | Homo sapiens |
| FGF-CX1c | CG53135-04 | 5 | 6 | Fibroblast growth |
| | | | | factor-20 (FGF-20) - |
| | | | | Homo sapiens |
| FGF-CX1d | 250059596 | 7 | 8 | Fibroblast growth |
| | | | | factor-20 (FGF-20) - |
| | | | | Homo sapiens |
| FGF-CX1e | 250059629 | 9 | 10 | Fibroblast growth |
| | | | | factor-20 (FGF-20) - |
| | | | | Homo sapiens |
| FGF-CX1f | 250059669 | 11 | 12 | Fibroblast growth |
| | | | | factor-20 (FGF-20) - |
| | | | | Homo sapiens |
| FGF-CX1g | 316351224 | 13 | 14 | Fibroblast growth |
| | | | | factor-20 (FGF-20) - |
| | | | | Homo sapiens |
| FGF-CX1h | 317459553 | 15 | 16 | Fibroblast growth |
| | | | | factor-20 (FGF-20) - |
| | | | | Homo sapiens |
| FGF-CX1i | 317459571 | 17 | 18 | Fibroblast growth |
| | | | | factor-20 (FGF-20) - |
| | | | | Homo sapiens |
| FGF-CX1j | CG53135-02 | 19 | 20 | Fibroblast growth |
| | | | | factor-20 (FGF-20) - |
| | | | | Homo sapiens |
| FGF-CX1k | CG53135-03 | 21 | 22 | Fibroblast growth |
| | | | | factor-20 (FGF-20) - |
| | | | | Homo sapiens |
| FGF-CX1l | CG53135-06 | 23 | 24 | Fibroblast growth |
| | | | | factor-20 (FGF-20) - |
| | | | | Homo sapiens |
| FGF-CX1m | CG53135-07 | 25 | 26 | Fibroblast growth |
| | | | | factor-20 (FGF-20) - |
| | | | | Homo sapiens |
| FGF-CX1n | CG53135-08 | 27 | 28 | Fibroblast growth |
| | | | | factor-20 (FGF-20) - |
| | | | | Homo sapiens |
| FGF-CX1o | CG53135-09 | 29 | 30 | Fibroblast growth |
| | | | | factor-20 (FGF-20) - |
| | | | | Homo sapiens |
| FGF-CX1p | CG53135-10 | 31 | 32 | Fibroblast growth |
| | | | | factor-20 (FGF-20) - |
| | | | | Homo sapiens |
| FGF-CX1q | CG53135-11 | 33 | 34 | Fibroblast growth |
| | | | | factor-20 (FGF-20) - |
| | | | | Homo sapiens |
| FGF-CX1r | CG53135-12 | 35 | 36 | Fibroblast growth |
| | | | | factor-20 (FGF-20) - |
| | | | | Homo sapiens |

One aspect of the invention pertains to isolated nucleic acid molecules that encode FGF-CX polypeptides or biologically active portions thereof. Also included in the invention are nucleic acid fragments sufficient for use as hybridization probes to identify FGF-CX-encoding nucleic acids (e.g., FGF-CX mRNAs) and fragments for use as PCR primers for the amplification and/or mutation of FGF-CX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised double-stranded DNA.

An FGF-CX nucleic acid can encode a mature FGF-CX polypeptide. As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full-length gene product, encoded by the corresponding gene. Alternatively, it may be defined as the polypeptide, precursor or proprotein encoded by an ORF described herein. The product “mature” form arises, again by way of nonlimiting example, as a result of one or more naturally occurring processing steps as they may take place within the cell, or host cell, in which the gene product arises. Examples of such processing steps leading to a “mature” form of a polypeptide or protein include the cleavage of the N-terminal methionine residue encoded by the initiation codon of an ORF, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them.

The term “probes”, as utilized herein, refers to nucleic acid sequences of variable length, preferably between at least about 10 nucleotides (nt), 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes may be single- or double-stranded and designed to have specificity in PCR, membrane-based hybridization technologies, or ELISA-like technologies.

The term “isolated” nucleic acid molecule, as utilized herein, is one, which is separated from other nucleic acid molecules which are present in the natural source of the nucleic acid. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated FGF-CX nucleic acid molecules can contain less than about 5 kb, 4 kb, 3 kb, 2 kb, 1 kb, 0.5 kb or 0.1 kb of nucleotide sequences which naturally flank the nucleic acid molecule in genomic DNA of the cell/tissue from which the nucleic acid is derived (e.g., brain, heart, liver, spleen, etc.). Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the invention, e.g., a nucleic acid molecule having the nucleotide sequence of SEQ ID NOs: 1, 3, 5, 7, 9, 11, 13, 15, 17, 19, 21, 23, 25, 27, 29, 31, 33, 35, or a complement of this aforementioned nucleotide sequence, can be isolated using standard molecular biology techniques and the sequence information provided herein. Using all or a portion of the nucleic acid sequence of SEQ ID NOs: 1, 3, 5, 7, 9, 11, 13, 15, 17, 19, 21, 23, 25, 27, 29, 31, 33, and 35, as a hybridization probe, FGF-CX molecules can be isolated using standard hybridization and cloning techniques (e.g., as described in Sambrook, et al., (eds.), MOLECULAR CLONING: A LABORATORY MANUAL 2^{nd} Ed., Cold Spring Harbor Laboratory Press, Cold Spring Harbor, N.Y., 1989; and Ausubel, et al., (eds.), CURRENT PROTOCOLS IN MOLECULAR BIOLOGY, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to FGF-CX nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides of SEQ ID NOs: 1, 3, 5, 7, 9, 11, 13, 15, 17, 19, 21, 23, 25, 27, 29, 31, 33, and 35, or a complement thereof. Oligonucleotides may be chemically synthesized and may also be used as probes.

In another embodiment, an isolated nucleic acid molecule of the invention comprises a nucleic acid molecule that is a complement of the nucleotide sequence shown in SEQ IDNOs: 1, 3, 5, 7, 9, 11, 13, 15, 17, 19, 21, 23, 25, 27, 29, 31, 33, and 35, or a portion of this nucleotide sequence (e.g., a fragment that can be used as a probe or primer or a fragment encoding a biologically-active portion of an FGF-CX polypeptide). A nucleic acid molecule that is complementary to the nucleotide sequence shown in SEQ ID NOs: 1, 3, 5, 7, 9, 11, 13, 15, 17, 19, 21, 23, 25, 27, 29, 31, 33, and 35, is one that is sufficiently complementary to the nucleotide sequence shown in SEQ ID NOs: 1, 3, 5, 7, 9, 11, 13, 15, 17, 19, 21, 23, 25, 27, 29, 31, 33, and 35, that it can hydrogen bond with little or no mismatches to the nucleotide sequence shown SEQ ID NOs: 1, 3, 5, 7, 9, 11, 13, 15, 17, 19, 21, 23, 25, 27, 29, 31, 33, and 35, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type. Homologs are nucleic acid sequences or amino acid sequences of a particular gene that are derived from different species.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of FGF-CX polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for an FGF-CX polypeptide of species other than humans, including, but not limited to: vertebrates, and thus can include, e.g., frog, mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding human FGF-CX protein. Homologous nucleic acid sequences include those nucleic acid sequences that encode conservative amino acid substitutions (see below) in SEQ ID NOs: 1, 3, 5, 7, 9, 11, 13, 15, 17, 19, 21, 23, 25, 27, 29, 31, 33, and 35, as well as a polypeptide possessing FGF-CX biological activity. Various biological activities of the FGF-CX proteins are described below.

As used herein, “identical” residues correspond to those residues in a comparison between two sequences where the equivalent nucleotide base or amino acid residue in an alignment of two sequences is the same residue. Residues are alternatively described as “similar” or “positive” when the comparisons between two sequences in an alignment show that residues in an equivalent position in a comparison are either the same amino acid or a conserved amino acid as defined below.

An FGF-CX polypeptide is encoded by the open reading frame (“ORF”) of an FGF-CX nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bonafide cellular protein, a minimum size requirement is often set, e.g., a stretch of DNA that would encode a protein of 50 amino acids or more.

The nucleotide sequences determined from the cloning of the human FGF-CX genes allows for the generation of probes and primers designed for use in identifying and/or cloning FGF-CX homologues in other cell types, e.g. from other tissues, as well as FGF-CX homologues from other vertebrates. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence of SEQ ID NOs: 1, 3, 5, 7, 9, 11, 13, 15, 17, 19, 21, 23, 25, 27, 29, 31, 33, and 35; or an anti-sense strand nucleotide sequence of SEQ ID NOs: 1, 3, 5, 7, 9, 11, 13, 15, 17, 19, 21, 23, 25, 27, 29, 31, 33, and 35; or of a naturally occurring mutant of SEQ ID NOs: 1, 3, 5, 7, 9, 11, 13, 15, 17, 19, 21, 23, 25, 27, 29, 31, 33, and 35.

Probes based on the human FGF-CX nucleotide sequences can be used to detect transcripts or genomic sequences encoding the same or homologous proteins. In various embodiments, the probe further comprises a label group attached thereto, e.g. the label group can be a radioisotope, a fluorescent compound, an enzyme, or an enzyme co-factor. Such probes can be used as a part of a diagnostic test kit for identifying cells or tissues which mis-express an FGF-CX protein, such as by measuring a level of an FGF-CX-encoding nucleic acid in a sample of cells from a subject e.g., detecting FGF-CX mRNA levels or determining whether a genomic FGF-CX gene has been mutated or deleted.

“A polypeptide having a biologically-active portion of an FGF-CX polypeptide” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of FGF-CX” can be prepared by isolating a portion SEQ ID NOs: 1, 3, 5, 7, 9, 11, 13, 15, 17, 19, 21, 23, 25, 27, 29, 31, 33, and 35, that encodes a polypeptide having an FGF-CX biological activity (the biological activities of the FGF-CX proteins are described below), expressing the encoded portion of FGF-CX protein (e.g., by recombinant expression in vitro) and assessing the activity of the encoded portion of FGF-CX.

FGF-CX Nucleic Acid and Polypeptide Variants

The invention further encompasses nucleic acid molecules that differ from the nucleotide sequences shown SEQ ID NOs: 1, 3, 5, 7, 9, 11, 13, 15, 17, 19, 21, 23, 25, 27, 29, 31, 33, and 35, due to degeneracy of the genetic code and thus encode the same FGF-CX proteins as that encoded by the nucleotide sequences shown in SEQ ID NOs: 1, 3, 5, 7, 9, 11, 13, 15, 17, 19, 21, 23, 25, 27, 29, 31, 33, and 35. In another embodiment, an isolated nucleic acid molecule of the invention has a nucleotide sequence encoding a protein having an amino acid sequence shown in SEQ ID NOs: 2, 4, 6, 8, 10, 12, 14, 16, 18, 20, 22, 24, 26, 28, 30, 32, 34, 36.

In addition to the human FGF-CX nucleotide sequences shown in SEQ ID NOs: 1, 3, 5, 7, 9, 11, 13, 15, 17, 19, 21, 23, 25, 27, 29, 31, 33, and 35, it will be appreciated by those skilled in the art that DNA sequence polymorphisms that lead to changes in the amino acid sequences of the FGF-CX polypeptides may exist within a population (e.g., the human population). Such genetic polymorphism in the FGF-CX genes may exist among individuals within a population due to natural allelic variation. As used herein, the terms “gene” and “recombinant gene” refer to nucleic acid molecules comprising an open reading frame (ORF) encoding an FGF-CX protein, preferably a vertebrate FGF-CX protein. Such natural allelic variations can typically result in 1-5% variance in the nucleotide sequence of the FGF-CX genes. Any and all such nucleotide variations and resulting amino acid polymorphisms in the FGF-CX polypeptides, which are the result of natural allelic variation and that do not alter the functional activity of the FGF-CX polypeptides, are intended to be within the scope of the invention.

Moreover, nucleic acid molecules encoding FGF-CX proteins from other species, and thus that have a nucleotide sequence that differs from the human sequence SEQ ID NOs: 1, 3, 5, 7, 9, 11, 13, 15, 17, 19, 21, 23, 25, 27, 29, 31, 33, and 35, are intended to be within the scope of the invention. Nucleic acid molecules corresponding to natural allelic variants and homologues of the FGF-CX cDNAs of the invention can be isolated based on their homology to the human FGF-CX nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid molecule comprising the nucleotide sequence of SEQ ID NOs: 1, 3, 5, 7, 9, 11, 13, 15, 17, 19, 21, 23, 25, 27, 29, 31, 33, and 35, In another embodiment, the nucleic acid is at least 10, 25, 50, 100, 250, 500, 750, 1000, 1500, or 2000 or more nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other. Homologs (i.e., nucleic acids encoding FGF-CX proteins derived from species other than human) or other related sequences (e.g. paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel, et al., (eds.), CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C. An isolated nucleic acid molecule of the invention that hybridizes under stringent conditions to the sequences of SEQ ID NOs: 1, 3, 5, 7, 9, 11, 13, 15, 17, 19, 21, 23, 25, 27, 29, 31, 33, and 35, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NOs: 1, 3, 5, 7, 9, 11, 13, 15, 17, 19, 21, 23, 25, 27, 29, 31, 33, and 35, or fragments, analogs or derivatives thereof, under conditions of moderate stringency is provided. A non-limiting example of moderate stringency hybridization conditions are hybridization in 6×SSC, 5× Denhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well-known within the art. See, e.g., Ausubel, et al. (eds.), 1993, CURRENT PROTOCOLS IN MOLECULAR BIOLOGY, John Wiley & Sons, N.Y., and Kriegler, 1990; GENE TRANSFER AND EXPRESSION, A LABORATORY MANUAL, Stockton Press, N.Y.

In a third embodiment, a nucleic acid that is hybridizable to the nucleic acid molecule comprising the nucleotide sequences of SEQ ID NOs: 1, 3, 5, 7, 9, 11, 13, 15, 17, 19, 21, 23, 25, 27, 29, 31, 33, and 35, or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel, et al. (eds.), 1993, CURRENT PROTOCOLS IN MOLECULAR BIOLOGY, John Wiley & Sons, N.Y., and Kriegler, 1990, GENE TRANSFER AND EXPRESSION, A LABORATORY MANUAL, Stockton Press, N.Y.; Shilo and Weinberg, 1981. Proc Natl Acad Sci USA 78: 6789-6792.

Conservative Mutations

In addition to naturally-occurring allelic variants of FGF-CX sequences that may exist in the population, the skilled artisan will further appreciate that changes can be introduced by mutation into the nucleotide sequences of SEQ ID NOs: 1, 3, 5, 7, 9, 11, 13, 15, 17, 19, 21, 23, 25, 27, 29, 31, 33, and 35, thereby leading to changes in the amino acid sequences of the encoded FGF-CX proteins, without altering the functional ability of said FGF-CX proteins. For example, nucleotide substitutions leading to amino acid substitutions at “non-essential” amino acid residues can be made in the sequence of SEQ ID NOs: 2, 4, 6, 8, 10, 12, 14, 16, 18, 20, 22, 24, 26, 28, 30, 32, 34, 36. A “non-essential” amino acid residue is a residue that can be altered from the wild-type sequences of the FGF-CX proteins without altering their biological activity, whereas an “essential” amino acid residue is required for such biological activity. For example, amino acid residues that are conserved among the FGF-CX proteins of the invention are predicted to be particularly non-amenable to alteration. Amino acids for which conservative substitutions can be made are well-known within the art.

Another aspect of the invention pertains to nucleic acid molecules encoding FGF-CX proteins that contain changes in amino acid residues that are not essential for activity. Such FGF-CX proteins differ in amino acid sequence from SEQ ID NOs: 2, 4, 6, 8, 10, 12, 14, 16, 18, 20, 22, 24, 26, 28, 30, 32, 34, 36, yet retain biological activity. In one embodiment, the isolated nucleic acid molecule comprises a nucleotide sequence encoding a protein, wherein the protein comprises an amino acid sequence at least about 45% homologous to the amino acid sequences of SEQ ID NOs: 2, 4, 6, 8, 10, 12, 14, 16, 18, 20, 22, 24, 26, 28, 30, 32, 34, 36. Preferably, the protein encoded by the nucleic acid molecule is at least about 60% homologous to SEQ ID NOs: 2, 4, 6, 8, 10, 12, 14, 16, 18, 20, 22, 24, 26, 28, 30, 32, 34, 36; more preferably at least about 70% homologous to SEQ ID NOs: 2, 4, 6, 8, 10, 12, 14, 16, 18, 20, 22, 24, 26, 28, 30, 32, 34, 36; still more preferably at least about 80% homologous to SEQ ID NOs: 2, 4, 6, 8, 10, 12, 14, 16, 18, 20, 22, 24, 26, 28, 30, 32, 34, 36; even more preferably at least about 90% homologous to SEQ ID NOs: 2, 4, 6, 8, 10, 12, 14, 16, 18, 20, 22, 24, 26, 28, 30, 32, 34, 36; and most preferably at least about 95% homologous to SEQ ID NOs: 2, 4, 6, 8, 10, 12, 14, 16, 18, 20, 22, 24, 26, 28, 30, 32, 34, 36.

An isolated nucleic acid molecule encoding an FGF-CX protein homologous to the protein of SEQ ID NOs: 2, 4, 6, 8, 10, 12, 14, 16, 18, 20, 22, 24, 26, 28, 30, 32, 34, 36, can be created by introducing one or more nucleotide substitutions, additions or deletions into the nucleotide sequence of SEQ ID NOs: 1, 3, 5, 7, 9, 11, 13, 15, 17, 19, 21, 23, 25, 27, 29, 31, 33, and 35, such that one or more amino acid substitutions, additions or deletions are introduced into the encoded protein.

Mutations can be introduced into SEQ ID NOs: 2, 4, 6, 8, 10, 12, 14, 16, 18, 20, 22, 24, 26, 28, 30, 32, 34, 36, by standard techniques, such as site-directed mutagenesis and PCR-mediated mutagenesis. Preferably, conservative amino acid substitutions are made at one or more predicted, non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in the FGF-CX protein is replaced with another amino acid residue from the same side chain family. Alternatively, in another embodiment, mutations can be introduced randomly along all or part of an FGF-CX coding sequence, such as by saturation mutagenesis, and the resultant mutants can be screened for FGF-CX biological activity to identify mutants that retain activity. Following mutagenesis of SEQ ID NOs: 1, 3, 5, 7, 9, 11, 13, 15, 17, 19, 21, 23, 25, 27, 29, 31, 33, and 35, the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, VLIM, HFY, wherein the letters within each group represent the single letter amino acid code.

In one embodiment, a mutant FGF-CX protein can be assayed for (i) the ability to form protein:protein interactions with other FGF-CX proteins, other cell-surface proteins, or biologically-active portions thereof, (ii) complex formation between a mutant FGF-CX protein and an FGF-CX ligand; or (iii) the ability of a mutant FGF-CX protein to bind to an intracellular target protein or biologically-active portion thereof, (e.g. avidin proteins).

In yet another embodiment, a mutant FGF-CX protein can be assayed for the ability to regulate a specific biological function (e.g., regulation of insulin release).

Interfering RNA

In one aspect of the invention, FGF-CX gene expression can be attenuated by RNA interference. One approach well-known in the art is short interfering RNA (siRNA) mediated gene silencing where expression products of a FGF-CX gene are targeted by specific double stranded FGF-CX derived siRNA nucleotide sequences that are complementary to at least a 19-25 nt long segment of the FGF-CX gene transcript, including the 5′ untranslated (UT) region, the ORF, or the 3′ UT region. See, e.g., PCT applications WO00/44895, WO99/32619, WO01/75164, WO01/92513, WO 01/29058, WO01/89304, WO02/16620, and WO02/29858, each incorporated by reference herein in their entirety. Targeted genes can be a FGF-CX gene, or an upstream or downstream modulator of the FGF-CX gene. Nonlimiting examples of upstream or downstream modulators of a FGF-CX gene include, e.g., a transcription factor that binds the FGF-CX gene promoter, a kinase or phosphatase that interacts with a FGF-CX polypeptide, and polypeptides involved in a FGF-CX regulatory pathway.

According to the methods of the present invention, FGF-CX gene expression is silenced using short interfering RNA. A FGF-CX polynucleotide according to the invention includes a siRNA polynucleotide. Such a FGF-CX siRNA can be obtained using a FGF-CX polynucleotide sequence, for example, by processing the FGF-CX ribopolynucleotide sequence in a cell-free system, such as but not limited to a Drosophila extract, or by transcription of recombinant double stranded FGF-CX RNA or by chemical synthesis of nucleotide sequences homologous to a FGF-CX sequence. See, e.g., Tuschl, Zamore, Lehmann, Bartel and Sharp (1999), Genes & Dev. 13: 3191-3197, incorporated herein by reference in its entirety. When synthesized, a typical 0.2 micromolar-scale RNA synthesis provides about 1 milligram of siRNA, which is sufficient for 1000 transfection experiments using a 24-well tissue culture plate format.

The most efficient silencing is generally observed with siRNA duplexes composed of a 21-nt sense strand and a 21-nt antisense strand, paired in a manner to have a 2-nt 3′ overhang. The sequence of the 2-nt 3′ overhang makes an additional small contribution to the specificity of siRNA target recognition. The contribution to specificity is localized to the unpaired nucleotide adjacent to the first paired bases. In one embodiment, the nucleotides in the 3′ overhang are ribonucleotides. In an alternative embodiment, the nucleotides in the 3′ overhang are deoxyribonucleotides. Using 2′-deoxyribonucleotides in the 3′ overhangs is as efficient as using ribonucleotides, but deoxyribonucleotides are often cheaper to synthesize and are most likely more nuclease resistant.

A contemplated recombinant expression vector of the invention comprises a FGF-CX DNA molecule cloned into an expression vector comprising operatively-linked regulatory sequences flanking the FGF-CX sequence in a manner that allows for expression (by transcription of the DNA molecule) of both strands. An RNA molecule that is antisense to FGF-CX mRNA is transcribed by a first promoter (e.g., a promoter sequence 3′ of the cloned DNA) and an RNA molecule that is the sense strand for the FGF-CX mRNA is transcribed by a second promoter (e.g., a promoter sequence 5′ of the cloned DNA). The sense and antisense strands may hybridize in vivo to generate siRNA constructs for silencing of the FGF-CX gene. Alternatively, two constructs can be utilized to create the sense and anti-sense strands of a siRNA construct. Finally, cloned DNA can encode a construct having secondary structure, wherein a single transcript has both the sense and complementary antisense sequences from the target gene or genes. In an example of this embodiment, a hairpin RNAi product is homologous to all or a portion of the target gene. In another example, a hairpin RNAi product is a siRNA. The regulatory sequences flanking the FGF-CX sequence may be identical or may be different, such that their expression may be modulated independently, or in a temporal or spatial manner.

In a specific embodiment, siRNAs are transcribed intracellularly by cloning the FGF-CX gene templates into a vector containing, e.g., a RNA pol III transcription unit from the smaller nuclear RNA (snRNA) U6 or the human RNase P RNA H1. One example of a vector system is the GeneSuppressor™ RNA Interference kit (commercially available from Imgenex). The U6 and H1 promoters are members of the type III class of Pol III promoters. The +1 nucleotide of the U6-like promoters is always guanosine, whereas the +1 for H1 promoters is adenosine. The termination signal for these promoters is defined by five consecutive thymidines. The transcript is typically cleaved after the second uridine. Cleavage at this position generates a 3′ UU overhang in the expressed siRNA, which is similar to the 3′ overhangs of synthetic siRNAs. Any sequence less than 400 nucleotides in length can be transcribed by these promoter, therefore they are ideally suited for the expression of around 21-nucleotide siRNAs in, e.g., an approximately 50-nucleotide RNA stem-loop transcript.

A siRNA vector appears to have an advantage over synthetic siRNAs where long term knock-down of expression is desired. Cells transfected with a siRNA expression vector would experience steady, long-term mRNA inhibition. In contrast, cells transfected with exogenous synthetic siRNAs typically recover from mRNA suppression within seven days or ten rounds of cell division. The long-term gene silencing ability of siRNA expression vectors may provide for applications in gene therapy.

In general, siRNAs are chopped from longer dsRNA by an ATP-dependent ribonuclease called DICER. DICER is a member of the RNase III family of double-stranded RNA-specific endonucleases. The siRNAs assemble with cellular proteins into an endonuclease complex. In vitro studies in Drosophila suggest that the siRNAs/protein complex (siRNP) is then transferred to a second enzyme complex, called an RNA-induced silencing complex (RISC), which contains an endoribonuclease that is distinct from DICER. RISC uses the sequence encoded by the antisense siRNA strand to find and destroy mRNAs of complementary sequence. The siRNA thus acts as a guide, restricting the ribonuclease to cleave only mRNAs complementary to one of the two siRNA strands.

A FGF-CX mRNA region to be targeted by siRNA is generally selected from a desired FGF-CX sequence beginning 50 to 100 nt downstream of the start codon. Alternatively, 5′ or 3′ UTRs and regions nearby the start codon can be used but are generally avoided, as these may be richer in regulatory protein binding sites. UTR-binding proteins and/or translation initiation complexes may interfere with binding of the siRNP or RISC endonuclease complex. An initial BLAST homology search for the selected siRNA sequence is done against an available nucleotide sequence library to ensure that only one gene is targeted. Specificity of target recognition by siRNA duplexes indicate that a single point mutation located in the paired region of an siRNA duplex is sufficient to abolish target mRNA degradation. See, Elbashir et al. 2001 EMBO J. 20(23):6877-88. Hence, consideration should be taken to accommodate SNPs, polymorphisms, allelic variants or species-specific variations when targeting a desired gene.

In one embodiment, a complete FGF-CX siRNA experiment includes the proper negative control. A negative control siRNA generally has the same nucleotide composition as the FGF-CX siRNA but lack significant sequence homology to the genome. Typically, one would scramble the nucleotide sequence of the FGF-CX siRNA and do a homology search to make sure it lacks homology to any other gene.

Two independent FGF-CX siRNA duplexes can be used to knock-down a target FGF-CX gene. This helps to control for specificity of the silencing effect. In addition, expression of two independent genes can be simultaneously knocked down by using equal concentrations of different FGF-CX siRNA duplexes, e.g., a FGF-CX siRNA and an siRNA for a regulator of a FGF-CX gene or polypeptide. Availability of siRNA-associating proteins is believed to be more limiting than target mRNA accessibility.

A targeted FGF-CX region is typically a sequence of two adenines (AA) and two thymidines (TT) divided by a spacer region of nineteen (N19) residues (e.g., AA(N19)TT). A desirable spacer region has a G/C-content of approximately 30% to 70%, and more preferably of about 50%. If the sequence AA(NI 9)TT is not present in the target sequence, an alternative target region would be AA(N21). The sequence of the FGF-CX sense siRNA corresponds to (N19)TT or N21, respectively. In the latter case, conversion of the 3′ end of the sense siRNA to TT can be performed if such a sequence does not naturally occur in the FGF-CX polynucleotide. The rationale for this sequence conversion is to generate a symmetric duplex with respect to the sequence composition of the sense and antisense 3′ overhangs. Symmetric 3′ overhangs may help to ensure that the siRNPs are formed with approximately equal ratios of sense and antisense target RNA-cleaving siRNPs. See, e.g., Elbashir, Lendeckel and Tuschl (2001). Genes & Dev. 15: 188-200, incorporated by reference herein in its entirely. The modification of the overhang of the sense sequence of the siRNA duplex is not expected to affect targeted mRNA recognition, as the antisense siRNA strand guides target recognition.

Alternatively, if the FGF-CX target mRNA does not contain a suitable AA(N21) sequence, one may search for the sequence NA(N21). Further, the sequence of the sense strand and antisense strand may still be synthesized as 5′ (Nl 9)TT, as it is believed that the sequence of the 3′-most nucleotide of the antisense siRNA does not contribute to specificity. Unlike antisense or ribozyme technology, the secondary structure of the target mRNA does not appear to have a strong effect on silencing. See, Harborth, et al. (2001) J. Cell Science 114: 4557-4565, incorporated by reference in its entirety.

Transfection of FGF-CX siRNA duplexes can be achieved using standard nucleic acid transfection methods, for example, OLIGOFECTAMINE Reagent (commercially available from Invitrogen). An assay for FGF-CX gene silencing is generally performed approximately 2 days after transfection. No FGF-CX gene silencing has been observed in the absence of transfection reagent, allowing for a comparative analysis of the wild-type and silenced FGF-CX phenotypes. In a specific embodiment, for one well of a 24-well plate, approximately 0.84 μg of the siRNA duplex is generally sufficient. Cells are typically seeded the previous day, and are transfected at about 50% confluence. The choice of cell culture media and conditions are routine to those of skill in the art, and will vary with the choice of cell type. The efficiency of transfection may depend on the cell type, but also on the passage number and the confluency of the cells. The time and the manner of formation of siRNA-liposome complexes (e.g. inversion versus vortexing) are also critical. Low transfection efficiencies are the most frequent cause of unsuccessful FGF-CX silencing. The efficiency of transfection needs to be carefully examined for each new cell line to be used. Preferred cell are derived from a mammal, more preferably from a rodent such as a rat or mouse, and most preferably from a human. Where used for therapeutic treatment, the cells are preferentially autologous, although non-autologous cell sources are also contemplated as within the scope of the present invention.

For a control experiment, transfection of 0.84 μg single-stranded sense FGF-CX siRNA will have no effect on FGF-CX silencing, and 0.84 μg antisense siRNA has a weak silencing effect when compared to 0.84 μg of duplex siRNAs. Control experiments again allow for a comparative analysis of the wild-type and silenced FGF-CX phenotypes. To control for transfection efficiency, targeting of common proteins is typically performed, for example targeting of lamin A/C or transfection of a CMV-driven EGFP-expression plasmid (e.g. commercially available from Clontech). In the above example, a determination of the fraction of lamin A/C knockdown in cells is determined the next day by such techniques as immunofluorescence, Western blot, Northern blot or other similar assays for protein expression or gene expression. Lamin A/C monoclonal antibodies may be obtained from Santa Cruz Biotechnology.

Depending on the abundance and the half life (or turnover) of the targeted FGF-CX polynucleotide in a cell, a knock-down phenotype may become apparent after 1 to 3 days, or even later. In cases where no FGF-CX knock-down phenotype is observed, depletion of the FGF-CX polynucleotide may be observed by immunofluorescence or Western blotting. If the FGF-CX polynucleotide is still abundant after 3 days, cells need to be split and transferred to a fresh 24-well plate for re-transfection. If no knock-down of the targeted protein is observed, it may be desirable to analyze whether the target mRNA (FGF-CX upstream or a FGF-CX downstream gene) was effectively destroyed by the transfected siRNA duplex. Two days after transfection, total RNA is prepared, reverse transcribed using a target-specific primer, and PCR-amplified with a primer pair covering at least one exon-exon junction in order to control for amplification of pre-mRNAs. RT/PCR of a non-targeted mRNA is also needed as control. Effective depletion of the mRNA yet undetectable reduction of target protein may indicate that a large reservoir of stable FGF-CX protein may exist in the cell. Multiple transfection in sufficiently long intervals may be necessary until the target protein is finally depleted to a point where a phenotype may become apparent. If multiple transfection steps are required, cells are split 2 to 3 days after transfection. The cells may be transfected immediately after splitting.

An inventive therapeutic method of the invention contemplates administering a FGF-CX siRNA construct as therapy to compensate for increased or aberrant FGF-CX expression or activity. The FGF-CX ribopolynucleotide is obtained and processed into siRNA fragments, or a FGF-CX siRNA is synthesized, as described above. The FGF-CX siRNA is administered to cells or tissues using known nucleic acid transfection techniques, as described above. A FGF-CX siRNA specific for a FGF-CX gene will decrease or knockdown FGF-CX transcription products, which will lead to reduced FGF-CX polypeptide production, resulting in reduced FGF-CX polypeptide activity in the cells or tissues.

The present invention also encompasses a method of treating a disease or condition associated with the presence of a FGF-CX protein in an individual comprising administering to the individual an RNAi construct that targets the mRNA of the protein (the mRNA that encodes the protein) for degradation. A specific RNAi construct includes a siRNA or a double stranded gene transcript that is processed into siRNAs. Upon treatment, the target protein is not produced or is not produced to the extent it would be in the absence of the treatment.

Where the FGF-CX gene function is not correlated with a known phenotype, a control sample of cells or tissues from healthy individuals provides a reference standard for determining FGF-CX expression levels. Expression levels are detected using the assays described, e.g., RT-PCR, Northern blotting, Western blotting, ELISA, and the like. A subject sample of cells or tissues is taken from a mammal, preferably a human subject, suffering from a disease state. The FGF-CX ribopolynucleotide is used to produce siRNA constructs, that are specific for the FGF-CX gene product. These cells or tissues are treated by administering FGF-CX siRNA's to the cells or tissues by methods described for the transfection of nucleic acids into a cell or tissue, and a change in FGF-CX polypeptide or polynucleotide expression is observed in the subject sample relative to the control sample, using the assays described. This FGF-CX gene knockdown approach provides a rapid method for determination of a FGF-CX minus (FGF-CX⁻) phenotype in the treated subject sample. The FGF-CX⁻ phenotype observed in the treated subject sample thus serves as a marker for monitoring the course of a disease state during treatment.

In specific embodiments, a FGF-CX siRNA is used in therapy. Methods for the generation and use of a FGF-CX siRNA are known to those skilled in the art. Example techniques are provided below.

Production of RNAs

Sense RNA (ssRNA) and antisense RNA (asRNA) of FGF-CX are produced using known methods such as transcription in RNA expression vectors. In the initial experiments, the sense and antisense RNA are about 500 bases in length each. The produced ssRNA and asRNA (0.5 μM) in 10 mM Tris-HCl (pH 7.5) with 20 mM NaCl were heated to 95° C. for 1 min then cooled and annealed at room temperature for 12 to 16 h. The RNAs are precipitated and resuspended in lysis buffer (below). To monitor annealing, RNAs are electrophoresed in a 2% agarose gel in TBE buffer and stained with ethidium bromide. See, e.g., Sambrook et al., Molecular Cloning. Cold Spring Harbor Laboratory Press, Plainview, N.Y. (1989).

Lysate Preparation

Untreated rabbit reticulocyte lysate (Ambion) are assembled according to the manufacturer's directions. dsRNA is incubated in the lysate at 30° C. for 10 min prior to the addition of mRNAs. Then FGF-CX mRNAs are added and the incubation continued for an additional 60 min. The molar ratio of double stranded RNA and mRNA is about 200:1. The FGF-CX mRNA is radiolabeled (using known techniques) and its stability is monitored by gel electrophoresis.

In a parallel experiment made with the same conditions, the double stranded RNA is internally radiolabeled with a ³²P-ATP. Reactions are stopped by the addition of 2× proteinase K buffer and deproteinized as described previously (Tuschl et al., Genes Dev., 13:3191-3197 (1999)). Products are analyzed by electrophoresis in 15% or 18% polyacrylamide sequencing gels using appropriate RNA standards. By monitoring the gels for radioactivity, the natural production of 10 to 25 nt RNAs from the double stranded RNA can be determined.

The band of double stranded RNA, about 21-23 bps, is eluded. The efficacy of these 21-23 mers for suppressing FGF-CX transcription is assayed in vitro using the same rabbit reticulocyte assay described above using 50 nanomolar of double stranded 21-23 mer for each assay. The sequence of these 21-23 mers is then determined using standard nucleic acid sequencing techniques.

RNA Preparation

21 nt RNAs, based on the sequence determined above, are chemically synthesized using Expedite RNA phosphoramidites and thymidine phosphoramidite (Proligo, Germany). Synthetic oligonucleotides are deprotected and gel-purified (Elbashir, Lendeckel, & Tuschl, Genes & Dev. 15, 188-200 (2001)), followed by Sep-Pak C18 cartridge (Waters, Milford, Mass., USA) purification (Tuschl, et al., Biochemistry, 32:11658-11668 (1993)).

These RNAs (20 μM) single strands are incubated in annealing buffer (100 mM potassium acetate, 30 mM HEPES-KOH at pH 7.4, 2 mM magnesium acetate) for 1 min at 90° C. followed by 1 h at 37° C.

Cell Culture

A cell culture known in the art to regularly express FGF-CX is propagated using standard conditions. 24 hours before transfection, at approx. 80% confluency, the cells are trypsinized and diluted 1:5 with fresh medium without antibiotics (1-3×105 cells/ml) and transferred to 24-well plates (500 ml/well). Transfection is performed using a commercially available lipofection kit and FGF-CX expression is monitored using standard techniques with positive and negative control. A positive control is cells that naturally express FGF-CX while a negative control is cells that do not express FGF-CX. Base-paired 21 and 22 nt siRNAs with overhanging 3′ ends mediate efficient sequence-specific mRNA degradation in lysates and in cell culture. Different concentrations of siRNAs are used. An efficient concentration for suppression in vitro in mammalian culture is between 25 nM to 100 nM final concentration. This indicates that siRNAs are effective at concentrations that are several orders of magnitude below the concentrations applied in conventional antisense or ribozyme gene targeting experiments.

The above method provides a way both for the deduction of FGF-CX siRNA sequence and the use of such siRNA for in vitro suppression. In vivo suppression may be performed using the same siRNA using well known in vivo transfection or gene therapy transfection techniques.

Antisense Nucleic Acids

Another aspect of the invention pertains to isolated antisense nucleic acid molecules that are hybridizable to or complementary to the nucleic acid molecule comprising the nucleotide sequence of SEQ ID NOs: 1, 3, 5, 7, 9, 11, 13, 15, 17, 19, 21, 23, 25, 27, 29, 31, 33, and 35, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire FGF-CX coding strand, or to only a portion thereof. Nucleic acid molecules encoding fragments, homologs, derivatives and analogs of an FGF-CX protein of SEQ ID NOs: 2, 4, 6, 8, 10, 12, 14, 16, 18, 20, 22, 24, 26, 28, 30, 32, 34, 36, or antisense nucleic acids complementary to an FGF-CX nucleic acid sequence of SEQ ID NOs: 1, 3, 5, 7, 9, 11, 13, 15, 17, 19, 21, 23, 25, 27, 29, 31, 33, and 35, are additionally provided.

In one embodiment, an antisense nucleic acid molecule is antisense to a “coding region” of the coding strand of a nucleotide sequence encoding an FGF-CX protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding the FGF-CX protein. The term “noncoding region” refers to 5′ and 3′ sequences which flank the coding region that are not translated into amino acids (i.e., also referred to as 5′ and 3′ untranslated regions).

Given the coding strand sequences encoding the FGF-CX protein disclosed herein, antisense nucleic acids of the invention can be designed according to the rules of Watson and Crick or Hoogsteen base pairing. The antisense nucleic acid molecule can be complementary to the entire coding region of FGF-CX mRNA, but more preferably is an oligonucleotide that is antisense to only a portion of the coding or noncoding region of FGF-CX mRNA. For example, the antisense oligonucleotide can be complementary to the region surrounding the translation start site of FGF-CX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 5-methyluracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, uracil-5-oxyacetic acid methylester, uracil-5-oxyacetic acid (v), 5-methyl-2-thiouracil, 2,6-diaminopurine, (acp3)w, and 3-(3-amino-3-N-2-carboxypropyl) uracil. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding an FGF-CX protein to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An ct-anomeric nucleic acid molecule forms specific double-stranded hybrids with complementary RNA in which, contrary to the usual β-units, the strands run parallel to each other. See, e.g., Gaultier, et al., 1987. Nucl. Acids Res. 15: 6625-6641. The antisense nucleic acid molecule can also comprise a 2′-o-methylribonucleotide (see, e.g., Inoue, et al. 1987. Nucl. Acids Res. 15: 6131-6148) or a chimeric RNA-DNA analogue (see, e.g., Inoue, et al., 1987. FEBS Lett. 215: 327-330.

Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach 1988. Nature 334: 585-591) can be used to catalytically cleave FGF-CX mRNA transcripts to thereby inhibit translation of FGF-CX mRNA. A ribozyme having specificity for an FGF-CX-encoding nucleic acid can be designed based upon the nucleotide sequence of an FGF-CX cDNA disclosed herein (i.e., SEQ ID NOs: 1, 3, 5, 7, 9, 11, 13, 15, 17, 19, 21, 23, 25, 27, 29, 31, 33, and 35). For example, a derivative of a Tetrahymena L-19 IVS RNA can be constructed in which the nucleotide sequence of the active site is complementary to the nucleotide sequence to be cleaved in an FGF-CX-encoding mRNA. See, e.g., U.S. Pat. No. 4,987,071 to Cech, et al. and U.S. Pat. No. 5,116,742 to Cech, et al. FGF-CX mRNA can also be used to select a catalytic RNA having a specific ribonuclease activity from a pool of RNA molecules. See, e.g., Bartel et al., (1993) Science 261:1411-1418.

Alternatively, FGF-CX gene expression can be inhibited by targeting nucleotide sequences complementary to the regulatory region of the FGF-CX nucleic acid (e.g., the FGF-CX promoter and/or enhancers) to form triple helical structures that prevent transcription of the FGF-CX gene in target cells. See, e.g., Helene, 1991. Anticancer Drug Des. 6: 569-84; Helene, et al. 1992. Ann. N.Y. Acad. Sci. 660: 27-36; Maher, 1992. Bioassays 14: 807-15.

In various embodiments, the FGF-CX nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et al., 1996. Bioorg Med Chem 4: 5-23. As used herein, the terms “peptide nucleic acids” or “PNAs” refer to nucleic acid mimics (e.g., DNA mimics) in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup, et al., 1996. supra; Perry-O'Keefe, et al., 1996. Proc. Natl. Acad. Sci. USA 93: 14670-14675.

PNAs of FGF-CX can be used in therapeutic and diagnostic applications. For example, PNAs can be used as antisense or antigene agents for sequence-specific modulation of gene expression by, e.g., inducing transcription or translation arrest or inhibiting replication. PNAs of FGF-CX can also be used, for example, in the analysis of single base pair mutations in a gene (e.g., PNA directed PCR clamping; as artificial restriction enzymes when used in combination with other enzymes, e.g., S₁ nucleases (see, Hyrup, et al., 1996.supra); or as probes or primers for DNA sequence and hybridization (see, Hyrup, et al., 1996, supra; Perry-O'Keefe, et al., 1996. supra).

In another embodiment, PNAs of FGF-CX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of FGF-CX can be generated that may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleobases, and orientation (see, Hyrup, et al., 1996. supra). The synthesis of PNA-DNA chimeras can be performed as described in Hyrup, et al., 1996. supra and Finn, et al., 1996. Nucl Acids Res 24: 3357-3363. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA. See, e.g., Mag, et al., 1989. Nucl Acid Res 17: 5973-5988. PNA monomers are then coupled in a stepwise manner to produce a chimeric molecule with a 5′ PNA segment and a 3′ DNA segment. See, e.g., Finn, et al., 1996. supra. Alternatively, chimeric molecules can be synthesized with a 5′ DNA segment and a 3′ PNA segment. See, e.g., Petersen, et al., 1975. Bioorg. Med. Chem. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. WO88/09810) or the blood-brain barrier (see, e.g., PCT Publication No. WO 89/10134). In addition, oligonucleotides can be modified with hybridization triggered cleavage agents (see, e.g., Krol, et al., 1988. BioTechniques 6:958-976) or intercalating agents (see, e.g., Zon, 1988. Pharmi. Res. 5: 539-549). To this end, the oligonucleotide may be conjugated to another molecule, e.g., a peptide, a hybridization triggered cross-linking agent, a transport agent, a hybridization-triggered cleavage agent, and the like.

FGF-CX Polypeptides

A polypeptide according to the invention includes a polypeptide including the amino acid sequence of FGF-CX polypeptides whose sequences are provided in SEQ ID NOs: 2, 4, 6, 8, 10, 12, 14, 16, 18, 20, 22, 24, 26, 28, 30, 32, 34, 36. The invention also includes a mutant or variant protein any of whose residues may be changed from the corresponding residues shown in SEQ ID NOs: 2, 4, 6, 8, 10, 12, 14, 16, 18, 20, 22, 24, 26, 28, 30, 32, 34, 36, while still encoding a protein that maintains its FGF-CX activities and physiological functions, or a functional fragment thereof.

In general, an FGF-CX variant that preserves FGF-CX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated FGF-CX proteins, and biologically-active portions thereof, or derivatives, fragments, analogs or homologs thereof. Also provided are polypeptide fragments suitable for use as immunogens to raise anti-FGF-CX antibodies. In one embodiment, native FGF-CX proteins can be isolated from cells or tissue sources by an appropriate purification scheme using standard protein purification techniques. In another embodiment, FGF-CX proteins are produced by recombinant DNA techniques. Alternative to recombinant expression, an FGF-CX protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the FGF-CX protein is derived, or substantially free from chemical precursors or other chemicals when chemically synthesized. The language “substantially free of cellular material” includes preparations of FGF-CX proteins in which the protein is separated from cellular components of the cells from which it is isolated or recombinantly-produced. In one embodiment, the language “substantially free of cellular material” includes preparations of FGF-CX proteins having less than about 30% (by dry weight) of non-FGF-CX proteins (also referred to herein as a “contaminating protein”), more preferably less than about 20% of non-FGF-CX proteins, still more preferably less than about 10% of non-FGF-CX proteins, and most preferably less than about 5% of non-FGF-CX proteins. When the FGF-CX protein or biologically-active portion thereof is recombinantly-produced, it is also preferably substantially free of culture medium, i.e., culture medium represents less than about 20%, more preferably less than about 10%, and most preferably less than about 5% of the volume of the FGF-CX protein preparation.

The language “substantially free of chemical precursors or other chemicals” includes preparations of FGF-CX proteins in which the protein is separated from chemical precursors or other chemicals that are involved in the synthesis of the protein. In one embodiment, the language “substantially free of chemical precursors or other chemicals” includes preparations of FGF-CX proteins having less than about 30% (by dry weight) of chemical precursors or non-FGF-CX chemicals, more preferably less than about 20% chemical precursors or non-FGF-CX chemicals, still more preferably less than about 10% chemical precursors or non-FGF-CX chemicals, and most preferably less than about 5% chemical precursors or non-FGF-CX chemicals.

Biologically-active portions of FGF-CX proteins include peptides comprising amino acid sequences sufficiently homologous to or derived from the amino acid sequences of the FGF-CX proteins (e.g., the amino acid sequence shown in SEQ ID NOs: 2, 4, 6, 8, 10, 12, 14, 16, 18, 20, 22, 24, 26, 28, 30, 32, 34, 36) that include fewer amino acids than the full-length FGF-CX, and exhibit at least one activity of an FGF-CX protein. Typically, biologically-active portions comprise a domain or motif with at least one activity of the FGF-CX protein. A biologically-active portion of an FGF-CX protein can be a polypeptide which is, for example, 10, 25, 50, 100 or more amino acid residues in length.

Moreover, other biologically-active portions, in which other regions of the protein are deleted, can be prepared by recombinant techniques and evaluated for one or more of the functional activities of a native FGF-CX protein.

In an embodiment, the FGF-CX protein has an amino acid sequence shown in SEQ ID NOs: 2, 4, 6, 8, 10, 12, 14, 16, 18, 20, 22, 24, 26, 28, 30, 32, 34, 36. In other embodiments, the FGF-CX protein is substantially homologous to SEQ ID NOs: 2, 4, 6, 8, 10, 12, 14, 16, 18, 20, 22, 24, 26, 28, 30, 32, 34, 36, and retains the functional activity of the protein of SEQ ID NOs: 2, 4, 6, 8, 10, 12, 14, 16, 18, 20, 22, 24, 26, 28, 30, 32, 34, 36, yet differs in amino acid sequence due to natural allelic variation or mutagenesis, as described in detail, below. Accordingly, in another embodiment, the FGF-CX protein is a protein that comprises an amino acid sequence at least about 45% homologous to the amino acid sequence SEQ ID NOs: 2, 4, 6, 8, 10, 12, 14, 16, 18, 20, 22, 24, 26, 28, 30, 32, 34, 36, and retains the functional activity of the FGF-CX proteins of SEQ ID NOs: 2, 4, 6, 8, 10, 12, 14, 16, 18, 20, 22, 24, 26, 28, 30, 32, 34, 36.

Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence shown in SEQ ID NOs: 1, 3, 5, 7, 9, 11, 13, 15, 17, 19, 21, 23, 25, 27, 29, 31, 33, and 35.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides FGF-CX chimeric or fusion proteins. As used herein, an FGF-CX “chimeric protein” or “fusion protein” comprises an FGF-CX polypeptide operatively-linked to a non-FGF-CX polypeptide. An “FGF-CX polypeptide” refers to a polypeptide having an amino acid sequence corresponding to an FGF-CX protein (SEQ ID NOs: 2, 4, 6, 8, 10, 12, 14, 16, 18, 20, 22, 24, 26, 28, 30, 32, 34, 36), whereas a “non-FGF-CX polypeptide” refers to a polypeptide having an amino acid sequence corresponding to a protein that is not substantially homologous to the FGF-CX protein, e.g., a protein that is different from the FGF-CX protein and that is derived from the same or a different organism. Within an FGF-CX fusion protein the FGF-CX polypeptide can correspond to all or a portion of an FGF-CX protein. In one embodiment, an FGF-CX fusion protein comprises at least one biologically-active portion of an FGF-CX protein. In another embodiment, an FGF-CX fusion protein comprises at least two biologically-active portions of an FGF-CX protein. In yet another embodiment, an FGF-CX fusion protein comprises at least three biologically-active portions of an FGF-CX protein. Within the fusion protein, the term “operatively-linked” is intended to indicate that the FGF-CX polypeptide and the non-FGF-CX polypeptide are fused in-frame with one another. The non-FGF-CX polypeptide can be fused to the N-terminus or C-terminus of the FGF-CX polypeptide.

In one embodiment, the fusion protein is a GST-FGF-CX fusion protein in which the FGF-CX sequences are fused to the C-terminus of the GST (glutathione S-transferase) sequences. Such fusion proteins can facilitate the purification of recombinant FGF-CX polypeptides.

In another embodiment, the fusion protein is an FGF-CX protein containing a heterologous signal sequence at its N-terminus. In certain host cells (e.g., mammalian host cells), expression and/or secretion of FGF-CX can be increased through use of a heterologous signal sequence.

In yet another embodiment, the fusion protein is an FGF-CX-immunoglobulin fusion protein in which the FGF-CX sequences are fused to sequences derived from a member of the immunoglobulin protein family. The FGF-CX-immunoglobulin fusion proteins of the invention can be incorporated into pharmaceutical compositions and administered to a subject to inhibit an interaction between an FGF-CX ligand and an FGF-CX protein on the surface of a cell, to thereby suppress FGF-CX-mediated signal transduction in vivo. The FGF-CX-immunoglobulin fusion proteins can be used to affect the bioavailability of an FGF-CX cognate ligand. Inhibition of the FGF-CX ligand/FGF-CX interaction may be useful therapeutically for both the treatment of proliferative and differentiative disorders, as well as modulating (e.g. promoting or inhibiting) cell survival. Moreover, the FGF-CX-immunoglobulin fusion proteins of the invention can be used as immunogens to produce anti-FGF-CX antibodies in a subject, to purify FGF-CX ligands, and in screening assays to identify molecules that inhibit the interaction of FGF-CX with an FGF-CX ligand.

An FGF-CX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). An FGF-CX-encoding nucleic acid can be cloned into such an expression vector such that the fusion moiety is linked in-frame to the FGF-CX protein.

FGF-CX Agonists and Antagonists

The invention also pertains to variants of the FGF-CX proteins that function as either FGF-CX agonists (i.e., mimetics) or as FGF-CX antagonists. Variants of the FGF-CX protein can be generated by mutagenesis (e.g., discrete point mutation or truncation of the FGF-CX protein). An agonist of the FGF-CX protein can retain substantially the same, or a subset of, the biological activities of the naturally occurring form of the FGF-CX protein. An antagonist of the FGF-CX protein can inhibit one or more of the activities of the naturally occurring form of the FGF-CX protein by, for example, competitively binding to a downstream or upstream member of a cellular signaling cascade which includes the FGF-CX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the FGF-CX proteins.

Variants of the FGF-CX proteins that function as either FGF-CX agonists (i.e., mimetics) or as FGF-CX antagonists can be identified by screening combinatorial libraries of mutants (e.g., truncation mutants) of the FGF-CX proteins for FGF-CX protein agonist or antagonist activity. In one embodiment, a variegated library of FGF-CX variants is generated by combinatorial mutagenesis at the nucleic acid level and is encoded by a variegated gene library. A variegated library of FGF-CX variants can be produced by, for example, enzymatically ligating a mixture of synthetic oligonucleotides into gene sequences such that a degenerate set of potential FGF-CX sequences is expressible as individual polypeptides, or alternatively, as a set of larger fusion proteins (e.g., for phage display) containing the set of FGF-CX sequences therein. There are a variety of methods which can be used to produce libraries of potential FGF-CX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential FGF-CX sequences. Methods for synthesizing degenerate oligonucleotides are well-known within the art. See, e.g., Narang, 1983. Tetrahedron 39: 3; Itakura, et al., 1984. Annu. Rev. Biochem. 53: 323; Itakura, et al., 1984. Science 198: 1056; Ike, et al., 1983. Nucl. Acids Res. 11: 477.

Polypeptide Libraries

In addition, libraries of fragments of the FGF-CX protein coding sequences can be used to generate a variegated population of FGF-CX fragments for screening and subsequent selection of variants of an FGF-CX protein. In one embodiment, a library of coding sequence fragments can be generated by treating a double stranded PCR fragment of an FGF-CX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with S₁ nuclease, and ligating the resulting fragment library into an expression vector. By this method, expression libraries can be derived which encodes N-terminal and internal fragments of various sizes of the FGF-CX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of FGF-CX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify FGF-CX variants. See, e.g., Arkin and Yourvan, 1992. Proc. Natl. Acad. Sci. USA 89: 7811-7815; Delgrave, et al., 1993. Protein Engineering 6:327-331.

Anti-FGF-CX Antibodies

Also included in the invention are antibodies to FGF-CX proteins, or fragments of FGF-CX proteins. The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, F_{ab}, F_{ab′} and F_{(ab′)2} fragments, and an F_{ab} expression library. In general, an antibody molecule obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An isolated FGF-CX-related protein of the invention may be intended to serve as an antigen, or a portion or fragment thereof, and additionally can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of FGF-CX-related protein that is located on the surface of the protein, e.g., a hydrophilic region. A hydrophobicity analysis of the human FGF-CX-related protein sequence will indicate which regions of a FGF-CX-related protein are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each of which is incorporated herein by reference in its entirety. Antibodies that are specific for one or more domains within an antigenic protein, or derivatives, fragments, analogs or homologs thereof, are also provided herein.

A protein of the invention, or a derivative, fragment, analog, homolog or ortholog thereof, may be utilized as an immunogen in the generation of antibodies that immunospecifically bind these protein components.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow and Lane, 1988, Cold Spring Harbor Laboratory Press, Cold Spring Harbor, N.Y., incorporated herein by reference). Some of these antibodies are discussed below.

Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, MONOCLONAL ANTIBODIES: PRINCIPLES AND PRACTICE, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol., 133:3001 (1984); Brodeur et al., MONOCLONAL ANTIBODY PRODUCTION TECHNIQUES AND APPLICATIONS, Marcel Dekker, Inc., New York, (1987) pp. 51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). Preferably, antibodies having a high degree of specificity and a high binding affinity for the target antigen are isolated.

After the desired hybridoma cells are identified, the clones can be subcloned by limiting dilution procedures and grown by standard methods. Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539.) In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988; and Presta, Curr. Op. Struct. Biol., 2:593-596 (1992)).

Human Antibodies

Fully human antibodies relate to antibody molecules in which essentially the entire sequences of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonberg et al. (Nature 368 856-859 (1994)); Morrison (Nature 368, 812-13 (1994)); Fishwild et al, (Nature Biotechnology 14, 845-51 (1996)); Neuberger (Nature Biotechnology 14, 826 (1996)); and Lonberg and Huszar (Intern. Rev. Immunol. 13 65-93 (1995)).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

F_{ab} Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of F_{ab} expression libraries (see e.g., Huse, et al., 1989 Science 246: 1275-1281) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et al., 1991 EMBO J., 10:3655-3659.

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

Effector Function Engineering

It can be desirable to modify the antibody of the invention with respect to effector function, so as to enhance, e.g., the effectiveness of the antibody in treating cancer. For example, cysteine residue(s) can be introduced into the Fe region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-tumor activity can also be prepared using heterobifunctional cross-linkers as described in Wolff et al. Cancer Research, 53: 2560-2565 (1993). Alternatively, an antibody can be engineered that has dual Fe regions and can thereby have enhanced complement lysis and ADCC capabilities. See Stevenson et al., Anti-Cancer Drug Design, 3: 219-230 (1989).

Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

In one embodiment, methods for the screening of antibodies that possess the desired specificity include, but are not limited to, enzyme-linked immunosorbent assay (ELISA) and other immunologically-mediated techniques known within the art. In a specific embodiment, selection of antibodies that are specific to a particular domain of an FGF-CX protein is facilitated by generation of hybridomas that bind to the fragment of an FGF-CX protein possessing such a domain. Thus, antibodies that are specific for a desired domain within an FGF-CX protein, or derivatives, fragments, analogs or homologs thereof, are also provided herein.

Anti-FGF-CX antibodies may be used in methods known within the art relating to the localization and/or quantitation of an FGF-CX protein (e.g., for use in measuring levels of the FGF-CX protein within appropriate physiological samples, for use in diagnostic methods, for use in imaging the protein, and the like). In a given embodiment, antibodies for FGF-CX proteins, or derivatives, fragments, analogs or homologs thereof, that contain the antibody derived binding domain, are utilized as pharmacologically-active compounds (hereinafter “Therapeutics”).

An anti-FGF-CX antibody (e.g., monoclonal antibody) can be used to isolate an FGF-CX polypeptide by standard techniques, such as affinity chromatography or immunoprecipitation. An anti-FGF-CX antibody can facilitate the purification of natural FGF-CX polypeptide from cells and of recombinantly-produced FGF-CX polypeptide expressed in host cells. Moreover, an anti-FGF-CX antibody can be used to detect FGF-CX protein (e.g., in a cellular lysate or cell supernatant) in order to evaluate the abundance and pattern of expression of the FGF-CX protein. Anti-FGF-CX antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

FGF-CX Recombinant Expression Vectors and Host Cells

Another aspect of the invention pertains to vectors, preferably expression vectors, containing a nucleic acid encoding an FGF-CX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., FGF-CX proteins, mutant forms of FGF-CX proteins, fusion proteins, etc.).

The recombinant expression vectors of the invention can be designed for expression of FGF-CX proteins in prokaryotic or eukaryotic cells. For example, FGF-CX proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the FGF-CX expression vector is a yeast expression vector. Examples of vectors for expression in yeast Saccharomyces cerivisae include pYepSec1 (Baldari, et al., 1987. EMBO J. 6: 229-234), pMFa (Kudjan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, FGF-CX can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No.264, 166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to FGF-CX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein. A host cell can be any prokaryotic or eukaryotic cell. For example, FGF-CX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding FGF-CX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) FGF-CX protein. Accordingly, the invention further provides methods for producing FGF-CX protein using the host cells of the invention. In one embodiment, the method comprises culturing the host cell of invention (into which a recombinant expression vector encoding FGF-CX protein has been introduced) in a suitable medium such that FGF-CX protein is produced. In another embodiment, the method further comprises isolating FGF-CX protein from the medium or the host cell.

Transgenic FGF-CX Animals

The host cells of the invention can also be used to produce non-human transgenic animals. For example, in one embodiment, a host cell of the invention is a fertilized oocyte or an embryonic stem cell into which FGF-CX protein-coding sequences have been introduced. Such host cells can then be used to create non-human transgenic animals in which exogenous FGF-CX sequences have been introduced into their genome or homologous recombinant animals in which endogenous FGF-CX sequences have been altered. Such animals are useful for studying the function and/or activity of FGF-CX protein and for identifying and/or evaluating modulators of FGF-CX protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous FGF-CX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing FGF-CX-encoding nucleic acid into the male pronuclei of a fertilized oocyte (e.g., by microinjection, retroviral infection) and allowing the oocyte to develop in a pseudopregnant female foster animal. The human FGF-CX cDNA sequences of SEQ ID NOs: 1, 3, 5, 7, 9, 11, 13, 15, 17, 19, 21, 23, 25, 27, 29, 31, 33, and 35, can be introduced as a transgene into the genome of a non-human animal. Alternatively, a non-human homologue of the human FGF-CX gene, such as a mouse FGF-CX gene, can be isolated based on hybridization to the human FGF-CX cDNA (described further supra) and used as a transgene. Intronic sequences and polyadenylation signals can also be included in the transgene to increase the efficiency of expression of the transgene. A tissue-specific regulatory sequence(s) can be operably-linked to the FGF-CX transgene to direct expression of FGF-CX protein to particular cells. Methods for generating transgenic animals via embryo manipulation and microinjection, particularly animals such as mice, have become conventional in the art and are described, for example, in U.S. Pat. Nos. 4,736,866; 4,870,009; and 4, 873, 191; and Hogan, 1986. In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the FGF-CX transgene in its genome and/or expression of FGF-CX mRNA in tissues or cells of the animals. A transgenic founder animal can then be used to breed additional animals carrying the transgene. Moreover, transgenic animals carrying a transgene-encoding FGF-CX protein can further be bred to other transgenic animals carrying other transgenes.

To create a homologous recombinant animal, a vector is prepared which contains at least a portion of an FGF-CX gene into which a deletion, addition or substitution has been introduced to thereby alter, e.g., functionally disrupt, the FGF-CX gene. The FGF-CX gene can be a human gene (e.g., the cDNA of SEQ ID NOs: 1, 3, 5, 7, 9, 11, 13, 15, 17, 19, 21, 23, 25, 27, 29, 31, 33, and 35), but more preferably, is a non-human homologue of a human FGF-CX gene. For example, a mouse homologue of human FGF-CX gene of SEQ ID NOs: 1, 3, 5, 7, 9, 11, 13, 15, 17, 19, 21, 23, 25, 27, 29, 31, 33, and 35can be used to construct a homologous recombination vector suitable for altering an endogenous FGF-CX gene in the mouse genome. In one embodiment, the vector is designed such that, upon homologous recombination, the endogenous FGF-CX gene is functionally disrupted (i.e., no longer encodes a functional protein; also referred to as a “knock out” vector).

Alternatively, the vector can be designed such that, upon homologous recombination, the endogenous FGF-CX gene is mutated or otherwise altered but still encodes functional protein (e.g., the upstream regulatory region can be altered to thereby alter the expression of the endogenous FGF-CX protein). In the homologous recombination vector, the altered portion of the FGF-CX gene is flanked at its 5′- and 3′-termini by additional nucleic acid of the FGF-CX gene to allow for homologous recombination to occur between the exogenous FGF-CX gene carried by the vector and an endogenous FGF-CX gene in an embryonic stem cell. The additional flanking FGF-CX nucleic acid is of sufficient length for successful homologous recombination with the endogenous gene. Typically, several kilobases of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced FGF-CX gene has homologously-recombined with the endogenous FGF-CX gene are selected. See, e.g., Li, et al., 1992. Cell 69: 915.

The selected cells are then injected into a blastocyst of an animal (e.g., a mouse) to form aggregation chimeras. See, e.g., Bradley, 1987. In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, See, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter Go phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

Pharmaceutical Compositions

The FGF-CX nucleic acid molecules, FGF-CX proteins, and anti-FGF-CX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal (e.g., by mouthwash), and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., an FGF-CX protein or anti-FGF-CX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Screening and Detection Methods

The isolated nucleic acid molecules of the invention can be used to express FGF-CX protein (e.g., via a recombinant expression vector in a host cell in gene therapy applications), to detect FGF-CX mRNA (e.g., in a biological sample) or a genetic lesion in an FGF-CX gene, and to modulate FGF-CX activity, as described further, below. In addition, the FGF-CX proteins can be used to screen drugs or compounds that modulate the FGF-CX protein activity or expression as well as to treat disorders characterized by insufficient or excessive production of FGF-CX protein or production of FGF-CX protein forms that have decreased or aberrant activity compared to FGF-CX wild-type protein (e.g.; diabetes (regulates insulin release); obesity (binds and transport lipids); metabolic disturbances associated with obesity, the metabolic syndrome X as well as anorexia and wasting disorders associated with chronic diseases and various cancers, and infectious disease(possesses anti-microbial activity) and the various dyslipidemias. In addition, the anti-FGF-CX antibodies of the invention can be used to detect and isolate FGF-CX proteins and modulate FGF-CX activity. In yet a further aspect, the invention can be used in methods to influence appetite, absorption of nutrients and the disposition of metabolic substrates in both a positive and negative fashion.

The invention further pertains to novel agents identified by the screening assays described herein and uses thereof for treatments as described, supra.

Screening Assays

The invention provides a method (also referred to herein as a “screening assay”) for identifying modulators, i.e., candidate or test compounds or agents (e.g., peptides, peptidomimetics, small molecules or other drugs) that bind to FGF-CX proteins or have a stimulatory or inhibitory effect on, e.g., FGF-CX protein expression or FGF-CX a protein activity. The invention also includes compounds identified in the screening assays described herein.

In one embodiment, the invention provides assays for screening candidate or test compounds which bind to or modulate the activity of the membrane-bound form of an FGF-CX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37: 1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of FGF-CX protein, or a biologically-active portion thereof, on the cell surface is contacted with a test compound and the ability of the test compound to bind to an FGF-CX protein determined. The cell, for example, can of mammalian origin or a yeast cell. Determining the ability of the test compound to bind to the FGF-CX protein can be accomplished, for example, by coupling the test compound with a radioisotope or enzymatic label such that binding of the test compound to the FGF-CX protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of FGF-CX protein, or a biologically-active portion thereof, on the cell surface with a known compound which binds FGF-CX to form an assay mixture, contacting the assay mixture with a test compound, and determining the ability of the test compound to interact with an FGF-CX protein, wherein determining the ability of the test compound to interact with an FGF-CX protein comprises determining the ability of the test compound to preferentially bind to FGF-CX protein or a biologically-active portion thereof as compared to the known compound.

In another embodiment, an assay is a cell-based assay comprising contacting a cell expressing a membrane-bound form of FGF-CX protein, or a biologically-active portion thereof, on the cell surface with a test compound and determining the ability of the test compound to modulate (e.g., stimulate or inhibit) the activity of the FGF-CX protein or biologically-active portion thereof. Determining the ability of the test compound to modulate the activity of FGF-CX or a biologically-active portion thereof can be accomplished, for example, by determining the ability of the FGF-CX protein to bind to or interact with an FGF-CX target molecule. As used herein, a “target molecule” is a molecule with which an FGF-CX protein binds or interacts in nature, for example, a molecule on the surface of a cell which expresses an FGF-CX interacting protein, a molecule on the surface of a second cell, a molecule in the extracellular milieu, a molecule associated with the internal surface of a cell membrane or a cytoplasmic molecule. An FGF-CX target molecule can be a non-FGF-CX molecule or an FGF-CX protein or polypeptide of the invention. In one embodiment, an FGF-CX target molecule is a component of a signal transduction pathway that facilitates transduction of an extracellular signal (e.g. a signal generated by binding of a compound to a membrane-bound FGF-CX molecule) through the cell membrane and into the cell. The target, for example, can be a second intercellular protein that has catalytic activity or a protein that facilitates the association of downstream signaling molecules with FGF-CX.

Determining the ability of the FGF-CX protein to bind to or interact with an FGF-CX target molecule can be accomplished by one of the methods described above for determining direct binding. In one embodiment, determining the ability of the FGF-CX protein to bind to or interact with an FGF-CX target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising an FGF-CX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting an FGF-CX protein or biologically-active portion thereof with a test compound and determining the ability of the test compound to bind to the FGF-CX protein or biologically-active portion thereof. Binding of the test compound to the FGF-CX protein can be determined either directly or indirectly as described above. In one such embodiment, the assay comprises contacting the FGF-CX protein or biologically-active portion thereof with a known compound which binds FGF-CX to form an assay mixture, contacting the assay mixture with a test compound, and determining the ability of the test compound to interact with an FGF-CX protein, wherein determining the ability of the test compound to interact with an FGF-CX protein comprises determining the ability of the test compound to preferentially bind to FGF-CX or biologically-active portion thereof as compared to the known compound.

In still another embodiment, an assay is a cell-free assay comprising contacting FGF-CX protein or biologically-active portion thereof with a test compound and determining the ability of the test compound to modulate (e.g. stimulate or inhibit) the activity of the FGF-CX protein or biologically-active portion thereof. Determining the ability of the test compound to modulate the activity of FGF-CX can be accomplished, for example, by determining the ability of the FGF-CX protein to bind to an FGF-CX target molecule by one of the methods described above for determining direct binding. In an alternative embodiment, determining the ability of the test compound to modulate the activity of FGF-CX protein can be accomplished by determining the ability of the FGF-CX protein further modulate an FGF-CX target molecule. For example, the catalytic/enzymatic activity of the target molecule on an appropriate substrate can be determined as described, supra.

In yet another embodiment, the cell-free assay comprises contacting the FGF-CX protein or biologically-active portion thereof with a known compound which binds FGF-CX protein to form an assay mixture, contacting the assay mixture with a test compound, and determining the ability of the test compound to interact with an FGF-CX protein, wherein determining the ability of the test compound to interact with an FGF-CX protein comprises determining the ability of the FGF-CX protein to preferentially bind to or modulate the activity of an FGF-CX target molecule.

The cell-free assays of the invention are amenable to use of both the soluble form or the membrane-bound form of FGF-CX protein. In the case of cell-free assays comprising the membrane-bound form of FGF-CX protein, it may be desirable to utilize a solubilizing agent such that the membrane-bound form of FGF-CX protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, N-dodecyl--N,N-dimethyl-3-ammonio-1-propane sulfonate, 3-(3-cholamidopropyl) dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either FGF-CX protein or its target molecule to facilitate separation of complexed from uncomplexed forms of one or both of the proteins, as well as to accommodate automation of the assay. Binding of a test compound to FGF-CX protein, or interaction of FGF-CX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-FGF-CX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or FGF-CX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of FGF-CX protein binding or activity determined using standard techniques.

Other techniques for immobilizing proteins on matrices can also be used in the screening assays of the invention. For example, either the FGF-CX protein or its target molecule can be immobilized utilizing conjugation of biotin and streptavidin. Biotinylated FGF-CX protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with FGF-CX protein or target molecules, but which do not interfere with binding of the FGF-CX protein to its target molecule, can be derivatized to the wells of the plate, and unbound target or FGF-CX protein trapped in the wells by antibody conjugation. Methods for detecting such complexes, in addition to those described above for the GST-immobilized complexes, include immunodetection of complexes using antibodies reactive with the FGF-CX protein or target molecule, as well as enzyme-linked assays that rely on detecting an enzymatic activity associated with the FGF-CX protein or target molecule.

In another embodiment, modulators of FGF-CX protein expression are identified in a method wherein a cell is contacted with a candidate compound and the expression of FGF-CX mRNA or protein in the cell is determined. The level of expression of FGF-CX mRNA or protein in the presence of the candidate compound is compared to the level of expression of FGF-CX mRNA or protein in the absence of the candidate compound. The candidate compound can then be identified as a modulator of FGF-CX mRNA or protein expression based upon this comparison. For example, when expression of FGF-CX mRNA or protein is greater (i.e., statistically significantly greater) in the presence of the candidate compound than in its absence, the candidate compound is identified as a stimulator of FGF-CX mRNA or protein expression. Alternatively, when expression of FGF-CX mRNA or protein is less (statistically significantly less) in the presence of the candidate compound than in its absence, the candidate compound is identified as an inhibitor of FGF-CX mRNA or protein expression. The level of FGF-CX mRNA or protein expression in the cells can be determined by methods described herein for detecting FGF-CX mRNA or protein.

In yet another aspect of the invention, the FGF-CX proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with FGF-CX (“FGF-CX-binding proteins” or “FGF-CX-bp”) and modulate FGF-CX activity. Such FGF-CX-binding proteins are also likely to be involved in the propagation of signals by the FGF-CX proteins as, for example, upstream or downstream elements of the FGF-CX pathway.

The two-hybrid system is based on the modular nature of most transcription factors, which consist of separable DNA-binding and activation domains. Briefly, the assay utilizes two different DNA constructs. In one construct, the gene that codes for FGF-CX is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an FGF-CX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with FGF-CX.

The invention further pertains to novel agents identified by the aforementioned screening assays and uses thereof for treatments as described herein.

Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

Chromosome Mapping

Once the sequence (or a portion of the sequence) of a gene has been isolated, this sequence can be used to map the location of the gene on a chromosome. This process is called chromosome mapping. Accordingly, portions or fragments of the FGF-CX sequences, SEQ ID NOs: 1, 3, 5, 7, 9, 11, 13, 15, 17, 19, 21, 23, 25, 27, 29, 31, 33, and 35, or fragments or derivatives thereof, can be used to map the location of the FGF-CX genes, respectively, on a chromosome. The mapping of the FGF-CX sequences to chromosomes is an important first step in correlating these sequences with genes associated with disease.

Briefly, FGF-CX genes can be mapped to chromosomes by preparing PCR primers (preferably 15-25 bp in length) from the FGF-CX sequences. Computer analysis of the FGF-CX, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the FGF-CX sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the FGF-CX sequences to design oligonucleotide primers, sub-localization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., HUMAN CHROMOSOMES: A MANUAL OF BASIC TECHNIQUES (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g., in McKusick, MENDELIAN INHERITANCE IN MAN, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Moreover, differences in the DNA sequences between individuals affected and unaffected with a disease associated with the FGF-CX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

The FGF-CX sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” described in U.S. Pat. No. 5,272,057).

Furthermore, the sequences of the invention can be used to provide an alternative technique that determines the actual base-by-base DNA sequence of selected portions of an individual's genome. Thus, the FGF-CX sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The FGF-CX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If predicted coding sequences, such as those in SEQ ID NOs: 1, 3, 5, 7, 9, 11, 13, 15, 17, 19, 21, 23, 25, 27, 29, 31, 33, and 35, are used, a more appropriate number of primers for positive individual identification would be 500-2,000.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining FGF-CX protein and/or nucleic acid expression as well as FGF-CX activity, in the context of a biological sample (e.g., blood, serum, cells, tissue) to thereby determine whether an individual is afflicted with a disease or disorder, or is at risk of developing a disorder, associated with aberrant FGF-CX expression or activity. The disorders include pathology such as inflammatory conditions in the gastrointestinal tract, including but not limited to inflammatory bowel disease such as ulcerative colitis and Crohn's disease, growth and proliferative diseases such as cancer, angiogenesis, atherosclerotic plaques, collagen formation, cartilage and bone formation, cardiovascular and fibrotic diseases and diabetic ulcers. In addition, FGF-CX nucleic acids and their encoded polypeptides will be therapeutically useful for the prevention of aneurysms and the acceleration of wound closure through gene therapy. Furthermore, FGF-CX nucleic acids and their encoded polypeptides can be utilized to stimulate cellular growth wound healing, neovascularization and tissue growth, and similar tissue regeneration needs. More specifically, a FGF-CX nucleic acid or polypeptide may be useful in treatment of anemia and leukopenia, intestinal tract sensitivity and baldness. Treatment of such conditions may be indicated, e.g., in patients having undergone radiation or chemotherapy, wherein treatment would minimize any hyperproliferative side effects.

The invention also provides for prognostic (or predictive) assays for determining whether an individual is at risk of developing a disorder associated with FGF-CX protein, nucleic acid expression or activity. For example, mutations in an FGF-CX gene can be assayed in a biological sample. Such assays can be used for prognostic or predictive purpose to thereby prophylactically treat an individual prior to the onset of a disorder characterized by or associated with FGF-CX protein, nucleic acid expression, or biological activity.

Another aspect of the invention provides methods for determining FGF-CX protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of FGF-CX in clinical trials.

These and other agents are described in further detail in the following sections.

Diagnostic Assays

An exemplary method for detecting the presence or absence of FGF-CX in a biological sample involves obtaining a biological sample from a test subject and contacting the biological sample with a compound or an agent capable of detecting FGF-CX protein or nucleic acid (e.g., mRNA, genomic DNA) that encodes FGF-CX protein such that the presence of FGF-CX is detected in the biological sample. An agent for detecting FGF-CX mRNA or genomic DNA is a labeled nucleic acid probe capable of hybridizing to FGF-CX mRNA or genomic DNA. The nucleic acid probe can be, for example, a full-length FGF-CX nucleic acid, such as the nucleic acid of SEQ ID NOs: 1, 3, 5, 7, 9, 11, 13, 15, 17, 19, 21, 23, 25, 27, 29, 31, 33, and 35, or a portion thereof, such as an oligonucleotide of at least 15, 30, 50, 100, 250 or 500 nucleotides in length and sufficient to specifically hybridize under stringent conditions to FGF-CX mRNA or genomic DNA. Other suitable probes for use in the diagnostic assays of the invention are described herein.

An agent for detecting FGF-CX protein is an antibody capable of binding to FGF-CX protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect FGF-CX mRNA, protein, or genomic DNA in a biological sample in vitro as well as in vivo. For example, in vitro techniques for detection of FGF-CX mRNA include Northern hybridizations and in situ hybridizations. In vitro techniques for detection of FGF-CX protein include enzyme linked immunosorbent assays (ELISAs), Western blots, immunoprecipitations, and immunofluorescence. In vitro techniques for detection of FGF-CX genomic DNA include Southern hybridizations. Furthermore, in vivo techniques for detection of FGF-CX protein include introducing into a subject a labeled anti-FGF-CX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting FGF-CX protein, mRNA, or genomic DNA, such that the presence of FGF-CX protein, mRNA or genomic DNA is detected in the biological sample, and comparing the presence of FGF-CX protein, mRNA or genomic DNA in the control sample with the presence of FGF-CX protein, mRNA or genomic DNA in the test sample.

The invention also encompasses kits for detecting the presence of FGF-CX in a biological sample. For example, the kit can comprise: a labeled compound or agent capable of detecting FGF-CX protein or mRNA in a biological sample; means for determining the amount of FGF-CX in the sample; and means for comparing the amount of FGF-CX in the sample with a standard. The compound or agent can be packaged in a suitable container. The kit can further comprise instructions for using the kit to detect FGF-CX protein or nucleic acid.

Prognostic Assays

The diagnostic methods described herein can furthermore be utilized to identify subjects having or at risk of developing a disease or disorder associated with aberrant FGF-CX expression or activity. For example, the assays described herein, such as the preceding diagnostic assays or the following assays, can be utilized to identify a subject having or at risk of developing a disorder associated with FGF-CX protein, nucleic acid expression or activity. Alternatively, the prognostic assays can be utilized to identify a subject having or at risk for developing a disease or disorder. Thus, the invention provides a method for identifying a disease or disorder associated with aberrant FGF-CX expression or activity in which a test sample is obtained from a subject and FGF-CX protein or nucleic acid (e.g., mRNA, genomic DNA) is detected, wherein the presence of FGF-CX protein or nucleic acid is diagnostic for a subject having or at risk of developing a disease or disorder associated with aberrant FGF-CX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant FGF-CX expression or activity. For example, such methods can be used to determine whether a subject can be effectively treated with an agent for a disorder. Thus, the invention provides methods for determining whether a subject can be effectively treated with an agent for a disorder associated with aberrant FGF-CX expression or activity in which a test sample is obtained and FGF-CX protein or nucleic acid is detected (e.g., wherein the presence of FGF-CX protein or nucleic acid is diagnostic for a subject that can be administered the agent to treat a disorder associated with aberrant FGF-CX expression or activity).

The methods of the invention can also be used to detect genetic lesions in an FGF-CX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding an FGF-CX-protein, or the misexpression of the FGF-CX gene. For example, such genetic lesions can be detected by ascertaining the existence of at least one of: (i) a deletion of one or more nucleotides from an FGF-CX gene; (ii) an addition of one or more nucleotides to an FGF-CX gene; (iii) a substitution of one or more nucleotides of an FGF-CX gene, (iv) a chromosomal rearrangement of an FGF-CX gene; (v) an alteration in the level of a messenger RNA transcript of an FGF-CX gene, (vi) aberrant modification of an FGF-CX gene, such as of the methylation pattern of the genomic DNA, (vii) the presence of a non-wild-type splicing pattern of a messenger RNA transcript of an FGF-CX gene, (viii) a non-wild-type level of an FGF-CX protein, (ix) allelic loss of an FGF-CX gene, and (x) inappropriate post-translational modification of an FGF-CX protein. As described herein, there are a large number of assay techniques known in the art which can be used for detecting lesions in an FGF-CX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4, 683, 202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the FGF-CX-gene (see, Abravaya, et al., 1995. Nuc. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to an FGF-CX gene under conditions such that hybridization and amplification of the FGF-CX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Natl. Acad. Sci. USA 86: 1173-1177); Qua Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in an FGF-CX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in FGF-CX can be identified by hybridizing a sample and control nucleic acids, e.g., DNA or RNA, to high-density arrays containing hundreds or thousands of oligonucleotides probes. See, e.g., Cronin, et al., 1996. Human Mutation 7: 244-255; Kozal, et al., 1996. Nat. Med. 2: 753-759. For example, genetic mutations in FGF-CX can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the FGF-CX gene and detect mutations by comparing the sequence of the sample FGF-CX with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the FGF-CX gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type FGF-CX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S₁ nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in FGF-CX cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on an FGF-CX sequence, e.g., a wild-type FGF-CX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in FGF-CX genes. For example, single strand conformation polymorphism (SSCP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control FGF-CX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell Probes 6: 1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving an FGF-CX gene.

Furthermore, any cell type or tissue, preferably peripheral blood leukocytes, in which FGF-CX is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on FGF-CX activity (e.g., FGF-CX gene expression), as identified by a screening assay described herein can be administered to individuals to treat (prophylactically or therapeutically) disorders. The disorders include pathology such as inflammatory conditions in the gastrointestinal tract, including but not limited to inflammatory bowel disease such as ulcerative colitis and Crohn's disease, growth and proliferative diseases such as cancer, angiogenesis, atherosclerotic plaques, collagen formation, cartilage and bone formation, cardiovascular and fibrotic diseases and diabetic ulcers. In addition, FGF-CX nucleic acids and their encoded polypeptides will be therapeutically useful for the prevention of aneurysms and the acceleration of wound closure through gene therapy. Furthermore, FGF-CX nucleic acids and their encoded polypeptides can be utilized to stimulate cellular growth wound healing, neovascularization and tissue growth, and similar tissue regeneration needs. More specifically, a FGF-CX nucleic acid or polypeptide may be useful in treatment of anemia and leukopenia, intestinal tract sensitivity and baldness. Treatment of such conditions may be indicated, e.g., in patients having undergone radiation or chemotherapy, wherein treatment would minimize any hyperproliferative side effects.

n conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of FGF-CX protein, expression of FGF-CX nucleic acid, or mutation content of FGF-CX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol., 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C 19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of FGF-CX protein, expression of FGF-CX nucleic acid, or mutation content of FGF-CX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with an FGF-CX modulator, such as a modulator identified by one of the exemplary screening assays described herein.

Monitoring of Effects During Clinical Trials

Monitoring the influence of agents (e.g., drugs, compounds) on the expression or activity of FGF-CX (e.g., the ability to modulate aberrant cell proliferation and/or differentiation) can be applied not only in basic drug screening, but also in clinical trials. For example, the effectiveness of an agent determined by a screening assay as described herein to increase FGF-CX gene expression, protein levels, or upregulate FGF-CX activity, can be monitored in clinical trails of subjects exhibiting decreased FGF-CX gene expression, protein levels, or downregulated FGF-CX activity. Alternatively, the effectiveness of an agent determined by a screening assay to decrease FGF-CX gene expression, protein levels, or downregulate FGF-CX activity, can be monitored in clinical trails of subjects exhibiting increased FGF-CX gene expression, protein levels, or upregulated FGF-CX activity. In such clinical trials, the expression or activity of FGF-CX and, preferably, other genes that have been implicated in, for example, a cellular proliferation or immune disorder can be used as a “read out” or markers of the immune responsiveness of a particular cell.

By way of example, and not of limitation, genes, including FGF-CX, that are modulated in cells by treatment with an agent (e.g., compound, drug or small molecule) that modulates FGF-CX activity (e.g., identified in a screening assay as described herein) can be identified. Thus, to study the effect of agents on cellular proliferation disorders, for example, in a clinical trial, cells can be isolated and RNA prepared and analyzed for the levels of expression of FGF-CX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of FGF-CX or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of an FGF-CX protein, mRNA, or genomic DNA in the preadministration sample; (iii) obtaining one or more post-administration samples from the subject; (iv) detecting the level of expression or activity of the FGF-CX protein, mRNA, or genomic DNA in the post-administration samples; (v) comparing the level of expression or activity of the FGF-CX protein, mRNA, or genomic DNA in the pre-administration sample with the FGF-CX protein, mRNA, or genomic DNA in the post administration sample or samples; and (vi) altering the administration of the agent to the subject accordingly. For example, increased administration of the agent may be desirable to increase the expression or activity of FGF-CX to higher levels than detected, i.e., to increase the effectiveness of the agent. Alternatively, decreased administration of the agent may be desirable to decrease expression or activity of FGF-CX to lower levels than detected, i.e., to decrease the effectiveness of the agent.

Methods of Treatment

The invention provides methods of treating, preventing or alleviating a symptom of a tissue proliferative disorder. The tissue proliferative disorder is acute or chronic. Tissue proliferative disorders include, for example, oral mucositis, oral candidiasis, tumors, restinosis, psoriasis, diabetic and post surgery complications, irritable bowel disease, rheumatoid arthritis, cancer, radiation sickness, ischemic stroke, hemorrhagic stroke, trauma, spinal cord damage, heavy metal or toxin poisoning, neurodegenerative diseases (such as Alzheimer's, Parkinson's Disease, Amyotrophic Lateral Sclerosis, Huntington's Disease), and osteoarthritis. The methods include identifying a subject suffering from or at risk of developing a tissue proliferative disorder and administering to a subject a protein, a nucleic acid or fragments thereof.

Tissue proliferative disorders are characterized by aberrant cell proliferation. (i.e., increase or decrease of proliferation) Cell proliferation is measured by methods known in the art, such as the MTT cell proliferation assay.

A subject suffering from or at risk of developing a tissue proliferative disorder is identified by methods known in the art. Tissue proliferation-associated disorders are diagnosed and or monitored, typically by a physician using standard methodologies. For example, mucositis progresses through three stages. In Stage 1, inflammation is accompanied by painful mucosal erythema, which can respond to local anesthetics. In Stage 2, there is painful ulceration with pseudomembrane formation and the pain is often of such intensity as to require parenteral narcotic analgesia. In the case of myelosuppressive treatment, there is also potentially life-threatening sepsis, requiring antimicrobial therapy. In Stage 3, there is spontaneous healing, occurring about 2-3 weeks after cessation of anti-neoplastic therapy. Standard therapy for mucositis is predominantly palliative, including application of topical analgesics such as lidocaine and/or systemic administration of narcotics and antibiotics.

Inhibition of the symptoms of a tissue proliferative disorder is characterized by a stimulation of DNA synthesis in cells of mesenchymal, epithelial or endothelial origin. DNA synthesis is measured by methods know in the art. For example, DNA synthesis is measured by BrdU incorporation.

Efficaciousness of treatment is determined in association with any known method for diagnosing or treating the particular tissue proliferation-associated disorder. Alleviation of one or more symptoms of the tissue proliferation-associated disorder indicates that the compound confers a clinical benefit.

The methods described herein lead to a reduction in the severity or the allevialtion of one or more symptoms of a tissue proliferation-associated disorder such as those described herein. Tissue proliferation-associated disorders are diagnosed and or monitored, typically by a physician using standard methodologies.

The invention provides for both prophylactic and therapeutic methods of treating a subject at risk of (or susceptible to) a disorder or having a disorder associated with aberrant FGF-CX expression or activity. The disorders include cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, adrenoleukodystrophy, congenital adrenal hyperplasia, prostate cancer, neoplasm; adenocarcinoma, lymphoma, uterus cancer, fertility, hemophilia, hypercoagulation, idiopathic thrombocytopenic purpura, immunodeficiencies, graft versus host disease, AIDS, bronchial asthma, Crohn's disease; multiple sclerosis, treatment of Albright Hereditary Ostoeodystrophy, and other diseases, disorders and conditions of the like.

These methods of treatment will be discussed more fully, below.

Disease and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endoggenous function of an aforementioned peptide by homologous recombination (see, e.g., Capecchi, 1989. Science 244: 1288-1292); or (v) modulators (i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

In one aspect, the invention provides a method for preventing, in a subject, a disease or condition associated with an aberrant FGF-CX expression or activity, by administering to the subject an agent that modulates FGF-CX expression or at least one FGF-CX activity. Subjects at risk for a disease that is caused or contributed to by aberrant FGF-CX expression or activity can be identified by, for example, any or a combination of diagnostic or prognostic assays as described herein. Administration of a prophylactic agent can occur prior to the manifestation of symptoms characteristic of the FGF-CX aberrancy, such that a disease or disorder is prevented or, alternatively, delayed in its progression. Depending upon the type of FGF-CX aberrancy, for example, an FGF-CX agonist or FGF-CX antagonist agent can be used for treating the subject. The appropriate agent can be determined based on screening assays described herein. The prophylactic methods of the invention are further discussed in the following subsections.

Therapeutic Methods

Another aspect of the invention pertains to methods of modulating FGF-CX expression or activity for therapeutic purposes. The modulatory method of the invention involves contacting a cell with an agent that modulates one or more of the activities of FGF-CX protein activity associated with the cell. An agent that modulates FGF-CX protein activity can be an agent as described herein, such as a nucleic acid or a protein, a naturally-occurring cognate ligand of an FGF-CX protein, a peptide, an FGF-CX peptidomimetic, or other small molecule. In one embodiment, the agent stimulates one or more FGF-CX protein activity. Examples of such stimulatory agents include active FGF-CX protein and a nucleic acid molecule encoding FGF-CX that has been introduced into the cell. In another embodiment, the agent inhibits one or more FGF-CX protein activity. Examples of such inhibitory agents include antisense FGF-CX nucleic acid molecules and anti-FGF-CX antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of an FGF-CX protein or nucleic acid molecule. In one embodiment, the method involves administering an agent (e.g., an agent identified by a screening assay described herein), or combination of agents that modulates (e.g., up-regulates or down-regulates) FGF-CX expression or activity. In another embodiment, the method involves administering an FGF-CX protein or nucleic acid molecule as therapy to compensate for reduced or aberrant FGF-CX expression or activity.

Stimulation of FGF-CX activity is desirable in situations in which FGF-CX is abnormally downregulated and/or in which increased FGF-CX activity is likely to have a beneficial effect. One example of such a situation is where a subject has a disorder characterized by aberrant cell proliferation and/or differentiation (e.g., cancer or immune associated disorders). Another example of such a situation is where the subject has a gestational disease (e.g., preclampsia).

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Prophylactic and Therapeutic Uses of the Compositions of the Invention

The FGF-CX nucleic acids and proteins of the invention are useful in potential prophylactic and therapeutic applications implicated in a variety of disorders including, but not limited to: inflammatory bowel disease and disorders associated with FGF-CX.

As an example, a cDNA encoding the FGF-CX protein of the invention may be useful in gene therapy, and the protein may be useful when administered to a subject in need thereof. By way of non-limiting example, the compositions of the invention will have efficacy for treatment of patients suffering from: oral mucositis, oral candidiasis, tumors, restinosis, psoriasis, diabetic and post surgery complications, rheumatoid arthritis, cancer, radiation sickness, ischemic stroke, hemorrhagic stroke, trauma, spinal cord damage, heavy metal or toxin poisoning, neurodegenerative diseases (such as Alzheimer's, Parkinson's Disease, Amyotrophic Lateral Sclerosis, Huntington's Disease), osteoarthritis, inflammatory conditions in the gastrointestinal tract, including but not limited to inflammatory bowel disease such as ulcerative colitis and Crohn's disease, growth and proliferative diseases such as cancer, angiogenesis, atherosclerotic plaques, collagen formation, cartilage and bone formation, cardiovascular and fibrotic diseases and diabetic ulcers.

The novel nucleic acid encoding the FGF-CX protein, or nucleic acid or protein fragments, analogs, homologs or derivative thereof, may also be useful in diagnostic applications, wherein the presence or amount of the nucleic acid or the protein are to be assessed. A further use could be as an anti-bacterial molecule (i.e., some peptides have been found to possess anti-bacterial properties). These materials are further useful in the generation of antibodies which immunospecifically-bind to the novel substances of the invention for use in therapeutic or diagnostic methods.

### EXAMPLES

### Example 1

Polynucleotide and Polypeptide Sequences, and Homology Data

Details of the sequence relatedness and domain analysis for each FGF-CX are presented in Table 1A. The FGF-CX1 clone was analyzed, and the nucleotide and encoded polypeptide sequences are shown in Table 1A.

TABLE 1A
FGF-CX1 Sequence Analysis

| | | |
|---|---|---|
| FGF-CX1a, CG53135-05 | SEQ ID NO:1 | 636 bp |
| DNA Sequence | ORF Start: ATG at 1 | ORF Stop: end of sequence |

ATGGCTCCGCTGGCTGAAGTTGGTGGTTTCCTGGGCGGTCTGGAGGGTCTGGGTCAGCAGGTTGGTTC
TCACTTCCTGCTGCCGCCGGCTGGTGAACGTCCGCCACTGCTGGGTGAACGTCGCTCCGCAGCTGAAC
GCTCCGCTCGTGGTGGCCCGGGTGCTGCTCAGCTGGCTCACCTGCATGGTATCCTGCGTCGCCGTCAG
CTGTACTGCCGTACTGGTTTCCACCTGCAGATCCTGCCGGATGGTTCTGTTCAGGGTACCCGTCAGGA
CCACTCTCTGTTCGGTATCCTGGAATTCATCTCTGTTGCTGTTGGTCTGGTTTCTATCCGTGGTGTTG
ACTCTGGCCTGTACCTGGGTATGAACGACAAAGGCGAACTGTACGGTTCTGAAAAACTGACCTCTGAA
TGCATCTTCCGTGAACAGTTTGAAGAGAACTGGTACAACACCTACTCTTCCAACATCTACAAACATGG
TGACACCGGCCGTCGCTACTTCGTTGCTCTGAACAAAGACGGTACCCCGCGTGATGGTGCTCGTTCTA
AACGTCACCAGAAATTCACCCACTTCCTGCCGCGCCCAGTTGACCCGGAGCGTGTTCCAGAACTGTAT
AAAGACCTGCTGATGTACACCTAA

| | | | |
|---|---|---|---|
| FGF-CX1a, CG53135-05 | SEQ ID NO: 2 | 211 aa | MW at 23498.4 kD |
| Protein Sequence | | | |

MAPLAEVGGFLGGLEGLGQQVGSHFLLPPAGERPPLLGERRSAAERSARGGPGAAQLAHLHGILRRRQ
LYCRTGFHLQILPDGSVQGTRQDHSLFGILEFISVAVGLVSIRGVDSGLYLGMNDKGELYGSEKLTSE
CIFREQFEENWYNTYSSNIYKHGDTGRRYFVALNKDGTPRDGARSKRHQKFTHFLPRPVDPERVPELY
KDLLMYT

| | | |
|---|---|---|
| FGF-CX1b, CG53135-01 | SEQ ID NO: 3 | 633 bp |
| DNA Sequence | ORF Start: ATG at 1 | ORF Stop: |

ATGGCTCCCTTAGCCGAAGTCGGGGGCTTTCTGGGCGGCCTGGAGGGCTTGGGCCAGCAGGTGGGTTC
GCATTTCCTGTTGCCTCCTGCCGGGGAGCGGCCGCCGCTGCTGGGCGAGCGCAGGAGCGCGGCGGAGC
GGAGCGCGCGCGGCGGGCCGGGGGCTGCGCAGCTGGCGCACCTGCACGGCATCCTGCGCCGCCGGCAG
CTCTATTGCCGCACCGGCTTCCACCTGCAGATCCTGCCCGACGGCAGCGTGCAGGGCACCCGGCAGGA
CCACAGCCTCTTCGGTATCTTGGAATTCATCAGTGTGGCAGTGGGACTGGTCAGTATTAGAGGTGTGG
ACAGTGGTCTCTATCTTGGAATGAATGACAAAGGAGAACTCTATGGATCAGAGAAACTTACTTCCGAA
TGCATCTTTAGGGAGCAGTTTGAAGAGAACTGGTATAACACCTATTCATCTAACATATATAAACATGG
AGACACTGGCCGCAGGTATTTTGTGGCACTTAACAAAGACGGAACTCCAAGAGATGGCGCCAGGTCCA
AGAGGCATCAGAAATTTACACATTTCTTACCTAGACCAGTGGATCCAGAAAGAGTTCCAGAATTGTAC
AAGGACCTACTGATGTACACT

| | | | |
|---|---|---|---|
| FGF-CX1b, CG53135-01 | SEQ ID NO:4 | 211 aa | MW at 23498.4kD |
| Protein Sequence | | | |

MAPLAEVGGFLGGLEGLGQQVGSHFLLPPAGERPPLLGERRSAAERSARGGPGAAQLAHLHGILRRRQ
LYCRTGFHLQILPDGSVQGTRQDHSLFGILEFISVAVGLVSIRGVDSGLYLGMNDKGELYGSEKLTSE
CIFREQFEENWYNTYSSNIYKHGDTGRRYFVALNKDGTPRDGARSKRHQKFTHFLPRPVDPERVPELY
KDLLMYT

| | | |
|---|---|---|
| FGF-CX1c, CG53135-04 | SEQ ID NO: 5 | 540 bp |
| DNA Sequence | ORF Start: ATG at 1 | ORF Stop: end of sequence |

ATGGCTCCCTTAGCCGAAGTCGGGGGCTTTCTGGGCGGCCTGGAGGGCTTGGGCCAGCCGGGGGCAGC
GCAGCTGGCGCACCTGCACGGCATCCTGCGCCGCCGGCAGCTCTATTGCCGCACCGGCTTCCACCTGC
AGATCCTGCCCGACGGCAGCGCGCAGGGCACCCGGCAGGACCACAGCCTCTTCGGTATCTTGGAATTC
ATCAGTGTGGCAGTGGGACTGGTCAGTATTAGAGGTGTGGACAGTGGTCTCTATCTTGGAATGAATGA
CAAAGGAGAACTCTATGGATCAGAGAAACTTACTTCCGAATGCATCTTTAGGGAGCAGTTTGAAGAGA
ACTGGTATAACACCTATTCATCTAACATATATAAACATGGAGACACTGGCCGCAGGTATTTTGTGGCA
CTTAACAAAGACGGAACTCCAAGAGATGGCGCCAGGTCCAAGAGGCATCAGAAATTTACACATTTCTT
ACCTAGACCAGTGGATCCAGAAAGAGTTCCAGAATTGTACAAGGACCTACTGATGTACACTTAG

| | | | |
|---|---|---|---|
| FGF-CX1c, CG53135-04 | SEQ ID NO: 6 | 179 aa | MW at 20118.6kD |
| Protein Sequence | | | |

MAPLAEVGGFLGGLEGLGQPGAAQLAHLHGILRRRQLYCRTGFHLQILPDGSAQGTRQDHSLFGILEF
ISVAVGLVSIRGVDSGLYLGMNDKGELYGSEKLTSECIFREQFEENWYNTYSSNIYKHGDTGRRYFVA
LNKDGTPRDGARSKRHQKFTHFLPRPVDPERVPELYKDLLMYT

| | | |
|---|---|---|
| FGF-CX1d, 250059596 | SEQ ID NO: 7 | 556 bp |
| DNA Sequence | ORF Start: | ORF Stop: |

CACCAGATCTATGGCTCCCTTAGCCGAAGTCGGGGGCTTTCTGGGCGGCCTGGAGGGCTTGGGCCAGC
CGGGGGCAGCGCAGCTGGCGCACCTGCACGGCATCCTGCGCCGCCGGCAGCTCTATTGCCGCACCGGC
TTCCACCTGCAGATCCTGCCCGACGGCAGCGTGCAGGGCACCCGGCAGGACCACAGCCTCTTCGGTAT
CTTGGAATTCATCAGTGTGGCAGTGGGACTGGTCAGTATTAGAGGTGTGGACAGTGGTCTCTATCTTG
GAATGAATGACAAAGGAGAACTCTATGGATCAGAGAAACTTACTTCCGAATGCATCTTTAGGGAGCAG
TTTGAAGAGAACTGGTATAACACCTATTCATCTAACATATATAAACATGGAGACACTGGCCGCAGGTA
TTTTGTGGCACTTAACAAAGACGGAACTCCAAGAGATGGCGCCAGGTCCAAGAGGCATCAGAAATTTA
CACATTTCTTACCTAGACCAGTGGATCCAGAAAGAGTTCCAGAATTGTACAAGGACCTACTGATGTAC
ACTGTCGACGGC

| | | | |
|---|---|---|---|
| FGF-CX1d, 250059596 | SEQ ID NO: 8 | 185 aa | MW at 20762.3kD |
| Protein Sequence | | | |

TRSMAPLAEVGGFLGGLEGLGQPGAAQLAHLHGILRRRQLYCRTGFHLQILPDGSVQGTRQDHSLFGI
LEFISVAVGLVSIRGVDSGLYLGMNDKGELYGSEKLTSECIFREQFEENWYNTYSSNIYKHGDTGRRY
FVALNKDGTPRDGARSKRHQKFTHFLPRPVDPERVPELYKDLLMYTVDG

| | | |
|---|---|---|
| FGF-CX1e, 250059629 | SEQ ID NO: 9 | 415 bp |
| DNA Sequence | ORF Start: | ORF Stop: |

CACCAGATCTATCCTGCGCCGCCGGCAGCTCTATTGCCGCACCGGCTTCCACCTGCAGATCCTGCCCG
ACGGCAGCGTGCAGGGCACCCGGCAGGACCACAGCCTCTTCGGTATCTTGGAATTCATCAGTGTGGCA
GTGGGACTGGTCAGTATTAGAGGTGTGGACAGTGGTCTCTATCTTGGAATGAATGACAAAGGAGAACT
CTATGGATCAGAGAAACTTACTTCCGAATGCATCTTTAGGGAGCAGTTTGAAGAGAACTGGTATAACA
CCTATTCATCTAACATATATAAACATGGAGACACTGGCCGCAGGTATTTTGTGGCACTTAACAAAGAC
GGAACTCCAAGAGATGGCGCCAGGTCCAAGAGGCATCAGAAATTTACACATTTCTTACCTAGACCAGT
CGACGGC

| | | | |
|---|---|---|---|
| FGF-CX1e, 250059629 | SEQ ID NO: 10 | 138 aa | MW at 15847.7kD |
| Protein Sequence | | | |

TRSILRRRQLYCRTGFHLQILPDGSVQGTRQDHSLFGILEFISVAVGLVSIRGVDSGLYLGMNDKGEL
YGSEKLTSECIFREQFEENWYNTYSSNIYKHGDTGRRYFVALNKDGTPRDGARSKRHQKFTHFLPRPV
DG

| | | |
|---|---|---|
| FGF-CX1f, 250059669 | SEQ ID NO: 11 | 466 bp |
| DNA Sequence | ORF Start: | ORF Stop: |

CACCAGATCTATCCTGCGCCGCCGGCAGCTCTATTGCCGCACCGGCTTCCACCTGCAGATCCTGCCCG
ACGGCAGCGTGCAGGGCACCCGGCAGGACCACAGCCTCTTCGGTATCTTGGAATTCATCAGTGTGGCA
GTGGGACTGGTCAGTATTAGAGGTGTGGACAGTGGTCTCTATCTTGGAATGAATGACAAAGGAGAACT
CTATGGATCAGAGAAACTTACTTCCGAATGCATCTTTAGGGAGCAGTTTGAAGAGAACTGGTATAACA
CCTATTCATCTAACATATATAAACATGGAGACACTGGCCGCAGGTATTTTGTGGCACTTAACAAAGAC
GGAACTCCAAGAGATGGCGCCAGGTCCAAGAGGCATCAGAAATTTACACATTTCTTACCTAGACCAGT
GGATCCAGAAAGAGTTCCAGAATTGTACAAGGACCTACTGATGTACACTGTCGACGGC

| | | | |
|---|---|---|---|
| FGF-CX1f, 250059669 | SEQ ID NO: 12 | 155 aa | MW at 17911.1kD |
| Protein Sequence | | | |

TRSILRRRQLYCRTGFHLQILPDGSVQGTRQDHSLFGILEFISVAVGLVSIRGVDSGLYLGMNDKGEL
YGSEKLTSECIFREQFEENWYNTYSSNIYKHGDTGRRYFVALNKDGTPRDGARSKRHQKFTHFLPRPV
DPERVPELYKDLLMYTVDG

| | | |
|---|---|---|
| FGF-CX1g, 316351224 | SEQ ID NO: 13 | 549 bp |
| DNA Sequence | ORF: Start: | ORF Stop: |

AGATCTATGGCTCCCTTAGCCGAAGTCGGGGGCTTTCTGGGCGGCCTGGAGGGCTTGGGCCAGCCGGG
GGCAGCGCAGCTGGCGCACCTGCACGGCATCCTGCGCCGCCGGCAGCTCTATTGCCGCACCGGCTTCC
ACCTGCAGATCCTGCCCGACGGCAGCGTGCAGGGCACCCGGCAGGACCACAGCCTCTTCGGTATCTTG
GAATTCATCAGTGTGGCAGTGGGACTGGTCAGTATTAGAGGTGTGGACAGTGGTCTCTATCTTGGAAT
GAATGACAAAGGAGAACTCTATGGATCAGAGAAACTTACTTCCGAATGCATCTTTAGGGAGCAGTTTG
AAGAGAACTGGTATAACACCTATTCATCTAACATATATAAACATGGAGACACTGGCCGCAGGTATTTT
GTGGCACTTAACAAAGACGGAACTCCAAGAGATGGCGCCAGGTCCAAGAGGCATCAGAAATTTACACA
TTTCTTACCTAGACCAGTGGATCCAGAAAGAGTTCCAGAATTGTACAAGGACCTACTGATGTACACTC
TCGAG

| | | | |
|---|---|---|---|
| FGF-CX1g, 316351224 | SEQ ID NO: 14 | 183 aa | MW at 20632.2kD |
| Protein Sequence | | | |

RSMAPLAEVGGFLGGLEGLGQPGAAQLAHLHGILRRRQLYCRTGFHLQILPDGSVQGTRQDHSLFGIL
EFISVAVGLVSIRGVDSGLYLGMNDKGELYGSEKLTSECIFREQFEENWYNTYSSNIYKHGDTGRRYF
VALNKDGTPRDGARSKRHQKFTHFLPRPVDPERVPELYKDLLMYTLE

| | | |
|---|---|---|
| FGF-CX1h, 317459553 | SEQ ID NO: 15 | 408 bp |
| DNA Sequence | ORF Start: | ORF Stop: |

AGATCTATCCTGCGCCGCCGGCAGCTCTATTGCCGCACCGGCTTCCACCTGCAGATCCTGCCCGACGG
CAGCGTGCAGGGCACCCGGCAGGACCACAGCCTCTTCGGTATCTTGGAATTCATCAGTGTGGCAGTGG
GACTGGTCAGTATTAGAGGTGTGGACAGTGGTCTCTATCTTGGAATGAATGACAAAGGAGAACTCTAT
GGATCAGAGAAACTTACTTCCGAATGCATCTTTAGGGAGCAGTTTGAAGAGAACTGGTATAACACCTA
TTCATCTAACATATATAAACATGAAGACACTGGCCGCAGGTATTTTGTGGCACTTAACAAAGACGGAA
CTCCAAGAGATGGCGCCAGGTCCAAGAGGCATCAGAAATTTACACATTTCTTACCTAGACCACTCGAG

| | | | |
|---|---|---|---|
| FGF-CX1h, 317459553 | SEQ ID NO:16 | 1136 aa | MW at 15789.6kD |
| Protein Sequence | | | |

RSILRRRQLYCRTGFHLQILPDGSVQGTRQDHSLFGILEFISVAVGLVSIRGVDSGLYLGMNDKGELY
GSEKLTSECIFREQFEENWYNTYSSNIYKHEDTGRRYFVALNKDGTPRDGARSKRHQKFTHFLPRPLE

| | | |
|---|---|---|
| FGF-CX1i, 317459571 | SEQ ID NO: 17 | 408 bp |
| DNA Sequence | ORF Start: 1 | ORF Stop: |

AGATCTATCCTGCGCCGCCGGCAGCTCTATTGCCGCACCGGCTTCCACCTGCAGATCCTGCCCGACGG
CAGCGTGCAGGGCACCCGGCAGGACCACAGCCTCTTCGGTATCTTGGAATTCATCAGTGTGGCAGTGG
GACTGGTCAGTATTAGAGGTGTGGACAGTGGTCTCTATCTTGGAATGAATGACAAAGGAGAACTCTAT
GGATCAGAGAAACTTACTTCCGAATGCATCTTTAGGGAGCAGTTTGAAGAGAACTGGTATAACACCTA
TTCATCTAACATATATAAACATGGAGACACTGGCCGCAGGTATTTTGTGGCACTTAACAAAGACGGAA
CTCCAAGAGATGGCGCCAGGTCCAAGAGGCATCAGAAATTTACACATTTCTTACCTAGACCACTCGAG

| | | | |
|---|---|---|---|
| FGF-CX1i, 317459571 | SEQ ID NO: 18 | 136 aa | MW at 15717.6kD |
| Protein Sequence | | | |

RSILRRRQLYCRTGFHLQILPDGSVQGTRQDHSLFGILEFISVAVGLVSIRGVDSGLYLGMNDKGELY
GSEKLTSECIFREQFEENWYNTYSSNIYKHGDTGRRYFVALNKDGTPRDGARSKRHQKFTHFLPRPLE

| | | |
|---|---|---|
| FGF-CX1j, CG53135-02 | SEQ ID NO: 19 | 477 bp |
| DNA Sequence | ORF Start: ATG at 1 | ORF Stop: end of sequence |

ATGGCTCAGCTGGCTCACCTGCATGGTATCCTGCGTCGCCGTCAGCTGTACTGCCGTACTGGTTTCCA
CCTGCAGATCCTGCCGGATGGTTCTGTTCAGGGTACCCGTCAGGACCACTCTCTGTTCGGTATCCTGG
AATTCATCTCTGTTGCTGTTGGTCTGGTTTCTATCCGTGGTGTTGACTCTGGCCTGTACCTGGGTATG
AACGACAAAGGCGAACTGTACGGTTCTGAAAAACTGACCTCTGAATGCATCTTCCGTGAACAGTTTGA
AGAGAACTGGTACAACACCTACTCTTCCAACATCTACAAACATGGTGACACCGGCCGTCGCTACTTCG
TTGCTCTGAACAAAGACGGTACCCCGCGTGATGGTGCTCGTTCTAAACGTCACCAGAAATTCACCCAC
TTCCTGCCGCGCCCAGTTGACCCGGAGCGTGTTCCAGAACTGTATAAAGACCTGCTGATGTACACCTA
A

| | | | |
|---|---|---|---|
| FGF-CX1j, CG53135-02 | SEQ ID NO: 20 | 158 aa | MW at 18254.6kD |
| Protein Sequence | | | |

MAQLAHLHGILRRRQLYCRTGFHLQILPDGSVQGTRQDHSLFGILEFISVAVGLVSIRGVDSGLYLGM
NDKGELYGSEKLTSECIFREQFEENWYNTYSSNIYKHGDTGRRYFVALNKDGTPRDGARSKRHQKFTH
FLPRPVDPERVPELYKDLLMYT

| | | |
|---|---|---|
| FGF-CX1k, CG53135-03 | SEQ ID NO: 21 | 636 bp |
| DNA Sequence | ORF Start: ATG at 1 | ORF Stop: end of sequence |

ATGGCTCCCTTAGCCGAAGTCGGGGGCTTTCTGGGCGGCCTGGAGGGCTTGGGCCAGCAGGTGGGTTC
GCATTTCCTGTTGCCTCCTGCCGGGGAGCGGCCGCCGCTGCTGGGCGAGCGCAGGAGCGCGGCGGAGC
GGAGCGCGCGCGGCGGGCCGGGGGCTGCGCAGCTGGCGCACCTGCACGGCATCCTGCGCCGCCGGCAG
CTCTATTGCCGCACCGGCTTCCACCTGCAGATCCTGCCCGACGGCAGCGTGCAGGGCACCCGGCAGGA
CCACAGCCTCTTCGGTATCTTGGAATTCATCAGTGTGGCAGTGGGACTGGTCAGTATTAGAGGTGTGG
ACAGTGGTCTCTATCTTGGAATGAATGACAAAGGAGAACTCTATGGATCAGAGAAACTTACTTCCGAA
TGCATCTTTAGGGAGCAGTTTGAAGAGAACTGGTATAACACCTATTCATCTAACATATATAAACATGG
AGACACTGGCCGCAGGTATTTTGTGGCACTTAACAAAGACGGAACTCCAAGAGATGGCGCCAGGTCCA
AGAGGCATCAGAAATTTACACATTTCTTACCTAGACCAGTGGATCCAGAAAGAGTTCCAGAATTGTAC
AAGGACCTACTGATGTACACTTGA

| | | | |
|---|---|---|---|
| FGF-CX1k, CG53135-03 | SEQ ID NO: 22 | 211 aa | MW at 23498.4kD |
| Protein Sequence | | | |
| MAPLAEVGGFLGGLEGLGQQVGSHFLLPPAGERPPLLGERRSAAERSARGGPGAAQLHLHGILRRRQ | | | |
| LYCRTGFHLQILPDGSVQGTRQDHSLFGILEFISVAVGLVSIRGVDSGLYLGMNDKGELYGSEKLTSE | | | |
| CIFREQFEENWYNTYSSNIYKHGDTGRRYFVALNKDGTPRDGARSKRHQKFTHFLPRPVDPERVPELY | | | |
| KDLLMYT | | | |

| | | |
|---|---|---|
| FGF-CX1l, CG53135-06 | SEQ ID NO: 23 | 540 bp |
| DNA Sequence | ORF Start: ATG at 1 | ORF Stop: end of sequence |

ATGGCTCCCTTAGCCGAAGTCGGGGGCTTTCTGGGCGGCCTGGAGGGCTTGGGCCAGCCGGGGGCAGC
GCAGCTGGCGCACCTGCACGGCATCCTGCGCCGCCGGCAGCTCTATTGCCGCACCGGCTTCCACCTGC
AGATCCTGCCCGACGGCAGCGTGCAGGGCACCCGGCAGGACCACAGCCTCTTCGGTATCTTGGAATTC
ATCAGTGTGGCAGTGGGACTGGTCAGTATTAGAGGTGTGGACAGTGGTCTCTATCTTGGAATGAATGA
CAAAGGAGAACTCTATGGATCAGAGAAACTTACTTCCGAATGCATCTTTAGGGAGCAGTTTGAAGAGA
ACTGGTATAACACCTATTCATCTAACATATATAAACATGGAGACACTGGCCGCAGGTATTTTGTGGCA
CTTAACAAAGACGGAACTCCAAGAGATGGCGCCAGGTCCAAGAGGCATCAGAAATTTACACATTTCTT
ACCTAGACCAGTGGATCCAGAAAGAGTTCCAGAATTGTACAAGGACCTACTGATGTACACTTAG

| | | | |
|---|---|---|---|
| FGF-CX1l, CG53135-06 | SEQ ID NO: 24 | 179 aa | MW at 20146.7kD |
| Protein Sequence | | | |

MAPLAEVGGFLGGLEGLGQPGAAQLAHLHGILRRRQLYCRTGFHLQILPDGSVQGTRQDHSLFGILEF
ISVAVGLVSIRGVDSGLYLGMNDKGELYGSEKLTSECIFREQFEENWYNTYSSNIYKHGDTGRRYFVA
LNKDGTPRDGARSKRHQKFTHFLPRPVDPERVPELYKDLLMYT

| | | |
|---|---|---|
| FGF-CX1m, CG53135-07 | SEQ ID NO: 25 | 54 bp |
| DNA Sequence | ORF Start: ATG at 1 | ORF Stop: |

ATGGCTCCCTTACCCGAAGTCGGGGGCTTTCTGGGCGGCCTGGAGGGCTTGGGC

| | | | |
|---|---|---|---|
| FGF-CX1m, CG53135-07 | SEQ ID NO: 26 | 18 aa | MW at 1688.0kD |
| Protein Sequence | | | |

MAPLAEVGGFLGGLEGLG

| | | |
|---|---|---|
| FGF-CX1n, CG53135-08 | SEQ ID NO: 27 | 63 bp |
| DNA Sequence | ORF Start: | ORF Stop: |

GAGCGGCCGCCGCTGCTGGGCGAGCGCAGGAGCGCGGCGGAGCGGAGCGCGCGCGGCGGGCCG

| | | | |
|---|---|---|---|
| FGF-CX1n, CG53135-08 | SEQ ID NO: 28 | 21 aa | MW at 2262.5kD |
| Protein Sequence | | | |

ERPPLLGERRSAAERSARGGP

| | | |
|---|---|---|
| FGF-CX1o, CG53135-09 | SEQ ID NO: 29 | 63 bp |
| DNA Sequence | ORF Start: | ORF Stop: |

CGCAGGTATTTTGTGGCACTTAACAAAGACGGAACTCCAAGAGATGGCGCCAGGTCCAAGAGG

| | | | |
|---|---|---|---|
| FGF-CX1o, CG53135-09 | SEQ ID NO: 30 | 21 aa | MW at 2463.8kD |
| Protein Sequence | | | |

RRYFVALNKDGTPRDGARSKR

| | | |
|---|---|---|
| FGF-CX1p, CG53135-10 | SEQ ID NO: 31 | 160 bp |
| DNA Sequence | ORF Start: | ORF Stop: |

CCTAGACCAGTGGATCCAGAAGAGTTCCAGAATTGTACAAGGACCTACTGATGTACACT

| | | | |
|---|---|---|---|
| FGF-CX1p, CG53135-10 | SEQ ID NO: 32 | 20 aa | MW at 2431.8kD |
| Protein Sequence | | | |

PRPVDPERVPELYKDLLMYT

| | | |
|---|---|---|
| FGF-CX1q, CG53135-11 | SEQ ID NO: 33 | 51 bp |
| DNA Sequence | ORF Start: ATG at 1 | ORF Stop: |

ATGAACGACAAGGGCGAGCTGTACGGCAGCGAGAAGCTGACCAGCGAGTGC

| | | | |
|---|---|---|---|
| FGF-CX1q, CG53135-11 | SEQ ID NO: 34 | 17 aa | MW at 1904.1kD |
| Protein Sequence | | | |

MNDKGELYGSEKLTSEC

| | | |
|---|---|---|
| FGF-CX1r, CG53135-12 | SEQ ID NO: 35 | 633 bp |
| DNA Sequence | ORF Start: ATG at 1 | ORF Stop: |

ATGGCTCCCTTAGCCGAAGTCGGGGGCTTTCTGGGCGGCCTGGAGGGCTTGGGCCAGCAGGTGGGTTc
GCATTTCCTGTTGCCTCCTGCCGGGGAGCGGCCGCCGCTGCTGGGCGAGCGCAGGAGCGCGGCGGAGC
GGAGCGCGCGCGGCGGGCCGGGGGCTGCGCAGCTGGCGCACCTGCACGGCATCCTGCGCCGCCGGCAG
CTCTATTGCCGCACCGGCTTCCACCTGCAGATCCTGCCCGACGGCAGCGTGCAGGGCACCCGGCAGGA
CCACAGCCTCTTCGGTATCTTGGAATTCATCAGTGTGGCAGTGGGACTGGTCAGTATTAGAGGTGTGG
ACAGTGGTCTCTATCTTGGAATGAATGACAAAGGAGAACTCTATGGATCAGAGAAACTTACTTCCGAA
TGCATCTTTAGGGAGCAGTTTGAAGAGAACTGGTATAACACCTATTCATCTAACATATATAAACATGG
AGACACTGGCCGCAGGTATTTTGTGGCACTTAACAAAGACGGAACTCCAAGAGATGGCGCCAGGTCCA
AGAGGCATCAGAAATTTACACATTTCTTACCTAGACCAGTGGATCCAGAAAGAGTTCCAGAATTGTAC
AAGAACCTACTGATGTACACT

| | | | |
|---|---|---|---|
| FGF-CX1r, CG53135-12 | SEQ ID NO: 36 | 211 aa | MW at 23497.4kD |
| Protein Sequence | | | |

MAPLAEVGGFLGGLEGLGQQVGSHFLLPPAGERPPLLGERRSAAERSARGGPGAAQLAHLHGILRRRQ
LYCRTGFHLQILPDGSVQGTRQDHSLFGILEFISVAVGLVSIRGVDSGLYLGMNDKGELYGSEKLTSE
CIFREQFEENWYNTYSSNIYKHGDTGRRYFVALNKDGTPRDGARSKRHQKFTHFLPRPVDPERVPELY
KNLLMYT

A ClustalW comparison of the above protein sequences yields the following sequence alignment shown in Table 1B.

TABLE 1B
Comparison of the FGF-CX1 protein sequences.

| | |
|---|---|
| FGF-CX1a | MAPLAEVGGFLGGLEGLGQQVGSHFLLPPAGERPPLLGERRSAAERSARGGPGAAQLAHL |
| FGF-CX1b | MAPLAEVGGFLGGLEGLGQQVGSHFLLPPAGERPPLLGERRSAAERSARGGPGAAQLAHL |
| FGF-CX1c | ------------------------------------------------------------ |
| FGF-CX1d | ------------------------------------------------------------ |
| FGF-CX1e | -----------------------------------------------------------T |
| FGF-CX1f | -------------------------------TRSILRRRQLYCRTGFHLQILPDGSVQGT |
| FGF-CX1g | ------------------------------------------------------------ |
| FGF-CX1h | ------------------------------------------------------------ |
| FGF-CX1i | ------------------------------------------------------------ |
| FGF-CX1j | -------------------------MAQLAHLHGILRRRQLYCRTGFHLQILPDGSVQGT |
| FGF-CX1k | MAPLAEVGGFLGGLEGLGQQVGSHFLLPPAGERPPLLGERRSAAERSARGGPGAAQLAHL |
| FGF-CX1l | ----MAPLAEVGGFLGGLEGLGQPGAAQLAHLHGILRRRQLYCRTGFHLQILPDGSVQGT |
| FGF-CX1m | ------------------------------------------------------------ |
| FGF-CX1n | ------------------------------------------------------------ |
| FGF-CX1o | ------------------------------------------------------------ |
| FGF-CX1p | ------------------------------------------------------------ |
| FGF-CX1q | ------------------------------------------------------------ |
| FGF-CX1r | MAPLAEVGGFLGGLEGLGQQVGSHFLLPPAGERPPLLGERRSAAERSARGGPGAAQLAHL |
| FGF-CX1a | HGILRRRQLYCRTGFHLQILPDGSVQGTRQDHSLFGILEFISVAVGLVSIRGVDSGLYLG |
| FGF-CX1b | HGILRRRQLYCRTGFHLQILPDGSVQGTRQDHSLFGILEFISVAVGLVSIRGVDSGLYLG |
| FGF-CX1c | ------------------------------------------------------------ |
| FGF-CX1d | ------------------------------------------------------------ |
| FGF-CX1e | RSILRRRQLYCRTGFHLQILPDGSVQGTRQDHSLFGILEFISVAVGLVSIRGVDSGLYLG |
| FGF-CX1f | RQDHSLFGILEFISVAVGLVSIRGVDSGLYLGMNDKGELYGSEKLTSECIFREQFEENWY |
| FGF-CX1g | ------------------------------------------------------------ |
| FGF-CX1h | RSILRRRQLYCRTGFHLQILPDGSVQGTRQDHSLFGILEFISVAVGLVSIRGVDSGLYLG |
| FGF-CX1i | RSILRRRQLYCRTGFHLQILPDGSVQGTRQDHSLFGILEFISVAVGLVSIRGVDSGLYLG |
| FGF-CX1j | RQDHSLFGILEFISVAVGLVSIRGVDSGLYLGMNDKGELYCSEKLTSECIFREQFEENWY |
| FGF-CX1k | HGILRRRQLYCRTGFHLQILPDGSVQGTRQDHSLFGILEFISVAVGLVSIRGVDSGLYLG |
| FGF-CX1l | RQDHSLFGILEFISVAVGLVSIRGVDSGLYLGMNDKGELYGSEKLTSECIFREQFEENWY |
| FGF-CX1m | ------------------------------------------------------------ |
| FGF-CX1n | ------------------------------------------------------------ |
| FGF-CX1o | ------------------------------------------------------------ |
| FGF-CX1p | ------------------------------------------------------------ |
| FGF-CX1q | ------------------------------------------------------------ |
| FGF-CX1r | HGILRRRQLYCRTGFHLQILPDGSVQGTRQDHSLFGILEFISVAVGLVSIRGVDSGLYLG |
| FGF-CX1a | MNDKGELYGSEKLTSECIFREQFEENWYNTYSSNIYKHGDTGRRYFVALNKDGTPRDGAR |
| FGF-CX1b | MNDKGELYGSEKLTSECIFREQFEENWYNTYSSNIYKHGDTGRRYFVALNKDGTPRDGAR |
| FGF-CX1c | ---------------------------------------------MAPLAEVGGFLGGLE |
| FGF-CX1d | ------------------------------------------TRSMAPLAEVGGFLGGLE |
| FGF-CX1e | MNDKGELYGSEKLTSECIFREQFEENWYNTYSSNIYKHGDTGRRYFVALNKDGTPRDGAR |
| FGF-Cx1f | NTYSSNIYKHGDTGRRYFVALNKDGTPRDGARSKRHQKFTHFLPRPVDPERVPELYKDLL |
| FGF-CX1g | -------------------------------------------RSMAPLAEVGGFLGGLE |
| FGF-CX1h | MNDKGELYGSEKLTSECIFREQFEENWYNTYSSNIYKHEDTGRRYFVALNKDGTPRDGAR |
| FGF-CX1i | MNDKGELYGSEKLTSECIFREQFEENWYNTYSSNIYKHGDTGRRYFVALNKDGTPRDGAR |
| FGF-CX1j | NTYSSNIYKHGDTGRRYFVALNKDGTPRDGARSKRHQKFTHFLPRPVDPERVPELYKDLL |
| FGF-CX1k | MNDKGELYGSEKLTSECIFREQFEENWYNTYSSNIYKHGDTGRRYFVALNKDGTPRDGAR |
| FGF-CX1l | NTYSSNIYKHGDTGRRYFVALNKDGTPRDGARSKRHQKFTHFLPRPVDPERVPELYKDLL |
| FGF-CX1m | ---------------------------------------------MAPLAEVGGFLGGLE |
| FGF-CX1n | ------------------------------------------ERPPLLGERRSAAERSAR |
| FGF-CX1o | ------------------------------------------RRYFVALNKDGTPRDGAR |
| FGF-CX1p | -------------------------------------------PRPVDPERVPELYKDLL |
| FGF-CX1q | ------------------------------------------------MNDKGELYGSEK |
| FGF-CX1r | MNDKGELYGSEKLTSECIFREQFEENWYNTYSSNIYKHGDTGRRYFVALNKDGTPRDGAR |
| FGF-CX1a | SKRHQKFTHFLPRPVDPERVPELYKDLLMYT----------------------------- |
| FGF-CX1b | SKRHQKFTHFLPRPVDPERVPELYKDLLMYT----------------------------- |
| FGF-CX1c | GLGQPGAAQLAHLHGILRRRQLYCRTGFHLQILPDGSAQGTRQDHSLFGILEFISVAVGL |
| FGF-CX1d | GLGQPGAAQLAHLHGILRRRQLYCRTGFHLQILPDGSVQGTRQDHSLFGILEFISVAVGL |
| FGF-CX1e | SKRHQKFTHFLPRPVDG------------------------------------------- |
| FGF-CX1f | MYTVDG------------------------------------------------------ |
| FGF-CX1g | GLGQPGAAQLAHLNGILRRRQLYCRTGFHLQILPDGSVQGTRQDHSLFGILEFISVAVGL |
| FGF-CX1h | SKRHQKFTHFLPRPLE-------------------------------------------- |
| FGF-CX1i | SKRHQKFTHFLPRPLE-------------------------------------------- |
| FGF-CX1j | MYT--------------------------------------------------------- |
| FGF-CX1k | SKRHQKFTHFLPRPVDPERVPELYKDLLMYT----------------------------- |
| FGF-CX1l | MYT--------------------------------------------------------- |
| FGF-CX1m | GLG--------------------------------------------------------- |
| FGF-CX1n | GGP--------------------------------------------------------- |
| FGF-CX1o | SKR--------------------------------------------------------- |
| FGF-CX1p | MYT--------------------------------------------------------- |
| FGF-CX1q | LTSEC------------------------------------------------------- |
| FGF-CX1r | SKRHQKFTHFLPRPVDPERVPELYKNLLMYT----------------------------- |
| FGF-CX1a | ------------------------------------------------------------ |
| FGF-CX1b | ------------------------------------------------------------ |
| FGF-CX1c | VSIRGVDSGLYLGMNDKGELYGSEKLTSECIFREQFEENWYNTYSSNIYKHGDTGRRYFV |
| FGF-CX1d | VSIRGVDSGLYLGMNDKGELYGSEKLTSECIFREQFEENWYNTYSSNIYKHGDTGRRYFV |
| FGF-CX1e | ------------------------------------------------------------ |
| FGF-CX1f | ------------------------------------------------------------ |
| FGF-CX1g | VSIRGVDSGLYLGMNDKGELYGSEKLTSECIFREQFEENWYNTYSSNIYKHGDTGRRYFV |
| FGF-CX1h | ------------------------------------------------------------ |
| FGF-CX1i | ------------------------------------------------------------ |
| FGF-CX1j | ------------------------------------------------------------ |
| FGF-CX1k | ------------------------------------------------------------ |
| FGF-CX1l | ------------------------------------------------------------ |
| FGF-CX1m | ------------------------------------------------------------ |
| FGF-CX1n | ------------------------------------------------------------ |
| FGF-CX1o | ------------------------------------------------------------ |
| FGF-CX1p | ------------------------------------------------------------ |
| FGF-CX1q | ------------------------------------------------------------ |
| FGF-CX1r | ------------------------------------------------------------ |
| FGF-CX1a | ----------------------------------------------- |
| FGF-CX1b | ----------------------------------------------- |
| FGF-CX1c | ALNKDGTPRDGARSKRHQKFTHFLPRPVDPERVPELYKDLLMYT--- |
| FGF-CX1d | ALNKDGTPRDGARSKRHQKFTHFLPRPVDPERVPELYKDLLMYTVDG |
| FGF-CX1e | ----------------------------------------------- |
| FGF-CX1f | ----------------------------------------------- |
| FGF-CX1g | ALNKDGTPRDGARSKRHQKFTHFLPRPVDPERVPELYKDLLMYTLE- |
| FGF-CX1h | ----------------------------------------------- |
| FGF-CX1i | ----------------------------------------------- |
| FGF-CX1j | ----------------------------------------------- |
| FGF-CX1k | ----------------------------------------------- |
| FGF-CX1l | ----------------------------------------------- |
| FGF-CX1m | ----------------------------------------------- |
| FGF-CX1n | ----------------------------------------------- |
| FGF-CX1o | ----------------------------------------------- |
| FGF-CX1p | ----------------------------------------------- |
| FGF-CX1q | ----------------------------------------------- |
| FGF-CX1r | ----------------------------------------------- |
| FGF-CX1a | (SEQ ID NO: 2) |
| FGF-CX1b | (SEQ ID NO: 4) |
| FGF-CX1c | (SEQ ID NO: 6) |
| FGF-CX1d | (SEQ ID NO: 8) |
| FGF-CX1e | (SEQ ID NO: 10) |
| FGF-CX1f | (SEQ ID NO: 12) |
| FGF-CX1g | (SEQ ID NO: 14) |
| FGF-CX1h | (SEQ ID NO: 16) |
| FGF-CX1i | (SEQ ID NO: 18) |
| FGF-CX1j | (SEQ ID NO: 20) |
| FGF-CX1k | (SEQ ID NO: 22) |
| FGF-CX1l | (SEQ ID NO: 24) |
| FGF-CX1m | (SEQ ID NO: 26) |
| FGF-CX1n | (SEQ ID NO: 28) |
| FGF-CX1o | (SEQ ID NO: 30) |
| FGF-CX1p | (SEQ ID NO: 32) |
| FGF-CX1q | (SEQ ID NO: 34) |
| FGF-CX1r | (SEQ ID NO: 36) |

Further analysis of the FGF-CX1a protein yielded the following properties shown in Table 1C.

TABLE 1C
Protien Sequence Properties FGF-CX1a

| SignalP analysis: | No Known Signal Sequence Indicated |
|---|---|
| PSORT II analysis: | |
| PSG: | a new signal peptide prediction method |
| | N-region: length 6; pos.chg 0; neg.chg 1 |
| | H-region: length 8; peak value 0.00 |
| | PSG score: −4.40 |
| GvH: | von Heijne's method for signal seq. recognition |
| | GvH score (threshold: −2.1): −5.49 |
| | possible cleavage site: between 16 and 17 |

>>> Seems to have no N-terminal signal peptide

| | |
|---|---|
| ALOM: | Klein et al's method for TM region allocation |
| | Init position for calculation: 1 |
| | Tentative number of TMS(s) for the threshold 0.5: 1 |
| | Number of TMS(s) for threshold 0.5: 1 |
| | INTEGRAL Likelihood = −6.42 Transmembrane 94-110 |
| | PERIPHERAL Likelihood = 5.20 (at 1) |
| | ALOM score: −6.42 (number of TMSs: 1) |
| MTOP: | Prediction of membrane topology (Hartmann et al.) |
| | Center position for calculation: 101 |
| | Charge difference: 0.5 C(0.0) − N(−0.5) |
| | C > N: C-terminal side will be inside |

>>> membrane topology: type 1b (cytoplasmic tail 94 to 211)

| MITDISC: | discrimination of mitochondrial targeting seq | |
|---|---|---|
| | R content: 0 | Hyd Moment (75): 3.24 |
| | Hyd Moment (95): 6.56 | G content: 4 |
| | D/E content: 2 | S/T content: 0 |
| | Score: −9.30 | |

| | |
|---|---|
| Gavel: | prediction of cleavage sites for mitochondrial preseq |
| | cleavage site motif not found |
| NUCDISC: | discrimination of nuclear localization signals |
| | pat4: none |
| | pat7: none |
| | bipartite: none |
| | content of basic residues: 12.3% |
| | NLS Score: −0.47 |
| KDEL: | ER retention motif in the C-terminus: none |
| | ER Membrane Retention Signals:none |
| SKL: | peroxisomal targeting signal in the C-terminus: none |
| PTS2: | 2nd peroxisomal targeting signal: none |
| VAC: | possible vacuolar targeting motif: none |
| RNA-binding motif: | none |
| Actinin-type actin-binding motif: | |
| | type 1: none |
| | type 2: none |
| NMYR: | N-myristoylation pattern: none |
| Prenylation motif: | none |
| memYQRL: | transport motif from cell surface to Golgi: none |
| Tyrosines in the tail: | too long tail |
| Dileucine motif in the tail: | found |
| | LL at 207 |
| checking 63 PROSITE DNA | none |
| binding motifs: | |
| checking 71 PROSITE ribosomal | none |
| protein motifs: | |
| checking 33 PROSITE prokaryotic | none |
| DNA binding motifs: | |
| NNCN: | Reinhardt's method for Cytoplasmic/Nuclear discrimination |
| | Prediction: cytoplasmic |
| | Reliability: 89 |
| COIL: | Lupas's algorithm to detect coiled-coil regions |
| | total: 0 residues |

```
Final Results (k = 9/23):
34.8%: nuclear
21.7%: mitochondrial
21.7%: cytoplasmic
  8.7%: vesicles of secretory system
  4.3%: vacuolar
  4.3%: peroxisomal
  4.3%: endoplasmic reticulum
>> prediction for CG53135-05 is nuc (k = 23)
```

A search of the FGF-CX1a protein against the Geneseq database, a proprietary database that contains sequences published in patents and patent publication, yielded several homologous proteins shown in Table 1D.

TABLE 1D
Geneseq Results for FGF-CX1a

| | | FGF- | | |
|---|---|---|---|---|
| | | CX1a | Identities/ | |
| | | Residues/ | Similarities for | |
| Geneseq | Protein/Organism/Length | Match | the Matched | Expect |
| Identifier | [Patent#, Date] | Residues | Region | Value |
| ABP54435 | Human fibroblast growth factor | 1 . . . 211 | 211/211 (100%) | e−123 |
| | (FGF) CX protein - Homo sapiens, | 1 . . . 211 | 211/211 (100%) | |
| | 211 aa. [WO200277266-A2, 03-OCT-2002] | | | |
| ABP54434 | Xenopus XFGF-CX amino acid | 1 . . . 211 | 211/211 (100%) | e−123 |
| | sequence SEQ ID NO: 24 - | 1 . . . 211 | 211/211 (100%) | |
| | Xenopus laevis, 211 aa. | | | |
| | [WO200277266-A2, 03-OCT-2002] | | | |
| ABP54429 | Human fibroblast growth factor | 1 . . . 211 | 211/211 (100%) | e−123 |
| | (FGF) CX protein SEQ ID NO: 2 - | 1 . . . 211 | 211/211 (100%) | |
| | Homo sapiens, 211 aa. | | | |
| | [WO200277266-A2, 03-OCT-2002] | | | |
| AAU75323 | Human fibroblast growth factor, | 1 . . . 211 | 211/211 (100%) | e−123 |
| | FGF-CX - Homo sapiens, 211 aa. | 1 . . . 211 | 211/211 (100%) | |
| | [WO200202625-A2, 10-JAN-2002] | | | |
| ABB07261 | Human FGF-20 polypeptide - | 1 . . . 211 | 211/211 (100%) | e−123 |
| | Homo sapiens, 211 aa. | 1 . . . 211 | 211/211 (100%) | |
| | [WO200192522-A2, 06-DEC-2001] | | | |

In a BLAST search of public sequence databases, the FGF-CX1 a protein was found to have homology to the proteins shown in the BLASTP data in Table 1E.

TABLE 1E
Public BLASTP Results for FGF-CX1a

| | | FGF-CX1a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9NP95 | | | | e−122 |
| | (FGF-20) - Homo sapiens | 1 . . . 211 | 211/211 (100%) | |
| | (Human), 211 aa. | | | |
| Q8C7A8 | Fibroblast growth factor 20 - | 1 . . . 211 | 201/211 (95%) | e−117 |
| | Mus musculus (Mouse), 211 aa. | 1 . . . 211 | 204/211 (96%) | |
| Q9EST9 | FGF-20 - Rattus norvegicus | 1 . . . 211 | 201/211 (95%) | e−117 |
| | (Rat), 212 aa. | 1 . . . 211 | 204/211 (96%) | |
| Q9ESL9 | Fibroblast growth factor 20 - | 1 . . . 211 | 200/211 (94%) | e−116 |
| | Mus musculus (Mouse), 212 aa. | 1 . . . 211 | 204/211 (95%) | |
| Q9PVY1 | XFGF-20 - Xenopus laevis | 1 . . . 211 | 170/211 (80%) | 5e−97 |
| | (African clawed frog), 208 aa. | 1 . . . 208 | 189/211 (89%) | |

PFam analysis indicates that the FGF-CX1a protein contains the domains shown in the Table 1F.

TABLE 1F
Domain Analysis of FGF-CX1a

| | | Identities/ | |
|---|---|---|---|
| | FGF-CX1a | Similarities for | |
| Pfam Domain | Match Region | the Matched Region | Expect Value |
| FGF | 63 . . . 194 | 83/147 (56%) | 7.4e−83 |
| | | 122/147 (83%) | |

### Example 2

Identification of Single Nucleotide Polymorphisms in FGF-CX Nucleic Acid Sequences

Variant sequences are also included in this application. A variant sequence can include a single nucleotide polymorphism (SNP). A SNP can, in some instances, be referred to as a “cSNP” to denote that the nucleotide sequence containing the SNP originates as a cDNA. A SNP can arise in several ways. For example, a SNP may be due to a substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, when a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern. Examples include alteration in temporal expression, physiological response regulation, cell type expression regulation, intensity of expression, and stability of transcribed message.

SeqCalling assemblies produced by the exon linking process were selected and extended using the following criteria. Genomic clones having regions with 98% identity to all or part of the initial or extended sequence were identified by BLASTN searches using the relevant sequence to query human genomic databases. The genomic clones that resulted were selected for further analysis because this identity indicates that these clones contain the genomic locus for these SeqCalling assemblies. These sequences were analyzed for putative coding regions as well as for similarity to the known DNA and protein sequences. Programs used for these analyses include Grail, Genscan, BLAST, HMMER, FASTA, Hybrid and other relevant programs.

Some additional genomic regions may have also been identified because selected SeqCalling assemblies map to those regions. Such SeqCalling sequences may have overlapped with regions defined by homology or exon prediction. They may also be included because the location of the fragment was in the vicinity of genomic regions identified by similarity or exon prediction that had been included in the original predicted sequence. The sequence so identified was manually assembled and then may have been extended using one or more additional sequences taken from CuraGen Corporation's human SeqCalling database. SeqCalling fragments suitable for inclusion were identified by the CuraTools™ program SeqExtend or by identifying SeqCalling fragments mapping to the appropriate regions of the genomic clones analyzed.

The regions defined by the procedures described above were then manually integrated and corrected for apparent inconsistencies that may have arisen, for example, from miscalled bases in the original fragments or from discrepancies between predicted exon junctions, EST locations and regions of sequence similarity, to derive the final sequence disclosed herein. When necessary, the process to identify and analyze SeqCalling assemblies and genomic clones was reiterated to derive the full length sequence (Alderborn et al., Determination of Single Nucleotide Polymorphisms by Real-time Pyrophosphate DNA Sequencing. Genome Research. 10 (8) 1249-1265, 2000).

Variants are reported individually but any combination of all or select subset of variants are also included as contemplated NOVX embodiments of the invention.

FGF-CX SNP data

TABLE 2
CG53135-01 (SEQ ID NOs: 3 and 4)

| | Nucleotides | | | Amino Acids | | |
|---|---|---|---|---|---|---|
| Variant | Position | Initial | Modified | Position | Initial | Modified |
| 13377871 | 301 | A | G | 101 | Ile | Val |
| 13375519 | 361 | A | G | 121 | Met | Val |
| 13375518 | 517 | G | A | 173 | Gly | Arg |
| 13375516 | 523 | C | G | 175 | Pro | Ala |
| 13381791 | 616 | G | A | 206 | Asp | Asn |

### Example 3

Derivation of Production Strain

Several different expression constructs were generated to express CG53135 protein (Table 3). CG53135-05 is the protein product that CuraGen produced for toxicology studies and clinical trials. CG53135-05 was expressed in E. coli BLR (DE3) using a codon-optimized, phage-free construct encoding the full-length gene (construct #3).

TABLE 3
Constructs Generated to Express CG53135

| Construct | Construct Description | Construct Diagram |
|---|---|---|
| 1a | NIH 3T3 cells were transfected with pFGF-20, which incorporates an epitope tag (V5) and a polyhistidine tag into the carboxy-terminus of the CG53135-01 protein in the pcDNA3.1 vector (Invitrogen) | 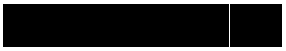 |
| 1b | Human 293-EDNA embryonic kidney cells or NIH 3T3 cells were transfected with CG53135-01 using pCEP4 vector (Invitrogen) containing an IgK signal sequence, multiple cloning sites, a V5 epitope tag, and a polyhistidine tag | 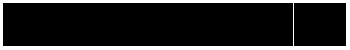 |
| 2 | E. coli BL21 cells were transformed with CG53135-01 using pETMY vector (CuraGen Corporation) containing a polyhistidine tag and a T7 epitope tag (this construct is also referred to as E.coli/pRSET) | 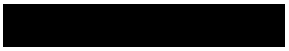 |
| 3 | E. coli BLR (DE3) cells (NovaGen) were transformed with CG53135-05 (full-length, codon-optimized) using pET24a vector (NovaGen) | 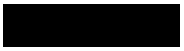 |
| 4 | E. coli BLR (DE3) cells (NovaGen) were transformed with CG53135 (deletion of amino acids 2-54, codon optimized) using pET24a vector (NovaGen) | 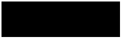 |

Orange = protein sequence;
green = IgK or T7 tag;
red ± V5 tag;
aqua = histidine tag.

Initially, CuraGen cloned the full-length CG53135 gene (CG53135-01) as a Bgl II-Xho I fragment (CG53135-01) into the Bam H1-Xho I sites in mammalian expression vector, pcDNA3.1V5His (Invitrogen Corporation, Carlsbad, Calif.). The resultant vector, pFGF-20 (construct 1a) has a 9 amino acid V5 and a 6 amino acid histidine tag (His) fused in-frame to the carboxy-terminus of CG53135-01. These tags aid in the purification and detection of CG53135-01 protein. After transfection of pFGF-20 into murine NIH 3T3 cells, CG53135-01 protein was detected in the conditioned medium using an anti-V5 antibody (Invitrogen, Carlsbad, Calif.). The full-length CG53135-01 gene was also cloned as a Bgl II-Xho I fragment into the Bam HI-Xho I sites of mammalian expression vector pCEP4/Sec (CuraGen Corporation). This resultant vector, pIgK-FGF-20 (construct 1b) has a heterologous immunoglobulin kappa (IgK) signal sequence that could aid in secretion of CG53135-01. After transfection of pIgK-FGF-20 into human 293 EBNA cells (Invitrogen, Carlsbad, Calif.; catalog # R620-07), CG53135-01 was detected in the conditioned medium using an anti-V5 antibody.

In order to increase the yield of CG53135 protein, CuraGen cloned a Bgl II-Xho I fragment encoding the full-length CG53135-01 gene into the Bam HI-Xho I sites of E. coli expression vector, pETMY (CuraGen Corporation). The resultant vector, pETMY-FGF-20 (construct 2) has a 6 amino acid histidine tag and an amino acid T7 tag fused in-frame to the amino terminus of CG53135. After transformation of pETMY-FGF-20 into BL21 E. coli (Novagen, Madison, Wis.), followed by T7 RNA polymerase induction, CG53135-01 protein was detected in the soluble fraction of the cells.

In order to express CG53135 without tags, a codon-optimized, full-length (CG53135-05) and a codon-optimized deletion construct (CG53135-02, with the N-terminal amino acids 2-54 removed) were synthesized. For the full-length construct, an Nde I restriction site (CATATG) containing the initiator codon was placed at the 5′ end of the coding sequence. At the 3′ end, the coding sequence was followed by 2 consecutive stop codons (TAA) and a Xho restriction site (CTCGAG). The synthesized gene was cloned into pCRScript (Stratagene, La Jolla, Calif.) to generate pCRScript-CG53135. An Nde I-Xho I fragment containing the codon-optimized CG53135 gene was isolated from the pCRscript-CG53135 and subcloned into Nde I-Xho I-digested pET24a to generate pET24a-CG53135 (construct 3). The full-length, codon-optimized version of CG53135 is referred to as CG53135-05.

To generate a codon-optimized deletion construct for CG53135, oligonucleotide primers were designed to amplify the deleted CG53135 gene from pCRScript-CG53135. The forward primer contained an Nde I site (CATATG) followed by coding sequence starting at amino acid 55. The reverse primer contained a HindIII restriction site. A single PCR product of approximately 480 base pairs was obtained and cloned into pCR2.1 vector (Invitrogen) to generate pCR2.1-CG53135del. An Nde I-Hind III fragment was isolated from pCR2.1-53135del and subcloned into Nde I-Hind III-digested pET24a to generate pET24a-CG53135-02 (construct 4).

The plasmids, pET24a-CG53135-05 (construct 3) and pET24a-CG53135-02 (construct 4) have no tags. Each vector was transformed into E. coli BLR (DE3), induced with isopropyl thiogalactopyranoside, and full-length or N-terminally truncated CG53135 protein was detected in the soluble fraction of cells.

Section II

Oral Mucositis

### BACKGROUND

Mucositis, inflammation and ulceration of the mucosa of the alimentary tract, is often induced by radiation therapy (RT) or chemotherapy (CT). Specific anatomic locations vulnerable to mucositis include the oral and nasopharyngeal cavity, the esophagus, the small intestine and the rectum. Oral ulcerative mucositis is a common, painful, dose-limiting toxicity of drug and radiation therapy for cancer. The disorder is characterized by breakdown of the oral mucosa that results in the formation of ulcerative lesions. In myelosuppressed patients, the ulcerations that accompany mucositis are frequent portals of entry for indigenous oral bacteria often leading to sepsis or bacteremia. Candida, for example, is one such indigenous organism found orally, which is capable of producing opportunistic infections within the oral cavity when appropriate predisposing factors exist. Mucositis occurs to some degree in more than one-third of patients receiving antineoplastic drug therapy. The frequency and severity are significantly greater among patients who are treated with induction therapy for leukemia or with many of the conditioning regimens for bone marrow transplant. Among these individuals, moderate to severe mucositis is not unusual in more than three-quarters of patients. Moderate to severe mucositis occurs in virtually all patients who receive radiation therapy for tumors of the head and neck and typically begins with cumulative exposures of 15 Gy and then worsens as total doses of 60 Gy or more are reached (Peterson DE, Curr Opin Oncol 1999 11:261-6; Plevova P, Oral Oncol 1999 35:453-70; Knox JJ et al., Drugs Aging 2000 17:257-67; Sonis ST et al., J Clin Oncol 2001 19:2201-5).

Clinically, mucositis progresses through three stages. In Stage 1, inflammation is accompanied by painful mucosal erythema, which can respond to local anesthetics. In Stage 2, there is painful ulceration with pseudomembrane formation and the pain is often of such intensity as to require parenteral narcotic analgesia. In the case of myelosuppressive treatment, there is also potentially life-threatening sepsis, requiring antimicrobial therapy. In Stage 3, there is spontaneous healing, occurring about 2-3 weeks after cessation of anti-neoplastic therapy.

Currently, there is no approved treatment for mucositis. Standard therapy for mucositis is predominantly palliative, including application of topical analgesics such as lidocaine and/or systemic administration of narcotics and antibiotics (Peterson DE, Curr Opin Oncol 1999 11:261-6; Plevova P, Oral Oncol 1999 35:453-70; Knox J J et al., Drugs Aging 2000 17:257-67; Sonis S T et al., J Clin Oncol 2001 19:2201-5). Several agents have been evaluated for safety and efficacy in preventing or treating oral mucositis (Peterson DE, Curr Opin Oncol 1999 11:261-6; Plevova P, Oral Oncol 1999 35:453-70; Knox J J et al., Drugs Aging 2000 17:257-67; Rosenthal C et al., Antibiot Chemother 2000 50:115-32; Crawford J et al., Cytokines Cell Mol Ther 1999 5:187-93; Bez C et al., Oral Surg Oral Med Oral Pathol Oral Radiol Endod 1999 88:311-5; Danilenko D M. Toxicol Pathol 1999 27:64-71). These include mucosal protective agents, antibiotics, and growth factors, such as transforming growth factor (TGF), interleukin-11 (IL-11), granulocyte-macrophage colony stimulating factor (GM-CSF), and keratinocyte growth factor (KGF).

The FGF family of signaling molecules consists of more than 20 related growth factors (Szebenyi G and Fallon J F, Int Rev Cytol 1999 185:45-106; Yamashita T et al., Biochem Biophys Res Commun 2000 277:494-8). Common to all members are two conserved cysteine residues and high homology (up to 66% amino acid identity) throughout a core 120 amino acid region that includes the FGF receptor binding region. FGFs are implicated in a wide range of normal developmental processes including cell replication, angiogenesis, apoptosis, cell adhesion, motility and body plan patterning (e.g., gastrulation, neurulation, anteroposterior organization), organogenesis, and development of the limbs (Szebenyi G and Fallon J F, Int Rev Cytol 1999 185:45-106). FGFs are intricately involved in vasculogenesis, cell proliferation, and cell-matrix interactions. Ectopic or inappropriate expression of the FGFs or their receptors can lead to oncogenesis. Because of the ability to regulate cellular growth, proliferation and angiogenesis, FGFs could be useful as therapeutics indicated for diseases characterized by tissue wounding or ulceration.

### SUMMARY OF THE INVENTION

The FGF family, which consists of more than 20 cytokines, includes signaling molecules implicated in normal developmental and physiological processes, such as growth, survival, apoptosis, motility, and differentiation. Utilizing a homology-based genomic DNA mining process, the inventors identified the cDNA for a novel FGF, CG53135. CG53135 was recognized by multiple FGF receptors (primarily FGFR2 and FGFR3) and these receptors are widely expressed in human tissues. In vitro, CG53135 induced proliferation of epithelial and mesenchymal cells but not smooth muscle, erythroid, endothelial, or lymphoid cells.

CG53135 preserves the integrity of the intestinal mucosa and has potential to treat diseases associated with damaged mucosa. CG53135 was active in two models of oral mucositis in hamsters. In radiation-induced mucositis model, CG53135 (3 mg/kg/day topical administration for 18 days or 6-12 mg/kg/day intraperitoneal administration for up to 18 days) reduced the severity of mucositis. In chemotherapy-induced mucositis model, CG53135 (12 mg/kg/day intraperitoneal administration for as few as 2 days) reduced the severity of mucositis.

### Example 4

Cellular Proliferation Responses with CG53135 (Studies L-117.01 and L-117.02)

### BACKGROUND

Novel members of the FGF family could have significant therapeutic potential in diseases associated with cell and tissue remodeling, as these growth factors regulate diverse cellular functions such as growth, survival, apoptosis, motility and differentiation. A number of experiments were performed to characterize the biological activity of the novel human FGF, CG53135. Fibroblast growth factors are known to have both stimulatory and inhibitory effects on a wide variety of cell types. The proliferative response of representative cell types to a full-length tagged variant (CG53135-01), a deletion variant (CG53135-02), and a full-length codon-optimized untagged variant (CG53135-05) of CG53135 was evaluated.

Materials and Methods

Heterologous Protein Expression CG53135-01 (batch 4A and 6) were used in these experiments. Protein was expressed using Escherichia coli (E. coli), BL21 (Novagen, Madison, Wis.), transformed with full-length CG53135-01 in a pETMY-hFGF20X/BL21 expression vector. Cells were harvested and disrupted, and then the soluble protein fraction was clarified by filtration and passed through a metal chelation column. The final protein fraction was dialyzed against phosphate buffered saline (PBS)+1 M L-arginine. Protein samples were stored at −70° C.

CG53135-02, (batch 1 and 13) were used in these experiments. Protein was expressed in E. coli, BLR (DE3) (Novagen), transformed with the deletion variant CG53135-02 inserted into a pET24a vector (Novagen). A research cell bank (RCB) was produced and cell paste containing CG53135-02 was produced by fermentation of cells originating from the RCB. Cell membranes were disrupted by high-pressure homogenization, and lysate was clarified by centrifugation. CG53135-02 was purified by ion exchange chromatography. The final protein fraction was dialyzed against the formulation buffer (100 mM citrate, 1 mM ethylenediaminetetraacetic acid (EDTA), 1 M L-arginine).

CG53135-05, DEV10, used in these experiments, was prepared by Cambrex Biosciences (Hopkinton, Mass.). Recombinant human basic FGF (bFGF) and vascular endothelial growth factor (VEGF) were purchased from R & D Systems (Minneapolis, Minn.). Recombinant human KGF-2, batch 2, used in this study was expressed in E. coli using a codon-optimized, deletion construct (missing amino acids 2-68), which was subcloned into the pQE60 expression vector (QiaGen Corp, Valencia, Calif.). Cells were fermented in the presence of isopropylthiogalactoside (IPTG) to induce expression of KGF-2 protein, as described in the manufacturer's manual.

BrdU Incorporation Proliferative activity was measured by treatment of serum-starved cultured cells with a given agent and measurement of BrdU incorporation during DNA synthesis. Cells were cultured in respective manufacturer recommended basal growth medium supplemented with 10% fetal bovine serum or 10% calf serum as per manufacturer recommendations. Cells were grown in 96-well plates to confluence at 37° C. in 10% CO₂/air (to subclonfluence at 5% CO₂ for dedifferentiated chondrocytes and NHOst). Cells were then starved in respective basal growth medium for 24-72 h. CG53135 protein purified from E. coli or pCEP4/Sec or pCEP4/Sec-FGF20X enriched conditioned medium was added (10 μL/100 μL of culture) for 18 h. BrdU (10 μM final concentration) was then added and incubated with the cells for 5 h. BrdU incorporation was assayed according to the manufacturer's specifications (Roche Molecular Biochemicals, Indianapolis, Ind.).

Growth Assay Growth activity was obtained by measuring cell number following treatment of cultured cells with a given agent for a specified period of time. In general, cells grown to ˜20% confluency in 6-well dishes were treated with basal medium supplemented with CG53135 or control, incubated for several days, trypsinized and counted using a Coulter Z1 Particle Counter.

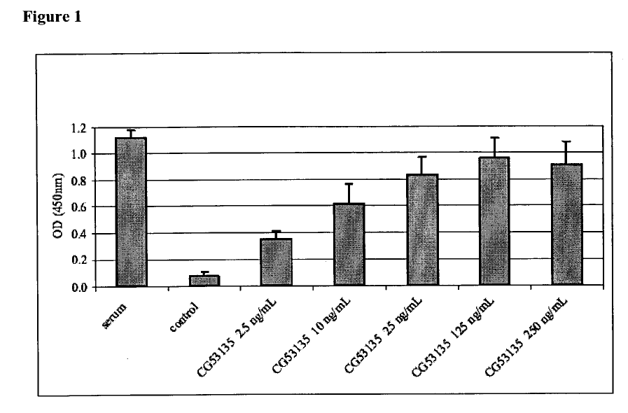

Proliferation in Mesenchymal Cells To determine if recombinant CG53135 could stimulate DNA synthesis in fibroblasts, we performed a BrdU incorporation assay in CG53135-01 treated NIH 3T3 murine embryonic lung fibroblasts. Recombinant CG53135-01 induced DNA synthesis in NIH 3T3 mouse fibroblasts (FIG. 1) in a dose-dependent manner. DNA synthesis was generally induced at a half maximal concentration of ˜10 ng/mL. In contrast, treatment with vehicle control purified from cells did not induce any DNA synthesis.

CG53135-01 also induced DNA synthesis in other cells of mesenchymal origin, including CCD-1070Sk normal human foreskin fibroblasts, MG-63 osteosarcoma cell line, and rabbit synoviocyte cell line, HIG-82 (data not shown). In contrast, CG53135-01 did not induce any significant increase in DNA synthesis in primary human osteoblasts (NHOst), human pulmonary artery smooth muscle cells, human coronary artery smooth muscle cells, human aorta smooth muscle cells (HSMC), or in mouse skeletal muscle cells (data not shown).

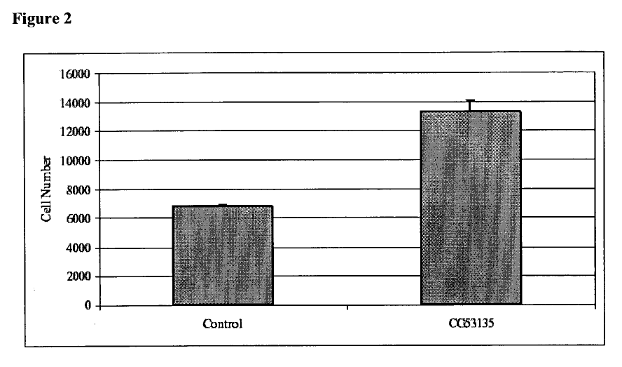

To determine if recombinant CG53135-01 sustained cell growth, NIH 3T3 cells were cultured with 1 μg CG53135-01 or control for 48 hours and then counted (FIG. 2). CG53135 induced an approximately 2-fold increase in cell number relative to control in this assay. These results show that CG53135 acts as a growth factor.

Proliferation of Epithelial Cells To determine if recombinant CG53135 could stimulate DNA synthesis and sustain cell growth in epithelial cells, a BrdU incorporation assay was performed in representative epithelial cell lines treated with CG53135. Cell counts following protein treatment were also determined for some cell lines.

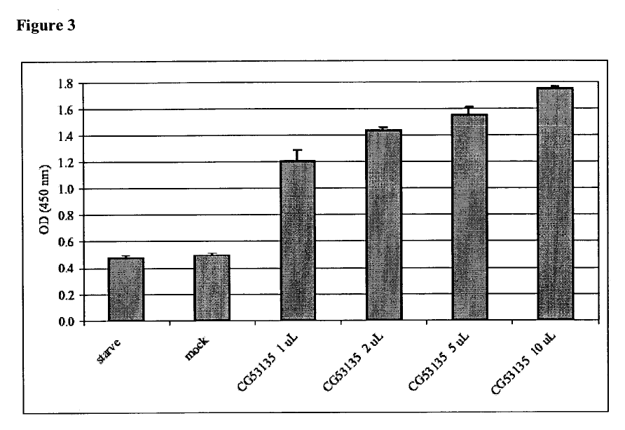

CG53135 was found to induce DNA synthesis in the 786-O human renal carcinoma cell line in a dose-dependent manner (FIG. 3). In addition, CG53135-01 induced DNA synthesis in other cells of epithelial origin, including CCD 1106 KERTr human keratinocytes, Balb MK mouse keratinocytes, and breast epithelial cell line, B5589 (data not shown).

Proliferation of Hematopoietic Cells No stimulatory effect on DNA synthesis was observed upon treatment of TF-1, an erythroblastic leukemia cell line with CG53135-01 (data not shown). These data suggest that CG53135-01 does not induce proliferation in cells of erythroid origin. In addition, Jurkat, an acute T-lymphoblastic leukemia cell line did not show any response when treated with CG53135-01, whereas a robust stimulation of BrdU incorporation was observed with serum treatment (data not shown).

Effects of CG53135 on Endothelial Cells Protein therapeutic agents may inhibit or promote angiogenesis, the process through which endothelial cells differentiate into capillaries. Because CG53135 belongs to the fibroblast growth factor family, some members of which have angiogenic properties, the antiangiogenic or pro-angiogenic effects of CG53135 on endothelial cell lines. The following cell lines were chosen because they are cell types used in understanding angiogenesis in cancer: HUVEC (human umbilical vein endothelial cells), BAEC (bovine aortic endothelial cells), HMVEC-d (human endothelial, dermal capillary). These endothelial cell types undergo morphogenic differentiation and are representative of large vessel (HUVEC, BAEC) as well as capillary endothelial cells (HMVEC-d).

CG53135-01 treatment did not alter cell survival or have stimulatory effects on BrdU incorporation in human umbilical vein endothelial cells, human dermal microvascular endothelial cells or bovine aortic endothelial cells (data not shown). Furthermore, CG53135-01 treatment did not inhibit tube formation, an important event in form ation of new blood vessels, in HUVECS (data not shown); this result suggests that CG53135 does not have anti-angiogenic properties. Finally, CG53135-01 had no effect on VEGF induced cell migration in HUVECs, suggesting that it does no play a role in metastasis (data not shown).

Conclusions

Recombinant CG53135-01 induces a proliferative response in mesenchymal and epithelial cells in vitro (i.e., NIH 3T3 mouse fibroblasts, CCD-1070 normal human skin fibroblasts, CCD-1106 human keratinocytes, 786-O human renal carcinoma cells, MG-63 human osteosarcoma cells and human breast epithelial cells), but not in human smooth muscle, erythroid, or endothelial cells. Similar to CG53135-01, CG53135-02 and CG53135-05 induce proliferation of mesenchymal and epithelial cells (data not shown). In addition, CG53135-02 (but not CG53135-01 nor CG53135-05) induces proliferation of endothelial cells (data not shown).

Since one of the hallmarks of cancer is uncontrolled proliferation, it follows that the FGFs exhibiting mitogenic activity (i.e., FGFs 1-10, 16-18, 20) may be involved in tumorigenesis via direct deregulated growth stimulation of cancer cells in an autocrine, paracrine or juxtacrine fashion. Other FGFs, such as FGF-7 and FGF-10, play a role in regeneration and proliferation, and are currently being developed as protein therapeutics for inflammatory bowel disease, mucositis and wound healing. Therefore, based on the homology of CG53135 with known FGFs, the known properties of the FGF family, and the expression profile of FGF-20, CG53135 is proposed as a potential protein therapeutic in diseases involving epithelial and mesenchymal cell proliferation and regeneration such as inflammatory bowel disease (i.e., ulcerative colitis and Crohn's disease), cancer, mucositis, gastric bleeding & gastric ulcers; tissue injury/wound healing (e.g., spinal cord injury, burns, chronic ulcers) as well as in neurodegenerative diseases such as Alzheimer's disease and Parkinson's disease.

### Example 5

Activity of CG53135 in Hamster Model of Acute Radiation-Induced Oral Mucositis (N-152)

### BACKGROUND

CG53135 is a novel FGF with proliferative effects on epithelial and fibroblastic cell types. We explored whether CG53135 might be active in the treatment of oral mucositis, a disorder with dysfunctional epithelial regeneration. Growth factors, such as CG53135, could prove efficacious in prevention and treatment of oral mucositis by facilitating tissue remodeling and repair processes. Hence, CG53135-05 was evaluated for activity in a hamster model of radiation-induced oral mucositis, and its activity compared with KGF-2, another FGF family member. KGF-2, also referred to as FGF-10, is active in models of wound healing and inflammatory bowel disease (Miceli et al. J Pharmacol Exp Ther 1999, 290:464-71).

The acute radiation model in hamsters (Sonis et al. Oral Surg Oral Med Oral Pathol 1990, 69:437-43) has proven to be an accurate, efficient and cost-effective technique to provide a preliminary evaluation of anti-mucositis compounds, including growth factors and cytokines (Sonis et al. Oral Oncol 2000, 36:373-81; Sonis et al. Cytokine 1997, 9:605-12; Sonis et al. Oral Oncol 1997, 33:47-54). The acute model has little systemic toxicity, resulting in few animal deaths, permitting the use of smaller groups for initial activity studies. It has also been used to study specific mechanistic elements in the pathogenesis of mucositis. Molecules that show activity in the acute radiation model may be further evaluated in the more complex models of fractionated radiation, chemotherapy, or concomitant therapy. In this model, an acute radiation dose of approximately 40 Gy on Day 0 is administered in order to induce severe mucositis. This dose results in predictable ulcerative oral mucositis that typically peaks around Day 16-18.

Materials and Methods

Protein Expression and Purification CG53135-05 used in this study was purified as Batch Dev 08-02. The recombinant human DNA protein, CG53135-05, was expressed using Escherichia coli BLR (DE3) cells (Novagen, Darmstadt, Germany). These cells were transformed with full-length, codon-optimized CG53135-05 using pET24a vector (Novagen). A GMP manufacturing cell bank (MCB) of these cells was produced. Cell paste containing CG53135-05 protein, produced by fermentation of cells originating from the MCB, was lysed with high pressure homogenization in lysis buffer, and clarified by centrifugation. CG53135-05 was purified from clarified cell lysate by 2 cycles of ion exchange chromatography and ammonium sulfate precipitation. The final precipitate was washed with purified water and suspended in formulation buffer as follows: 30 mM citrate (pH 0), 2 mM EDTA, 200 mM sorbitol, 50 mM KCl, 20% glycerin.

Male Golden Syrian hamsters (Charles River Laboratories or Harlan), of age 6 to 7 weeks, and with similar body weight (mean body weight 77.4 g) in all groups at study commencement, were used in this study. Sixty-four hamsters were randomized into 8 groups of 8 animals each prior to irradiation. Each group was assigned a different treatment as shown in Table 4.

TABLE 4
Treatment Groups

| Group | No. of | | Treatment | Volume (mL); |
|---|---|---|---|---|
| No. | Animals | Treatment | Days | Treatment |
| 1 | 8 males | vehicle control IP | Days −5 to −2; | 0.1; once/day |
| | | | 3 to 15 | |
| 2 | 8 males | 300 μg/day CG53135-05 IP | Days 3 to 15 | 0.1; once/day |
| 3 | 8 males | 600 μg/day CG53135-05 IP | Days 3 to 15 | 0.1; once/day |
| 4 | 8 males | 300 μg/day CG53135-05 IP | Days −5 to −2; | 0.1; once/day |
| | | | 3 to 15 | |
| 5 | 8 males | 300 μg/day KGF-2 IP | Days −5 to −2; | 0.125; once/day |
| | | | 3 to 15 | |
| 6 | 8 males | vehicle control topical | Days −5 to −2; | 0.2; three times/day |
| | | | 3 to 15 | |
| 7 | 8 males | 300 μg/day CG53135-05 topical | Days 3 to 15 | 0.2; three times/day |
| 8 | 8 males | 300 μg/day CG53135-05 topical | Days −5 to −2; | 0.2; three times/day |
| | | | 3 to 15 | |

Animals were acutely radiated with a single dose of radiation (40 Gy/dose) on the left buccal mucosa on Day 0. Animals were treated once daily with vehicle or CG53135-05 IP or topically following acute radiation. Animals in Groups 1 to 5 received IP injection of test materials once per day. For Groups 6 to 8, test material was applied topically to the cheek pouch three times per day. The following dosing schedules were used: Day 3 to Day 15 (Groups 2, 3 and 7), and Day -5 to Day -2 then Day 3 to Day 15 (Groups 1, 4, 5, 6 and 8). Doses of CG53135-05 were 300 μg/day (Groups 2, 4, 7 and 8) and 600 mg/day (Group 3). The KGF-2 dose was 300 μg/day (Group 5). Mucositis was evaluated on alternate days beginning on Day 6 and continued until the conclusion of the experiment on Day 28 (i.e., Days 8, 10, 12, 14, 16, 18, 20, 22, 24, 26 & 28). Clinically relevant oral mucositis (i.e., mucositis score of ≧3) developed ˜14 days after radiation.

Each hamster was weighed daily for the period of the study (i.e., Day -5 to Day 28) and its survival recorded in order to assess possible differences in animal weight among treatment groups as an indication for mucositis severity or possible toxicity resulting from the treatments. Mucositis was scored visually by comparison to a validated photographic scale, ranging from 0 for normal, to 5 for severe ulceration; in descriptive terms the clinical scale is defined in Table 5.

TABLE 5
Mucositis Scoring Definitions

| Score: | Description: |
|---|---|
| 0 | Pouch completely healthy. No erythema or vasodilation. |
| 1 | Light to severe erythema and vasodilation. No erosion of mucosa |
| 2 | Severe erythema and vasodilation. Erosion of superficial aspects |
| | of mucosa leaving denuded areas. Decreased stippling of |
| | mucosa. |
| 3 | Formation of off-white ulcers in one or more places. Ulcers may |
| | have a yellow/gray due to pseudomembrane. Cumulative size |
| | of ulcers should equal about ¼ of the pouch. Severe |
| | erythema and vasodilation. |
| 4 | Cumulative size of ulcers should equal about ½ of the pouch. |
| | Loss of pliability. Severe erythema and vasodilation. |
| 5 | Virtually all of pouch is ulcerated. Loss of pliability (pouch can |
| | only partially be extracted from mouth). |

A score of 1-2 is considered to represent a mild stage of the disease, whereas a score of 3-5 is considered to indicate moderate to severe mucositis. Following clinical scoring, a photograph was taken of each animal's mucosa using a standardized technique. At the conclusion of the experiment, all film was developed and the photographs randomly numbered for blinded scoring. Thereafter, 2 independent, trained observers graded the photographs in blinded fashion using the above-described scale. For each photograph the actual blinded score was be based upon the average of the score assigned by the 2 blinded, independent evaluators. Only the scores from blinded photographic evaluation was statistically analyzed and reported in the results.

The effect of each treatment on mucositis compared to the vehicle control group was assessed according to the parameters listed in Table 6.

TABLE 6
Parameters for Evaluation of Activity

| Parameter | Description |
|---|---|
| The difference in the | On each evaluation day, the number of |
| number of days hamsters | animals with a blinded mucositis score of |
| in each group have severe | ≧3 in each drug treatment group was |
| mucositis (score ≧3). | compared to the vehicle control group. |
| | Differences were analyzed on a cumulative |
| | basis. Treatment success was considered a |
| | statistically significant lower number of |
| | hamsters with this score in a drug treatment |
| | group, versus the vehicle control value, as |
| | determined by chi-square analysis. |
| The rank sum differences in | For each evaluation day the scores of the |
| daily mucositis scores. | vehicle control group was compared to those |
| | of the treated group using the non-parametric |
| | rank sum analysis. Treatment success was |
| | considered as a statistically significant |
| | lowering of scores in the treated group on 2 |
| | or more days from Day 6 to Day 28. |

Results

There were no statistically significant differences in survival or weight change over time between the two vehicle control groups and their respective test groups (data not shown).

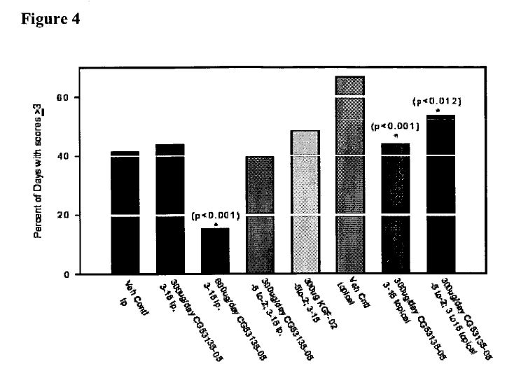

Prophylactic treatment with either 300 μg/animal/day CG53135-05 or KGF-2, administered IP prior to and after radiation (Day -5 to Day -2 then Day 3 to Day 15) failed to elicit significant activity in reducing the incidence of moderate to severe mucositis (FIG. 4). Treatment with 300 μg/animal/day CG53135-05 administered IP from Day 3 to Day 15 also failed to elicit significant activity in reducing the incidence of moderate to severe mucositis (FIG. 4).

Treatment with 600 μg/animal/day CG53135-05 administered IP from Day 3 to Day 15 showed only one day of significant activity by rank sum analysis (p<0.001) (FIG. 4). Though treatment success criteria for this analysis have been defined as two or more days of significant activity, this observation suggests that this treatment has a favorable effect on mucositis. This group also had a statistically significant lower score than corresponding control treatment by chi square analysis (p<0.001). Therefore, this combination of dose, schedule and route of administration is active in treating mucositis in this model.

Treatment with 300 μg/animal/day CG53135-05 administered topically from Day 3 to Day 15 showed significant activity in reducing the incidence of moderate to severe mucositis by Chi square analysis (p<0.001) (data not shown). Treatment was also considered successful by rank sum analysis as mucositis was significantly reduced on five of the twelve scoring days. Therefore, this combination of dose, schedule and route of administration of CG53135-05 has activity in treating mucositis in this model. Treatment success criteria by chi square analysis were met (p=0.012) when 300 μg/animal/day CG53135-05 was administered topically prior to and after radiation (i.e., Day -5 to Day -2 and Day 3 to Day 15). Therefore, this combination of dose, schedule and route of administration of CG53135-05 showed activity in reducing the incidence of moderate to severe mucositis.

In an additional experiment, IP treatment with 300 μg/animal/day CG53135-01 (a tagged, full-length form of CG53135), on Day 3 to 15 also had a beneficial effect on the course and severity of mucositis in the acute radiation model of mucositis in golden Syrian hamsters (data not shown; N-135).

In yet another experiment, untreated control and vehicle-injected control animals were compared with animals treated intraperitoneally with 300, 600, or 1200 μg CG53135-05 (an untagged, full-length form of CG53135 with a slightly different formulation from that used in the experiment above) from Day 3 to Day 15 (data not shown; N-197). No beneficial effect was observed in male animals treated with 300 μg CG53135-05. However, consistent with the results reported above, treatment with 600 μg CG53135-05 resulted in a significant reduction in the severity of mucositis compared with untreated control animals (p<0.001 by Chi-square analysis) and significantly reduced mean daily mucositis scores for 3 of 12 scoring days compared with the vehicle control group. In addition, administration of 1200 μg CG53135-05 significantly reduced the severity of mucositis relative to the vehicle control group (p<0.001 by Chi-square analysis) and significantly reduced mean daily mucositis scores for 5 scoring days. No significant difference in body weight was observed in any of the treatment regimens when compared with the controls.

Conclusions

The activity of CG53135 was evaluated in a model of oral mucositis induced in hamsters administered a single, bolus dose of radiation (40 Gy) on Day 0. Clinically relevant oral mucositis (i.e., mucositis score of ≧3) developed ˜14 days after radiation. In general, treatment with CG53135 therapeutically (i.e., after radiation insult) significantly reduced clinically relevant mucositis, but prophylactic administration of CG53135 (i.e., prior to or concurrent with the insult) had no beneficial effect and could worsen mucositis. Treatment with CG53135 (3 mg/kg/day topical administration for 18 days or 6-12 mg/kg/day intraperitoneal administration for up to 18 days) reduced the severity of mucositis. No studies were conducted using an intravenous (IV) route of administration since IV administration in hamsters is technically challenging and data are consequently highly variable.

### Example 6

Activity of CG53135 in Hamster Model of Chemotherapy-Induced Oral Mucositis (N-212)

### BACKGROUND

Oral mucositis is a painful, dose-limiting toxicity of chemotherapy and radiation therapy for cancer. The disorder is characterized by dysfunctional epithelial regeneration resulting in the breakdown of the oral mucosa and formation of ulcerative lesions. Mucositis occurs to some degree in more than ⅓ of cancer patients receiving chemotherapy. Treatment with CG53135 was shown to be efficacious in the treatment of acute radiation-induced oral mucositis. The mechanism for mucositis development is similar for both radiation therapy and chemotherapy (Peterson DE, Curr Opin Oncol 1999 11:261-6). Therefore, we investigated the potential utility of CG53135 for the treatment of chemotherapy-induced oral mucositis in male Golden Syrian hamsters.

Materials and Methods

Protein Expression and Purification CG53135-05 used in this study (batch 29-NB849:76) was expressed and purified as described in Example 5, with the exception that the final protein fraction was dialyzed against formulation buffer containing 30 mM sodium citrate, 2 mM EDTA, 200 mM sorbitol, 50 mM KCl, 20% glycerol (pH 6.1). Study Design Male golden Syrian hamsters (Charles River Laboratories) age 5 to 6 weeks and with similar body weight in all groups at study commencement, were used in this study. Sixty male hamsters were randomized into 6 groups of 10 animals each prior to irradiation. The treatment groups are outlined in Table 7.

TABLE 7
Treatment Groups

| Group | | |
|---|---|---|
| No. | Treatment (0.1 mL, IP) | Dosing Schedule |
| 1 | Vehicle (Disease Control) | Day 1 to Day 18 |
| 2 | CG53135-05, 12 mg/kg/day | Day 1 to Day 18 |
| 3 | CG53135-05, 12 mg/kg/day | Day 6 to Day 14 |
| 4 | CG53135-05, 12 mg/kg/day | Day 1 to Day 9 |
| 5 | CG53135-05, 12 mg/kg/day | Day 1 to Day 6 |
| 6 | CG53135-05, 12 mg/kg/day | Day 1 to Day 2 |

Mucositis was induced using 5-fluorouracil, delivered as single bolus (60 mg/kg, IP) on Days -4 and -2. A single submucosatoxic dose of radiation (40 Gy/dose) was locally administered to all animals on Day 0. Animals were treated once daily with 0.1 mL vehicle or 12 mg/kg CG53135-05 IP following mucosatoxic insult according to the schedule shown in Table 13. Muscositis was scored visually as described in Example 5 (Table 5) on alternate days beginning on Day 6 and every second day until the conclusion of the experiment on Day 30 (i.e., Days 8, 10, 12, 14, 16, 18, 20, 22, 24, 26, 28, and 30). Each hamster was weighed daily for the period of the study (i.e., Day 0 to Day 30). Weight and survival were monitored as indices for severity of mucositis or possible toxicity resulting from treatment.

The effect of each treatment on mucositis compared with the control group was assessed according to the parameters listed in Table 8. Statistical differences between treatment groups were determined using the Student's t-test, Mann-Whitney U test, and chi-square analysis, with a critical value of 0.05.

TABLE 8
Parameters for evaluation of Activity

| Parameter | Description |
|---|---|
| The difference in the | On each evaluation day, the number of |
| number of days | animals with a blinded mucositis score of |
| hamsters in each group | ≧3 in each drug treatment group was |
| have severe mucositis | compared to the vehicle control group. |
| (score ≧ 3). | Differences were analyzed on a cumulative |
| | basis. Treatment success was considered a |
| | statistically significant lower number of |
| | hamsters with this score in a drug treatment |
| | group, versus the vehicle control value, as |
| | determined by chi-square analysis. |
| The rank sum differences in | For each evaluation day the scores of the |
| daily mucositis scores. | vehicle control group was compared to those |
| | of the treated group using the non-parametric |
| | rank sum analysis. Treatment success was |
| | considered as a statistically significant |
| | lowering of scores in the treated group on 2 |
| | or more days from Day 6 to Day 30. |

Results

There were no statistically significant differences in weight or survival over time between the vehicle control group (Group 1) and CG53135-05 treatment groups (Groups 2-6) (data not shown).

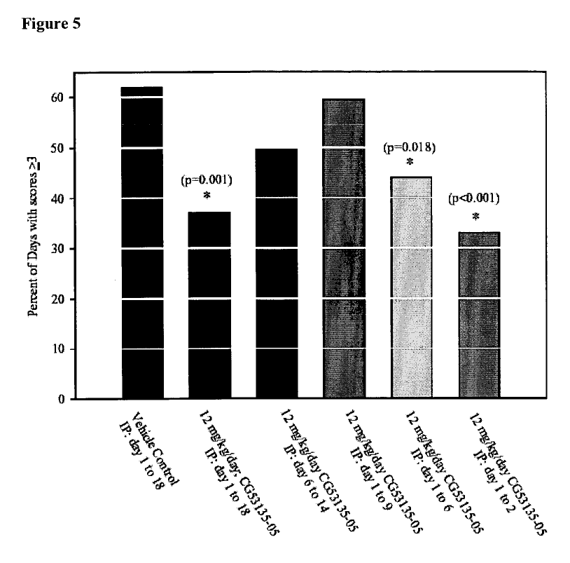

In this model of mucositis primarily induced by chemotherapy, dosing schedule was important in the treatment of oral mucositis. Administration of CG53135-05 (12 mg/kg/day) from Day 6 to Day 14 or Day 1 to Day 9 did not result in significant improvement in the course or severity of mucositis (FIG. 5). Administration of CG53135-05 (12 mg/kg/day) from Day 1 to Day 18 or Day 1 to Day 6 resulted in significant improvement of the duration of severe mucositis (Chi-square analysis); however these treatments did not result in significant improvement of daily mucositis scores (rank sum analysis) (data not shown). Treatment with 12 mg/kg/day CG53135-05 (Day 1 to Day 2) had a significant effect on both the course and severity of mucositis in this study (FIG. 5). These results suggest that a short-course of treatment with CG53135-05 immediately after a combined chemotherapy and radiation regimen improves the outcome of the disease in this model of mucositis.

In another experiment, treatment of hamsters with 12 mg/kg/day CG53135-05 starting after radiation (Day 1 to Day 18) resulted in a significant reduction of ulceration (p <0.001) combined with 7 days of significant reduction in mucositis scores, as determined by rank sum analysis (N-198, data not shown). This suggests that the administration of CG53135-05 results in a significantly beneficial treatment of radiation-induced oral mucositis when administered after mucosatoxic insult.

In yet another experiment, administration of 12 mg/kg/day of CG53135-05 (formulated in 40 mM sodium acetate, 0.2 M L-arginine, and 3% glycerol) on Days 1 to 2 significantly reduced the severity of mucositis (N-237, data not shown). These results confirm the findings presented above.

Conclusions

The activity of CG53135 was evaluated in a model of mucositis induced in hamsters treated with 60 mg/kg 5-flourouracil on Days -4 and -2, followed by a single non-mucosatoxic dose of radiation (˜30 Gy) on Day 0. Clinically relevant oral mucositis (i.e., mucositis score of ≧3) developed Day 15. Prophylactic administration of CG53135 (i.e., prior to or concurrent with the insult) had no beneficial effect and could worsen mucositis. Intraperitoneal administration of CG53135 for 2, 6, or 18 days significantly reduced severity of mucositis. No studies were conducted using an intravenous (IV) route of administration since IV administration in hamsters is technically challenging and data are consequently highly variable.

### Example 7

Effect of CG53135-05 Administration on Hamster Epithelial Proliferation In Vivo (N-225)

### BACKGROUND

The inventors have demonstrated the utility of CG53135-05 in the reduction of severity of oral mucositis in the hamster model. Furthermore, the experiment described herein, was to evaluate in vivo incorporation of BrdU into the gastrointestinal epithelium and bone marrow after a single dose of CG53135-05.

Materials and Methods

Study Design Male Golden Syrian hamsters (Charles River Laboratories or Harlan Sprague Dawley), aged 5 to 6 weeks, with a mean body weight of 82 g at study commencement, were used. Twenty-five male hamsters were randomized into 5 groups of 5 animals each as outlined in Table 9.

TABLE 9
Treatment Groups

| Group | No. of | | Euthanasia/ | Volume (mL); |
|---|---|---|---|---|
| No. | Animals | Treatment | Necropsy | Treatment |
| 1 | 5 males | BrdU 50 mg/kg, IP, (0 hrs) | 2 hrs | Adjust by body weight |
| 2 | 5 males | 12 mg/kg CG53135-05, IP (0 hrs) + BrdU | 2 hrs | Adjust by body weight |
| | | 50 mg/kg, IP, (0 hrs) | | |
| 3 | 5 males | 12 mg/kg CG53135-05, IP (0 hrs) + BrdU | 4 hrs | Adjust by body weight |
| | | 50 mg/kg, IP, (2 hrs) | | |
| 4 | 5 males | 12 mg/kg CG53135-05, IP (0 hrs) + BrdU | 8 hrs | Adjust by body weight |
| | | 50 mg/kg, IP, (6 hrs) | | |
| 5 | 5 males | 12 mg/kg CG53135-05, IP (0 hrs) + BrdU | 24 hrs | Adjust by body weight |
| | | 50 mg/kg, IP, (22 hrs) | | |

A single dose of CG53135-05 at 12 mg/kg IP was administered and hamsters were sacrificed at 2, 4, 8 and 24 hours post-administration.

BrdU Administration and Immunohistochemistry

All animals received BrdU 50 mg/kg IP two hours before sacrifice, allowing for uptake of the reagent into proliferating tissues. At euthanasia, the following tissues were harvested: cheek pouch mucosa, esophagus, stomach, duodenum, jejunum, ileum, cecum, colon, rectum and sternum. All tissue samples were fixed in 10% neutral buffered formalin for 24 hrs and then transferred to 70% ethanol. Samples were trimmed, paraffin embedded, sectioned and mounted. Epithelial tissues were stained for incorporation of BrdU by immunohistochemistry using Oncogene Research products BrdU Immunohistochemistry kit Catalog # HCS24 in accordance with the manufacturer's instructions.

Results

The effect of CG53135-05 on the incorporation of BrdU into all tissues was essentially the same: a relatively small increase in the number of BrdU labeled nuclei was observed 2 hours after the administration of CG53 135-05 (data not shown). This was followed by a decrease in the number of labeled nuclei at 4 hours after the administration of CG53135-05. All tissues showed a dramatic increase in BrdU labeling at 8 hours post administration. At 24 hours, all tissues except rectum showed a decrease in the number of labeled nuclei compared with the untreated controls, while the rectal tissue showed a slight increase over the controls. Since no labeled cells were seen in the rectal tissue samples from the untreated animals, the observation of 2 labeled cells in the 24 hour time point has to be regarded as observational error, or data scatter, since there must be a low level of cell replication in the tissue.

Conclusions

The in vivo mechanistic activity of CG53135 was evaluated using bromodexoyuridine labeling in vivo to evaluate the effect of a single bolus dose (12 mg/kg) of CG53135-05 on mucosal tissue over a 24-hour period. CG53135-05 stimulated the division of the epithelial cells of the cheek pouch, jejunum and rectum as well as the hemopoetic cells of the bone marrow. Peak increases in BrdU incorporation in these tissues were seen at 8 hours after the administration of CG53135-05. All tissues showed the same time response to the administration of CG53135-05.

### Example 8

CG53135-05: Drug Product Formulation and Composition

Materials and Methods

Several constructs were made to produce protein for nonclinical studies: tagged full-length (CG53135-01), untagged codon-optimized deletion-mutant (CG53135-02), and untagged codon-optimized full-length (CG53135-05), all of which are described in Section I, Example 3. Aiming for a construct that would be suitable for clinical development, untagged molecules were generated in a phage-free bacterial host. The codon-optimized, full-length, untagged molecule (CG53135-05) has the most favorable pharmacology profile and was used to prepare product for the safety studies and clinical trial.

CG53135-05 was expressed in Escherichia coli BLR (DE3) using a codon-optimized construct, purified to homogeneity, and characterized by standard protein chemistry techniques. The isolated CG53135-05 protein migrated as a single band (23 kilodalton) using standard SDS-PAGE techniques and stained with Coommassie blue (data not shown). The CG53135-05 protein was electrophoretically transferred to a polyvinylidenefluoride membrane and the stained 23 kD band was excised from the membrane and analyzed by an automated Edman sequencer (Procise, Applied Biosystems, Foster City, Calif.); the N-terminal amino acid sequence of the first 10 amino acids was confirmed as identical to the predicted protein sequence (data not shown).

Fermentation and Primary Recovery Recombinant CG53135-05 was expressed using Escherichia coli (E. coli) BLR (DE3) cells (Novagen). These cells were transformed with full length, codon optimized CG53135-05 using pET24a vector (Novagen). A Manufacturing Master Cell Bank (MMCB) of these cells was produced and qualified. The fermentation and primary recovery processes were performed at the 100 L (i.e., working volume) scale reproducibly.

Seed preparation was started by thawing and pooling of 1-6 vials of the MMCB and inoculating 4-7 shake flasks each containing 750 mL of seed medium. At this point, 3-6 L of inoculum was transferred to a production fermentor containing 60-80 L of start-up medium. The production fermentor was operated at a temperature of 37° C. and pH of 7.1. Dissolved oxygen was controlled at 30% of saturation concentration or above by manipulating agitation speed, air sparging rate and enrichment of air with pure oxygen. Addition of feed medium was initiated at a cell density of 30-40 AU (600 nm) and maintained until end of fermentation. The cells were induced at a cell density of 40-50 AU (600 nm) using 1 mM isopropyl-beta-D-thiogalactoside (IPTG) and CG53135-05 protein was produced for 4 hours post-induction. The fermentation was completed in 10-14 hours and about 100˜110 L of cell broth was concentrated using a continuous centrifuge. The resulting cell paste was stored frozen at −70° C.

The frozen cell paste was suspended in lysis buffer (containing 3M urea, final concentration) and disrupted by high-pressure homogenization. The cell lysate was clarified using continuous flow centrifugation. The resulting clarified lysate was directly loaded onto a SP-sepharose Fast Flow column equilibrated with SP equilibration buffer (3 M urea, 100 mM sodium phosphate, 20 mM sodium chloride, 5 mM EDTA, pH 7.4). CG53135-05 protein was eluted from the column using SP elution buffer (100 mM sodium citrate, 1 M arginine, 5 mM EDTA, pH 6.0). The collected material was then diluted with an equal volume of SP elution buffer. After thorough mixing, the SP Sepharose FF pool was filtered through a 0.2 μm PES filter and frozen at −80° C.

Purification of the Drug Substance: The SP-sepharose Fast Flow pool was precipitated with ammonium sulfate. After overnight incubation at 4° C., the precipitate was collected by bottle centrifugation and subsequently solubilized in Phenyl loading buffer (100 mM sodium citrate, 500 mM L-arginine, 750 mM NaCl, 5 mM EDTA, pH 6.0). The resulting solution was filtered through a 0.45 uM PES filter and loaded onto a Phenyl-sepharose HP column. After washing the column, the protein was eluted with a linear gradient with Phenyl elution buffer (100 mM sodium citrate, 500 mM L-arginine, 5 mM EDTA, pH 6.0). The Phenyl-sepharose HP pool was filtered through a 0.2 μm PES filter and frozen at −80° C. in 1.8 L aliquots.

Formulation and Fill/Finish Four batches of purified drug substance were thawed for 24-48 h at 2-8° C. and pooled into the collection tank of tangential flow ultrafiltration (TFF) equipment. The pooled drug substance was concentrated ˜5-fold via TFF, followed by ˜5-fold diafiltration with the formulation buffer (40 mM sodium acetate, 0.2 M L-arginine, 3% glycerol). This buffer-exchanged drug substance was concentrated further to a target concentration of >10 mg/mL. Upon transfer to a collection tank, the concentration was adjusted to ˜10 mg/mL with formulation buffer. The formulated drug product was sterile-filtered into a sterile tank and aseptically filled (at 10.5 mL per 20 mL vial) and sealed. The filled and sealed vials were inspected for fill accuracy and visual defects. A specified number of vials were drawn and labeled for release assays, stability studies, safety studies, and retained samples. The remaining vials were labeled for the clinical study, and finished drug product was stored at −80±15° C.

Results

The finished drug product is a sterile, clear, colorless solution in single-use sterile vials for injection. CG53135-05 was formulated at a final concentration of 8.2 mg/mL (Table 10).

TABLE 10
Composition of Drug Product

| | | Final | | Amount | |
|---|---|---|---|---|---|
| Component | Grade | Concentration | | per Liter | |
| CG53135-05 | NA | 8.2 | mg/mL | 8.2 | g |

Formulation Buffer

| | | | | | |
|---|---|---|---|---|---|
| Sodium acetate (trihydrate) | USP | 40 | mM | 5.44 | g |
| L-arginine HCl | USP | 200 | mM | 42.132 | g |

| | | | | |
|---|---|---|---|---|
| Glycerol | USP | 3% v/v | 30 | mL |

| | | | |
|---|---|---|---|
| Acetic acid | USP | NA | QS to pH 5.3 |
| Water for injection | USP | NA | QS to 1 L |

NA = not applicable;
QS = quantity sufficient

The pharmacokinetics of optimally-formulated CG53135-05 was assessed in rats following intravenous, subcutaneous, and intraperitoneal administration to compare exposure at active doses in animal models and predict exposure in humans (Study N-128; data not shown). Intravenous administration of CG53135-05 resulted in high plasma levels (maximum plasma level=19, 680-47,252 ng/mL), which rapidly declined within the first 2 h to 30-70 ng/mL; decreased exposure was observed following the third daily dose (maximum plasma level=5373-7453 ng/mL). Subcutaneous administration of CG53135-05 resulted in slow absorption (maximum plasma level at 10 h) and plasma levels of 40-80 ng/mL up to 48 h after dosing; some accumulation in plasma was seen following the third daily dose. Intraperitoneal administration of CG53135-05 resulted in slow absorption (maximum plasma level at 2-4 h) and plasma levels of 40-70 ng/mL up to 10 h after dosing; decreased exposure was seen following third daily dose. No significant gender differences were observed by any route of administration.

Safety of intravenous administration of CG53135 (0.05, 5 or 50 mg/kg/day for 14 consecutive days) was assessed in a pivotal toxicology study in rats (Study N-127; data not shown). There were no treatment-related findings in rats administered 0.05 mg/mL CG53135 for 14 days. In rats administered 5 mg/kg CG53135 for 14 days, food consumption was reduced and body weight was decreased; while there were no treatment-related changes in organ weights, urinalysis, ophthalmology, or histopathology parameters in this dose group, there were treatment-related changes in hematology and clinical chemistry parameters in this treatment group. In rats administered 50 mg/kg CG53135 for 12 days (estimated maximum plasma level of 20-30 fold higher than active dose), food consumption was reduced and body weight was markedly decreased; while there were no treatment-related changes in ophthalmology, there were significant treatment-related changes in organ weights, urinalysis, hematology, clinical chemistry, and histopathology in this treatment group.

Safety of intravenous administration of CG53135 (0 or 10 mg/kg/day for 7 consecutive days) was further assessed in a safety pharmacology study in rhesus monkeys. There were no treatment-related clinical observations in animals administered 1 mg/kg CG53135 for 7 days (Study N-235; data not shown). In animals administered 10 mg/kg CG53135 for 7 days, minor effects on body weight were noted and associated with qualitative observations of lower food consumption. There were no apparent treatment-related effects on hematology, clinical chemistry, ophthalmology, or electrophysiology in either dose group.

### Example 9

Stability of CG53135-05 Drug Substance

Materials and Methods

The inventors have performed stability studies on the purified CG53135-05 drug substance produced during cGMP manufacturing. The analytical methods used as stability indicating assays for purified drug substance are listed in Table 11.

TABLE 11
Stability Assays for Drug Substance

| Assay | Stability Criteria |
|---|---|
| SDS-PAGE (Neuhoff stain) | >98% pure by densitometry |
| | (reduced and nonreduced) |
| RP-HPLC | Peak at 5.5 ± 1.0 min |
| | relative retention time |
| SEC-HPLC | >90% mono-disperse peak |
| Total protein by Bradford method | >0.2 mg/mL |
| Bioassay (BrdU) | PI₂₀₀ > 0.5 ng/mL and <20 ng/mL |
| pH | 5.8 ± 0.4 |
| Visual appearance | Clear and colorless |

PI₂₀₀ = concentration of CG53135-05 that results in incorporation of BrdU at 2 times the background

The SDS-PAGE, RP-HPLC, and Bradford assays are indicative of protein degradation or gross aggregation. The SEC-HPLC assay detects aggregation of the protein or changes in oligomerization, and the bioassay detects loss of biological activity of the protein. The stability studies for the purified drug substance were conducted at −80 to 15° C. with samples tested at intervals of 3, 6, 9, 12, and 24 months.

Results and Conclusions

In one experiment, stability studies of finished drug product were conducted by Cambrex at −80±15° C. and −20±5° C. with samples tested at intervals of 1, 3, 6, 9, 12, and 24 months. Stability data collected after 1 month indicate that finished drug product is stable for at least 1 month when stored at −80±15° C. or at −20±5° C. (Table 12).

TABLE 12
Stability Data for Drug Product after 1-month interval

| Assay | Stability Criteria | Initial | −80 ± 15° C. | −20 ± 5° C. |
|---|---|---|---|---|
| RP-HPLC | Major peak retention | Major peak | Major peak | Major peak |
| | time ± 0.2 min | retention time ± | retention time ± | retention time ± |
| | relative to Reference | 0.2 min relative to | 0.2 min relative to | 0.2 min relative to |
| | Standard | Reference | Reference | Reference |
| | | Standard | Standard | Standard |
| SDS-PAGE | Major band migrates | Pass | Pass | Pass |
| | at about 23 kDa; | | | |
| | nonreduced minor | | | |
| | band below major | | | |
| | band | | | |
| SEC-HPLC | >90% mono- | 100% | 100% | 100% |
| | disperse peak | | | |
| Bradford | 10 ± 0.2 mg/mL | 8.2 | 8.6 | 8.3 |
| Bioassay | PI₂₀₀ > 0.5 ng/mL and | 4.14 ng/mL | 2.98 ng/mL | 1/45 ng/mL |
| | <20 ng/mL | | | |
| Sterility | Pass (ie., no growth) | Pass | NT | NT |
| pH | 5.3 ± 0.3 | 5.4 | 5.5 | 5.4 |
| Visual | Clear and colorless | Pass | Pass | Pass |
| appearance | solution | | | |

Lot # 02502001 was stored at −80 ± 15° C. or at −20 ± 5° C. at Cambrex and tested after 1 month;
PI₂₀₀ = concentration of CG53135-05 that results in incorporation of BrdU at 2 times the background;
Pass = results met stability criterion;
NT = not tested

In another experiment, samples of finished drug product were stored at −80±15° C. or stressed at 5±3° C., 25±2° C., or 37±2° C. and tested at various intervals for 1 month (data not shown). Stability data indicate that finished drug product showed no significant instability after 1 month of storage at −80±15° C. or 5±3° C. When stressed at 25±2° C., finished drug product was stable for at least 48 hours; degradation was apparent after 1 week at this temperature. When stressed at 37±2° C., degradation of finished drug product was apparent within 4 hours.

### Example 10

Prophetic Dosing Schedule for Human Phase I Clinical Trials (C-214) for Oral Mucositis

Dose selection for a single, rising-dose first-in-human trial in patients with colorectal cancer (Study C-214), which includes doses of 0, 0.1, 0.3, 1, or 3 mg/kg CG53135, is based primarily on pharmacology and toxicology data summarized in Section II. The active dose of CG53135-05 in models of mucositis (Examples 7 and 8) was 12 mg/kg/day with maximum plasma levels of ˜600 ng/mL. The inventors modeled from existing rat pharmacokinetics data, making assumptions of dose-linear pharmacokinetics following intravenous administration and decreased clearance rates in humans (compared to exposure when administered to rodents). Further modeling for a 15 minute intravenous infusion established that an infusion of 1 mg/kg/day would achieve maximum plasma levels of ˜800 ng/mL, comparable to the maximum plasma levels in hamsters upon intraperitoneal administration of 12 mg/kg. Thus, based on the in vivo studies, it is expected that the active dose of CG53135 in humans to be 1 mg/kg/day. At the lowest dose for Study C-214 (i.e., 0.1 mg/kg), the maximum plasma level in humans (estimated to be 80 ng/mL) is expected to be lower than the calculated maximum plasma level at the no-effect dose in rats (i.e., maximum plasma level at 0.05 mg/kg/day CG53135 estimated at 100 ng/mL).

CG53135 can be safely administered to humans at the selected doses for Study C-214 (i.e., 0, 0.1, 0.3, 1, or 3 mg/kg administered as a single intravenous infusion to inpatients with Stage 4 colorectal cancer). The starting dose is about 1/10th of the no adverse effect level in rats and in non-human primates (defined after 7-14 consecutive days of intravenous administration of CG53135). In addition, adverse events that can be anticipated based on animal toxicology studies are reversible and can be readily monitored in enrolled patients.

What is claimed is:

1. A method of treating, preventing, or delaying a tissue proliferation-associated disorder comprising administering to a subject a therapeutically effective amount of an isolated polypeptide selected from the group consisting of:
a) a polypeptide comprising the amino acid sequence selected from the group consisting of SEQ ID NOS: 2, 4, 6, 8, 10, 12, 14, 16, 18, 20, 22, 24, 26, 28, 30, 32, 34, and 36;
b) a mature form of a polypeptide comprising the amino acid sequence selected from the group consisting of SEQ ID NOS: 2, 4, 6, 8, 10, 12, 14, 16, 18, 20, 22, 24, 26, 28, 30, 32, 34, and 36;
c) the polypeptide of (a) and (b), wherein one or more amino acid substitutions are made to the polypeptide to produce a variant, provided that the variant is no more than 15% divergent in sequence from the polypeptide, and provided that said variant retains cellular proliferation activity;
d) a fragment of the polypeptide of (a), (b), or (c), which fragment retains cellular proliferation activity.

2. The method of claim 1, wherein the subject is a mammal.

3. The method of claim 2, wherein the mammal is a human.

4. The method of claim 1, wherein the tissue proliferation-associated disorder is oral mucositis.

5. The method of claim 1, wherein the tissue proliferation-associated disorder occurs in the mouth.

6. The method of claim 1, wherein the tissue proliferation-associated disorder is oral candidiasis.

7. The method of claim 1, wherein administering comprises providing said polypeptide to the subject intravenously.

8. The method of claim 1, wherein administering comprises providing said polypeptide to the subject subcutaneously.

9. The method of claim 1, wherein administering comprises providing-said polypeptide to the subject in a mouthwash solution or topical ointment.

10. The method of claim 1, wherein said therapeuticaly effective amount is from about 0 mg/kg/day to about 3 mg/kg/day.

11. The method of claim 10, wherein said therapeutically effective amount is about 1 mg/kg/day.

12. A method of preparing a pharmaceutical composition comprising combining at least one polypeptide effective in treating, preventing, or delaying a tissue proliferation-associated disorder with a pharmaceutically acceptable carrier, wherein the polypeptide is selected from the group consisting of:.
a) a polypeptide comprising the amino acid sequence selected from the group consisting of SEQ ID NOS: 2, 4, 6, 8, 10, 12, 14, 16, 18, 20, 22, 24, 26, 28, 30, 32, 34, and 36;
b) a mature form of a polypeptide comprising the amino acid sequence selected from the group consisting of SEQ ID NOS: 2, 4, 6, 8, 10, 12, 14, 16, 18, 20, 22, 24, 26, 28, 30, 32, 34, and 36;
c) the polypeptide of (a) and (b), wherein one or more amino acid substitutions are made to the polypeptide to produce a variant, provided that the variant is no more than 15% divergent in sequence from the polypeptide, and provided that said variant retains cellular proliferation activity;
d) a fragment of the polypeptide of (a), (b), or (c), which fragment retains cellular proliferation activity.

13. The method of claim 12, wherein the tissue proliferation-associated disorder is oral mucositis, a tissue proliferation associated-disorder that occurs in the mouth, or oral candidiasis.

14. The method of claim 12, wherein the pharmaceutical composition is suitable for intravenous, subcutaneous, or transmucosal administration to a subject.

15. The method of claim 14, wherein the subject is a mammal.

16. The method of claim 15, wherein the mammal is a human.

17. A method for determining the presence of or predisposition to a tissue proliferation-associated disorder associated with altered levels of a nucleic acid molecule encoding the polypeptide decribed in claim 1 in a first mammalian subject, the method comprising:
a) measuring the amount of the nucleic acid in a sample from the first mammalian subject; and
b) comparing the amount of the nucleic acid in the sample of step (a) to the amount of the nucleic acid present in a control sample from a second mammalian subject known not to have, or not to be predisposed to, the disorder;
wherein an alteration in the level of the nucleic acid in the first subject as compared to the control sample indicates the presence of or predisposition to the disorder.

18. An aqueous drug formulation for treating, preventing, or delaying a tissue proliferation-associated disorder in a subject comprising:
a) a therapeutically effective amount of an isolated polypeptide selected from the group consisting of:
i) a polypeptide comprising the amino acid sequence selected from the group consisting of SEQ ID NOS: 2, 4, 6, 8, 10, 12, 14, 16, 18, 20, 22, 24, 26, 28, 30, 32, 34, and 36;
ii) a mature form of a polypeptide comprising the amino acid sequence selected from the group consisting of SEQ ID NOS: 2, 4, 6, 8, 10, 12, 14, 16, 18, 20, 22, 24, 26, 28, 30, 32, 34, and 36;
iii) the polypeptide of (i) and (ii), wherein one or more amino acid substitutions are made to the polypeptide to produce a variant, provided that the variant is no more than 15% divergent in sequence from the polypeptide, and provided that said variant retains cellular proliferation activity;
iv) a fragment of the polypeptide of (i), (ii), or (iii), which fragment retains cellular proliferation activity; and
b) a formulation buffer.

19. The drug formulation of claim 18, wherein the formulation buffer comprises 40 mM sodium acetate, 200 mM L-Arginine, and 3% by volume glycerol, per liter of aqueous material suitable for injection.

20. The drug formulation buffer of claim 18, wherein the pH is from about 4.9 to about 6.2.

21. The drug formulation of claim 18, wherein the pH is about 5.3.

22. The drug formulation of claim 18, wherein said formulation is stable for at least one month at about −95° C. to about 8° C.

23. A method of promoting the proliferation of a mammalian cell comprising contacting the cell with a polypeptide comprising the amino acid sequence selected from the group consisting of:
i) a polypeptide comprising the amino acid sequence selected from the group consisting of SEQ ID NOS: 2, 4, 6, 8, 10, 12, 14, 16, 18, 20, 22, 24, 26, 28, 30, 32, 34, and 36;
ii) a mature form of a polypeptide comprising the amino acid sequence selected from the group consisting of SEQ ID NOS: 2, 4, 6, 8, 10, 12, 14, 16, 18, 20, 22, 24, 26, 28, 30, 32, 34, and 36;
iii) the polypeptide of (i) and (ii), wherein one or more amino acid substitutions are made to the polypeptide to produce a variant, provided that the variant is no more than 15% divergent in sequence from the polypeptide, and provided that said variant retains cellular proliferation activity;
iv) a fragment of the polypeptide of (i), (ii), or (iii), which fragment retains cellular proliferation activity, wherein the polypeptide or fragment has at least one property selected from the group consisting of:
a) inducing proliferation of mammmalian cells; and
b) inducing growth of mammalian cell

24. The method of claim 23, wherein the mammalian cell is of mesenchymal, epithelial, or endothelial origin.

25. The method of claim 1, wherein the polypeptide of (a) further comprises a post-translational modification.

26. The method of claim 25, wherein the post-translational modification is at least one modification chosen from the group consisting of phosphorylation, glycosolation, and N-myristoylation.


SEQUENCE LISTING (36 sequences)

SEQ ID NO: 1 - DNA, 636 bp, Homo sapiens
  CDS (1)..(633)
atggctccgctggctgaagttggtggtttcctgggcggtctggagggtctgggtcagcaggttggttctcacttcctgctgccgccggctggtgaacgtccgccactgctgggtgaacgtcgctccgcagctgaacgctccgctcgtggtggcccgggtgctgctcagctggctcacctgcatggtatcctgcgtcgccgtcagctgtactgccgtactggtttccacctgcagatcctgccggatggttctgttcagggtacccgtcaggaccactctctgttcggtatcctggaattcatctctgttgctgttggtctggtttctatccgtggtgttgactctggcctgtacctgggtatgaacgacaaaggcgaactgtacggttctgaaaaactgacctctgaatgcatcttccgtgaacagtttgaagagaactggtacaacacctactcttccaacatctacaaacatggtgacaccggccgtcgctacttcgttgctctgaacaaagacggtaccccgcgtgatggtgctcgttctaaacgtcaccagaaattcacccacttcctgccgcgcccagttgacccggagcgtgttccagaactgtataaagacctgctgatgtacacctaa

SEQ ID NO: 2 - protein, 211 aa, Homo sapiens
MAPLAEVGGFLGGLEGLGQQVGSHFLLPPAGERPPLLGERRSAAERSARGGPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 3 - DNA, 633 bp, Homo sapiens
  CDS (1)..(633)
atggctcccttagccgaagtcgggggctttctgggcggcctggagggcttgggccagcaggtgggttcgcatttcctgttgcctcctgccggggagcggccgccgctgctgggcgagcgcaggagcgcggcggagcggagcgcgcgcggcgggccgggggctgcgcagctggcgcacctgcacggcatcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaaggacctactgatgtacact

SEQ ID NO: 4 - protein, 211 aa, Homo sapiens; identical to SEQ ID NO: 2

SEQ ID NO: 5 - DNA, 540 bp, Homo sapiens
  CDS (1)..(537)
atggctcccttagccgaagtcgggggctttctgggcggcctggagggcttgggccagccgggggcagcgcagctggcgcacctgcacggcatcctgcgccgccggcagctctattgccgcaccggcttccacctgcagatcctgcccgacggcagcgcgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaaggacctactgatgtacacttag

SEQ ID NO: 6 - protein, 179 aa, Homo sapiens
MAPLAEVGGFLGGLEGLGQPGAAQLAHLHGILRRRQLYCRTGFHLQILPDGSAQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 7 - DNA, 556 bp, Homo sapiens
  CDS (2)..(556)
caccagatctatggctcccttagccgaagtcgggggctttctgggcggcctggagggcttgggccagccgggggcagcgcagctggcgcacctgcacggcatcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaaggacctactgatgtacactgtcgacggc

SEQ ID NO: 8 - protein, 185 aa, Homo sapiens
TRSMAPLAEVGGFLGGLEGLGQPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYTVDG

SEQ ID NO: 9 - DNA, 415 bp, Homo sapiens
  CDS (2)..(415)
caccagatctatcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtcgacggc

SEQ ID NO: 10 - protein, 138 aa, Homo sapiens
TRSILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDG

SEQ ID NO: 11 - DNA, 466 bp, Homo sapiens
  CDS (2)..(466)
caccagatctatcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaaggacctactgatgtacactgtcgacggc

SEQ ID NO: 12 - protein, 155 aa, Homo sapiens
TRSILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYTVDG

SEQ ID NO: 13 - DNA, 549 bp, Homo sapiens
  CDS (1)..(549)
agatctatggctcccttagccgaagtcgggggctttctgggcggcctggagggcttgggccagccgggggcagcgcagctggcgcacctgcacggcatcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaaggacctactgatgtacactctcgag

SEQ ID NO: 14 - protein, 183 aa, Homo sapiens
RSMAPLAEVGGFLGGLEGLGQPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYTLE

SEQ ID NO: 15 - DNA, 408 bp, Homo sapiens
  CDS (1)..(408)
agatctatcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatgaagacactggccgcaggtattttgtggcacttaacaaagacggaactccaagagatggcgccaggtccaagaggcatcagaaatttacacatttcttacctagaccactcgag

SEQ ID NO: 16 - protein, 136 aa, Homo sapiens
RSILRRRQLYCRTGFHLQILPDGSVQGTRQDHSLFGILEFISVAVGLVSIRGVDSGLYLGMNDKGELYGSEKLTSECIFREQFEENWYNTYSSNIYKHEDTGRRYFVALNKDGTPRDGARSKRHQKFTHFLPRPLE

SEQ ID NO: 17 - DNA, 408 bp, Homo sapiens
  CDS (1)..(408)
agatctatcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccactcgag

SEQ ID NO: 18 - protein, 136 aa, Homo sapiens
RSILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPLE

SEQ ID NO: 19 - DNA, 477 bp, Homo sapiens
  CDS (1)..(474)
atggctcagctggctcacctgcatggtatcctgcgtcgccgtcagctgtactgccgtactggtttccacctgcagatcctgccggatggttctgttcagggtacccgtcaggaccactctctgttcggtatcctggaattcatctctgttgctgttggtctggtttctatccgtggtgttgactctggcctgtacctgggtatgaacgacaaaggcgaactgtacggttctgaaaaactgacctctgaatgcatcttccgtgaacagtttgaagagaactggtacaacacctactcttccaacatctacaaacatggtgacaccggccgtcgctacttcgttgctctgaacaaagacggtaccccgcgtgatggtgctcgttctaaacgtcaccagaaattcacccacttcctgccgcgcccagttgacccggagcgtgttccagaactgtataaagacctgctgatgtacacctaa

SEQ ID NO: 20 - protein, 158 aa, Homo sapiens
MAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 21 - DNA, 636 bp, Homo sapiens
  CDS (1)..(633)
atggctcccttagccgaagtcgggggctttctgggcggcctggagggcttgggccagcaggtgggttcgcatttcctgttgcctcctgccggggagcggccgccgctgctgggcgagcgcaggagcgcggcggagcggagcgcgcgcggcgggccgggggctgcgcagctggcgcacctgcacggcatcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaaggacctactgatgtacacttga

SEQ ID NO: 22 - protein, 211 aa, Homo sapiens; identical to SEQ ID NO: 2

SEQ ID NO: 23 - DNA, 540 bp, Homo sapiens
  CDS (1)..(537)
atggctcccttagccgaagtcgggggctttctgggcggcctggagggcttgggccagccgggggcagcgcagctggcgcacctgcacggcatcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaaggacctactgatgtacacttag

SEQ ID NO: 24 - protein, 179 aa, Homo sapiens
MAPLAEVGGFLGGLEGLGQPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 25 - DNA, 54 bp, Homo sapiens
  CDS (1)..(54)
atggctcccttagccgaagtcgggggctttctgggcggcctggagggcttgggc

SEQ ID NO: 26 - protein, 18 aa, Homo sapiens
MAPLAEVGGFLGGLEGLG

SEQ ID NO: 27 - DNA, 63 bp, Homo sapiens
  CDS (1)..(63)
gagcggccgccgctgctgggcgagcgcaggagcgcggcggagcggagcgcgcgcggcgggccg

SEQ ID NO: 28 - protein, 21 aa, Homo sapiens
ERPPLLGERRSAAERSARGGP

SEQ ID NO: 29 - DNA, 63 bp, Homo sapiens
  CDS (1)..(63)
cgcaggtattttgtggcacttaacaaagacggaactccaagagatggcgccaggtccaagagg

SEQ ID NO: 30 - protein, 21 aa, Homo sapiens
RRYFVALNKDGTPRDGARSKR

SEQ ID NO: 31 - DNA, 60 bp, Homo sapiens
  CDS (1)..(60)
cctagaccagtggatccagaaagagttccagaattgtacaaggacctactgatgtacact

SEQ ID NO: 32 - protein, 20 aa, Homo sapiens
PRPVDPERVPELYKDLLMYT

SEQ ID NO: 33 - DNA, 51 bp, Homo sapiens
  CDS (1)..(51)
atgaacgacaagggcgagctgtacggcagcgagaagctgaccagcgagtgc

SEQ ID NO: 34 - protein, 17 aa, Homo sapiens
MNDKGELYGSEKLTSEC

SEQ ID NO: 35 - DNA, 633 bp, Homo sapiens
  CDS (1)..(633)
atggctcccttagccgaagtcgggggctttctgggcggcctggagggcttgggccagcaggtgggttcgcatttcctgttgcctcctgccggggagcggccgccgctgctgggcgagcgcaggagcgcggcggagcggagcgcgcgcggcgggccgggggctgcgcagctggcgcacctgcacggcatcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaagaacctactgatgtacact

SEQ ID NO: 36 - protein, 211 aa, Homo sapiens [sequence omitted]